(19) United States
(12) Patent Application Publication
(10) Pub. No.: US 2026/0217667 A1
(43) Pub. Date: Jul. 30, 2026

(54) AMYLOID TARGETING AGENTS

(71) Applicant: Amydis, Inc., Los Angeles, CA (US)

(72) Inventors: Stella Sarraf, Beverly Hills, CA (US); Gerald Swiss, San Diego, CA (US)

(21) Appl. No.: 19/146,892

(22) PCT Filed: Jan. 17, 2024
(86) PCT No.: PCT/US2024/011789

§ 371 Date: Jul. 9, 2025

Related U.S. Application Data
(60) Provisional application No. 63/480,453, filed on Jan. 18, 2023.

(51) Int. Cl. C07D 295/155; A61K 31/445; A61K 31/496; A61K 31/5375; A61K 49/00; A61P 25/28; C07D 211/18; C07D 241/04; C07D 265/30

(52) U.S. Cl. CPC C07D 295/155; A61K 31/445; A61K 31/496; A61K 31/5375; A61K 49/0021; A61P 25/28; C07D 211/18; C07D 241/04; C07D 265/30

(57) ABSTRACT

Provided herein is the design and synthesis of novel molecular rotor fluorophores useful for detection of amyloid or amyloid like proteins. The fluorophores are designed to exhibit enhanced fluorescence emission upon associating with amyloid or amyloid like proteins as compared to unbound compound. Also disclosed herein are the methods for treating of diseases associated with an amyloid or amyloid like proteins.

### CROSS-REFERENCE TO RELATED APPLICATIONS

This application claims the benefit under 35 U.S.C. § 119(e) to U.S. Provisional Application No. 63/480,453, filed Jan. 18, 2023, all of which is hereby incorporated by reference in its entirety.

### BACKGROUND

Amyloid plaque accumulation in the brain is the hallmark of many neurodegenerative disorders, including Alzheimer's disease (AD), Parkinson's disease, Down syndrome, and Creutzfeldt-Jakob disease (CJD). There are now a number of approaches to both clinically diagnose and monitor the progression of these diseases which include targeting of amyloid deposits with small-molecule imaging agents. One such approach involves fluorescence-based small molecule imaging of amyloids which is a low cost, accessible, and non-radioactive technique for the detection of the amyloid deposits.

Ideally, the fluorescent-based small molecules should have enhanced emission brightness, spectroscopic properties that identify amyloid plaque binding, and specificity for binding amyloids in neuronal tissue in order to provide meaningful diagnostic data. In addition, such small molecules should exhibit superior chemical/hydrolytic stability in physiologically relevant solutions over the time required to assess the presence and extent of amyloid plaque accumulation obtained in such labeling assays.

### SUMMARY

Disclosed are compounds that are fluorescent-based small molecules that possess such criteria as described above. When used in diagnostic assays, these compounds can accurately assess the presence and extent of amyloid plaque accumulation. Specifically, disclosed is a compound of formula I or II:

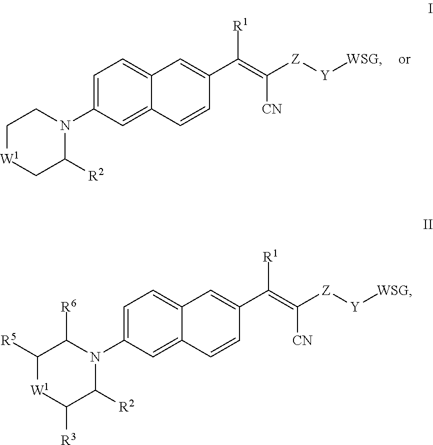

or a pharmaceutically acceptable salt, tautomer or a prodrug thereof wherein:
W¹ is O, N(R¹⁴), or C(R⁴)₂;
R¹⁴ is hydrogen, alkyl, halomethyl having 1 to 3 halo groups, —COOH, —CONH₂, substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is selected from hydrogen, halo, haloalkyl having 1 to 3 halo groups, or cyano;
R², R³, R⁴, R⁵, and R⁶ are each independently hydrogen, halo, haloalkyl having 1 to 3 halo groups, —CN, —C(O)R^{1A}, —C(O)OR^{1A}, —C(O)NR^{1A}R^{1B}, —OR^{1A}, —NHC(O)NR^{1A}R^{1B}, —NR^{1A}R^{1B}, —NHNR^{1A}R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halo, haloalkyl having 1 to 3 halo groups, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Z is —C(O)— or —SO₂—; and
Y is CH₂, NH, or S; and
WSG is a water soluble group;
with the proviso that when Z is —C(O)—, then Y is not NH; when Y is CH₂, W¹ is O, N(CH₃), or CH₂ for formula I, then R² is not hydrogen or C₁₋₁₀ alkyl; and when Y is CH₂, W¹ is O, N(CH₃), or CH₂, and R³, R⁵, R⁶ are hydrogen for formula II, then R² is not hydrogen or C₁₋₁₀ alkyl.

This disclosure also provides pharmaceutical compositions comprising a compound of formula I or II, or a pharmaceutically acceptable salt, tautomer or a prodrug thereof as described herein. Also, provided are methods for determining whether a patient has a neurological disease or disorder comprising administering to the patient a compound of formula I or II as described herein, or a pharmaceutically acceptable salt, tautomer or a prodrug thereof, or a pharmaceutical composition thereof.

### BRIEF DESCRIPTION OF THE DRAWINGS

FIG. 1 presents the images of live retinal imaging of Tau in 3RTau transgenic mouse model following IV injection of compound 1.

FIG. 2 presents the images of live retinal imaging of Tau in 3RTau transgenic mouse model following IV injection of compound 1a.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (e.g., “-” or “—”) indicates a bond, which may be a point of attachment for a substituent. For example, —C(O)NH₂ is attached through the carbon atom. Chemical groups may be depicted with or without one or more dashes without losing their ordinary meaning. A wavy line drawn through a line in a structure indicates a point of attachment of a substituent or group.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl,” by itself or as part of another substituent, represent a straight (i.e. unbranched) or branched chain, or combination thereof, which may be fully saturated, mono- or polyunsaturated and can include di- and multivalent radicals, having the number of carbon atoms designated (e.g., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like.

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, and butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl ring, the resulting ring system is heteroaryl.

“Cyano” refers to —CN.

“Cycloalkyl” refers to a saturated or partially unsaturated cyclic alkyl, alkenyl, or alkynyl group having a single ring or multiple rings including fused, bridged, and spiro ring systems. Cycloalkyl also refers to ring systems including multiple carbocyclic rings fused together wherein one of the fused rings is an aromatic ring but the ring system is not fully aromatic. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, and cyclohexenyl.

The terms “halo” or “halogen,” by themselves or as part of another substituent, mean, unless otherwise stated, a fluorine, chlorine, bromine, or iodine atom.

“Haloalkyl” refers to an unbranched or branched alkyl group as defined above, wherein one or more hydrogen atoms are replaced by a halogen. For example, where an alkyl residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, respectively, which may or may not be the same halogen. Examples of haloalkyl include, but are not limited to, difluoromethyl (—CHF₂), trifluoromethyl (—CF₃), fluoromethyl, 2,2,2-trifluoroethyl, 4-chlorobutyl, and 3-bromopropyl.

“Heteroalkyl,” by itself or in combination with another term, means, unless otherwise stated, a straight or branched chain consisting of at least one carbon atom and at least one heteroatom selected from the group consisting of N, O, S, P, and Si, and wherein the N and S atoms may optionally be oxidized and the N may optionally be quaternized. The heteroatom(s) N, O, S, P, and Si, may be included at any non-terminal position of the heteroalkyl group or at the position at which the heteroalkyl group is attached. Two or more heteroatoms may be consecutive in the chain. Examples heteroalkyl include, but are not limited to, —CH₂—CH₂—O—CH₃, —CH₂—CH₂—O—CH₂—CH₂—O—CH₃, —CH₂—CH₂—O—CH₂—CH₂—O—CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, O—CH₃, and —O—CH₂—CH₃.

“Heteroaryl” refers to an aromatic group, including groups having an aromatic tautomer or resonance structure, having a single ring, multiple rings, or multiple fused rings, with one or more ring heteroatoms independently selected from N, O, and S, wherein the nitrogen and sulfur atoms are optionally oxidized, and the nitrogen atom(s) are optionally quaternized. As used herein, heteroaryl includes 3 to 20 ring atoms (i.e., 3- to 20-membered heteroaryl), 3 to 12 ring atoms (i.e., 3- to 12-membered heteroaryl), or 5 to 10 ring atoms (i.e., 5- to 10-membered heteroaryl), and 1 to 5 heteroatoms independently selected from N, O, and S. Heteroaryl does not encompass or overlap with aryl as defined above. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, triazolyl, 1,2,3-triazolyl, 1,2,4-triazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, 6-quinolyl, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Substituents for each of the above noted aryl and heteroaryl ring systems are selected from the group of acceptable substituents described below.

The term “heterocyclyl” means a cyclic versions of “heteroalkyl.” Additionally, for heterocyclyl, a heteroatom can occupy the position at which the heterocyclyl is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocyclyl include, but are not limited to, tetrahydropyran, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Examples of heterocyclyl include, but are not limited to glucose, mannose, allose, altrose, gulose, idose, galactose, and talose. Examples of heterocyclyl include, but are not limited to:

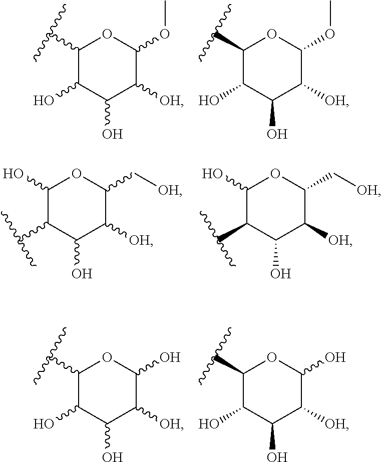

and the like.

“Hydroxyl” and “hydroxy” are used interchangeably and refer to —OH. “Oxo” refers to a double bonded O, written as, e.g., (═O) or (O). Where tautomeric forms of the compound exist, hydroxyl and oxo groups are interchangeable.

“Thiol” refers to —SH.

Each of the above terms (e.g., “alkyl,” “heteroalkyl,” “aryl,” and “heteroaryl”) may include both substituted and unsubstituted forms of the indicated radical.

As used herein, the term “heteroatom” or “ring heteroatom” is meant to include oxygen (O), nitrogen (N), sulfur (S), phosphorus (P), and silicon (Si).

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers, and carbonyl containing compounds may exist in equilibrium with enol tautomers. Regardless of which tautomer is shown, and regardless of the nature of the equilibrium among tautomers, the compounds are understood by one of ordinary skill in the art to comprise all tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and¹²⁵I. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes deuterated analogs of compounds of Formula I or II in which from 1 to n hydrogens attached, e.g., to a carbon atom is/are replaced by deuterium, in which n is the number of hydrogens in the molecule. Such compounds may exhibit increased resistance to metabolism and may be useful for increasing the half-life of any compound of Formula I or II when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted compounds of the disclosure may have improved DMPK (drug metabolism and pharmacokinetics) properties, relating to distribution, metabolism and excretion (ADME). Substitution with heavier isotopes such as deuterium may afford certain advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In some embodiments, the compounds are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with organic acids. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), alkenyl amines (i.e., NH₂(alkenyl)), dialkenyl amines (i.e., HN(alkenyl)₂), trialkenyl amines (i.e., N(alkenyl)₃), mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed alkyl, alkenyl, cycloalkyl, and/or aryl amines. Specific examples of suitable amines include, by way of example only, diisopropylamine, triethyl amine, diethyl amine, tri(iso-propyl)amine, tri(n-propyl)amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. Unless otherwise stated, the one or more substituents may be any substituent provided herein, or a combination thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. The term “substituted alkyl” is not meant to include “haloalkyl,” which is described herein.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceutical compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

“Prodrug” refers to any compound that when administered to a biological system generates a parent compound, as a result of spontaneous chemical reaction(s), enzyme catalyzed chemical reaction(s), photolysis, and/or metabolic chemical reaction(s). A prodrug is thus a covalently modified analog or latent form of a biologically active parent compound.

“Water soluble group” or “WSG” refers to any group that alters the solubility of the compounds of formula I or II in water. The examples include, but are not limited to sugars, polyethylene glycol, polypropylene glycol, co-polymer of polyethylene glycol and polypropylene glycol or alkoxy derivatives thereof. Other examples include compounds of formula:

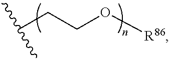

wherein n is an integer from 1-50 and R⁸⁶ is H, C₁₋₁₀ alkyl, C₁₋₁₀ heteroalkyl, C₃₋₁₀ cycloalkyl, C₁₋₁₀ heterocyclyl, C₆₋₁₀ aryl, or C₁₋₁₀ heteroaryl, each of which except for hydrogen is optionally substituted with one or more C₁₋₁₀ alkyl, C₁₋₁₀ haloalkyl, C₁₋₁₀ heteroalkyl, C₃₋₁₀ cycloalkyl, C₁₋₁₀ heterocyclyl, C₆₋₁₀ aryl, or C₁₋₁₀ heteroaryl. In some embodiments, R⁸⁶ is a prodrug moiety. The examples of prodrugs include, but are not limited to, phosphate prodrugs.

### Compounds

The present disclosure provides compounds useful in the detection and treatment of neurological diseases and disorders.

In some embodiments, this disclosure provides a compound of formula I or II:

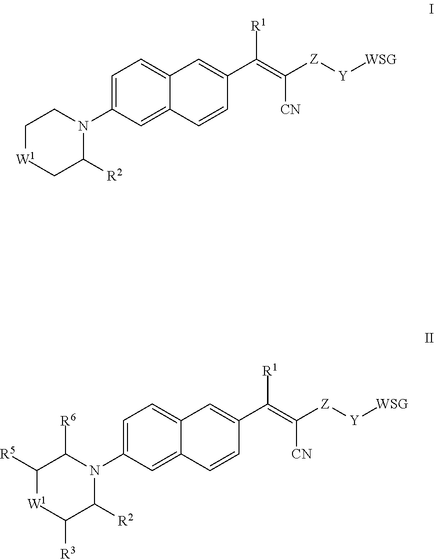

or a pharmaceutically acceptable salt, tautomer or a prodrug thereof wherein:
W¹ is O, N(R¹⁴), or C(R⁴)₂;
R¹⁴ is hydrogen, alkyl, halomethyl having 1 to 3 halo groups, —COOH, —CONH₂, substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is selected from hydrogen, halo, haloalkyl having 1 to 3 halo groups, or cyano;
R², R³, R⁴, R⁵, and R⁶ are each independently hydrogen, halo, haloalkyl having 1 to 3 halo groups, —CN, —C(O)R^{1A}, —C(O)OR^{1A}, —C(O)NR^{1A}R^{1B}, —OR^{1A}, —NHC(O)NR^{1A}R^{1B}, —NR^{1A}R^{1B}, —NHNR^{1A}R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halo, haloalkyl having 1 to 3 halo groups, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Z is —C(O)— or —SO₂—; and
Y is CH₂, NH, or S; and
WSG is a water soluble group;
with the proviso that when Z is —C(O)—, then Y is not NH; when Y is CH₂, W¹ is O, N(CH₃), or CH₂ for formula I, then R² is not hydrogen or C₁₋₁₀ alkyl; and when Y is CH₂, W¹ is O, N(CH₃), or CH₂, and R³, R⁵, R⁶ are hydrogen for formula II, then R² is not hydrogen or C₁₋₁₀ alkyl.

In some embodiments, this disclosure provides a compound of formula I or II:

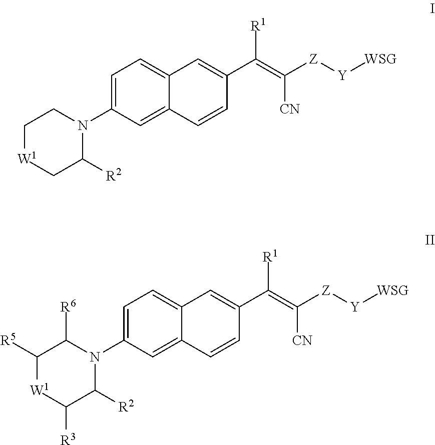

or a pharmaceutically acceptable salt, tautomer or a prodrug thereof.

W¹ is O, N(R¹⁴), or C(R⁴)₂.

R¹⁴ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCI₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹, R², R³, R⁴, R⁵, and R⁶ are each independently hydrogen, halo, —CX¹₃, —CHX¹₂, —CH₂X¹, —OCX¹₃, —OCH₂X¹, —OCHX¹₂, —SOᵥ₁R^{1A}, —SOᵥ₁NR^{1A}R^{1B}, —CN, —C(O)R^{1A}, —C(O)OR^{1A}, —C(O)NR^{1A}R^{1B}, —OR^{1A}, —ONR^{1A}R^{1B}, —NHC(O)NR^{1A}R^{1B}, —N(O)ₘ₁, —NR^{1A}R^{1B}, —NHNR^{1A}R^{1B}, —NR^{1A}SO₂R^{1B}, —NR^{1A}C(O)R^{1B}, —NR^{1A}C(O)OR^{1B}, —NR^{1A}OR^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} are each independently hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹ is —F, —Cl, —Br, or —I.

n1 is independently an integer from 0 to 4.

m1 is independently 1 or 2.

v1 is independently 1 or 2.

Z is —C(O)— or —SO₂—;

Y is CH₂, NH, or S.

WSG is a water soluble group.

In some embodiments, when Z is —C(O)—, then Y is not NH.

In some embodiments, this disclosure provides a compound of formula I or II as described herein, wherein Z is —C(O)—. In some embodiments, Y is CH₂. In some embodiments, Y is S.

In some embodiments, this disclosure provides the compound of formula I or II:

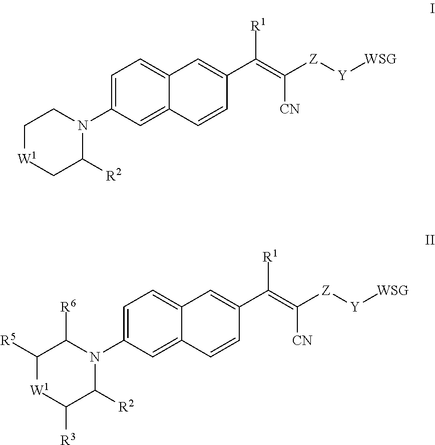

or a pharmaceutically acceptable salt, tautomer or a prodrug thereof.

W¹ is O, N(R¹⁴), or C(R⁴)₂.

R¹⁴ is hydrogen, alkyl, halomethyl having 1 to 3 halo groups, —COOH, —CONH₂, substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is selected from hydrogen, halo, haloalkyl having 1 to 3 halo groups, or cyano.

R², R³, R⁴, R⁵, and R⁶ are each independently hydrogen, halo, haloalkyl having 1 to 3 halo groups, —CN, —C(O)R^{1A}, —C(O)OR^{1A}, —C(O)NR^{1A}R^{1B}, —OR^{1A}, —NHC(O)NR^{1A}R^{1B}, —NR^{1A}R^{1B}, —NHNR^{1A}R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} are each independently hydrogen, halo, haloalkyl having 1 to 3 halo groups, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Z is —C(O)— or —SO₂—.

Y is CH₂, NH, or S.

WSG is a water soluble group selected from the group consisting of:

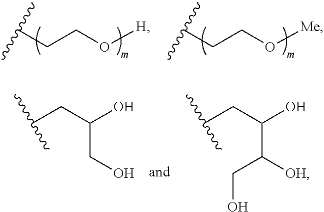

wherein m is an integer having a value from 1-10.

In some embodiments, when Z is —C(O)—, then Y is not NH.

In some embodiments, when Y is CH₂, W¹ is O, N(CH₃), or CH₂ for formula I, then R² is not hydrogen or C₁₋₁₀ alkyl.

In some embodiments, when Y is CH₂, W¹ is O, N(CH₃), or CH₂, and R³, R⁵, R⁶ are hydrogen for formula II, then R² is not hydrogen or C₁₋₁₀ alkyl.

In some embodiments, this disclosure provides a compound of formula I or II as described herein, wherein Z is —C(O)—. In some embodiments, Y is CH₂. In some embodiments, Y is S.

In some embodiments, this disclosure provides a compound of formula I:

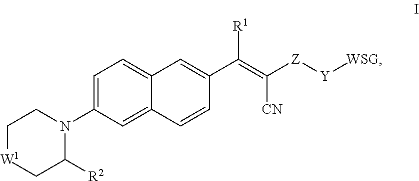

or a pharmaceutically acceptable salt, tautomer, or prodrug thereof. WSG, W¹, R¹, R², Z, and Y are as described herein.

In some embodiments, this disclosure provides a compound of formula Ia:

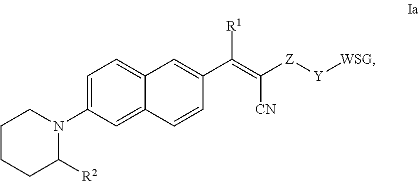

or a pharmaceutically acceptable salt, tautomer, or prodrug thereof. WSG, R¹, R², Z, and Y are as described herein.

In some embodiments, this disclosure provides a compound of formula Ib:

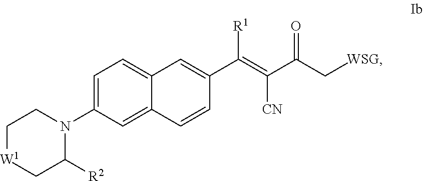

or a pharmaceutically acceptable salt, tautomer, or prodrug thereof. WSG, W¹, R¹, and R² are as described herein.

In some embodiments, this disclosure provides a compound of formula Ic:

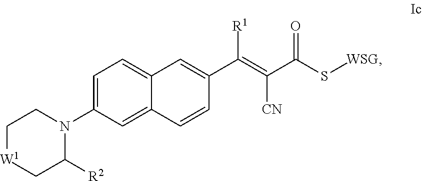

or a pharmaceutically acceptable salt, tautomer, or prodrug thereof. WSG, W¹, R¹, and R² are as described herein.

In some embodiments, this disclosure provides a compound of formula Id:

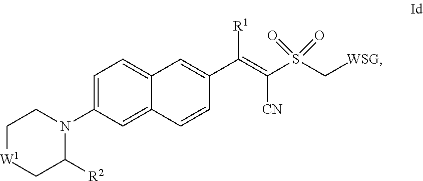

or a pharmaceutically acceptable salt, tautomer, or prodrug thereof. WSG, W¹, R¹, and R² are as described herein.

In some embodiments, this disclosure provides a compound of formula Ie:

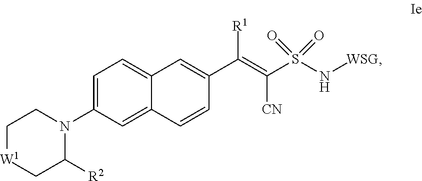

or a pharmaceutically acceptable salt, tautomer, or prodrug thereof. WSG, W¹, R¹, and R² are as described herein.

In some embodiments, this disclosure provides a compound of formula If:

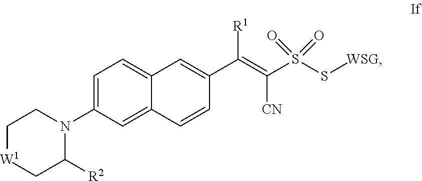

or a pharmaceutically acceptable salt, tautomer, or prodrug thereof. WSG, W¹, R¹, and R² are as described herein.

In some embodiments, R¹ is hydrogen. In some embodiments, R² is hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, —CN, —OH, —NH₂, —COOH, —CONH₂, —NHNH₂, —NHC(O)NHNH₂, substituted or unsubstituted C₁-C₆ alkyl, or substituted or unsubstituted 2 to 6 membered heteroalkyl.

In some embodiments, this disclosure provides a compound of formula II:

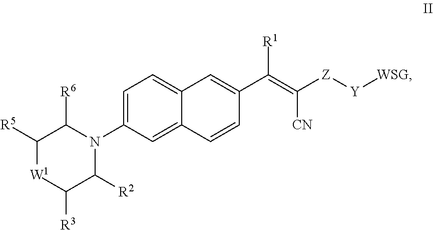

or a pharmaceutically acceptable salt, tautomer, or prodrug thereof. WSG, W¹, R¹, R², R³, R⁵, R⁶, Z, and Y are as described herein.

In some embodiments, this disclosure provides a compound of formula IIa:

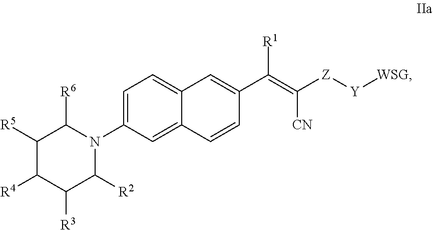

or a pharmaceutically acceptable salt, tautomer, or prodrug thereof. WSG, R¹, R², R³, R⁴, R⁵, R⁶, Z, and Y are as described herein.

In some embodiments, this disclosure provides a compound of formula IIb:

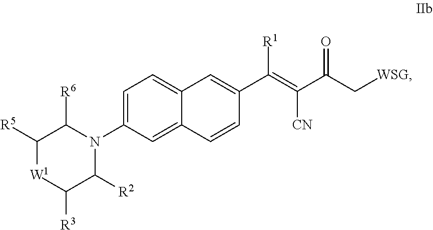

or a pharmaceutically acceptable salt, tautomer, or prodrug thereof. WSG, W¹, R¹, R², R³, R⁵, and R⁶ are as described herein.

In some embodiments, this disclosure provides a compound of formula IIc:

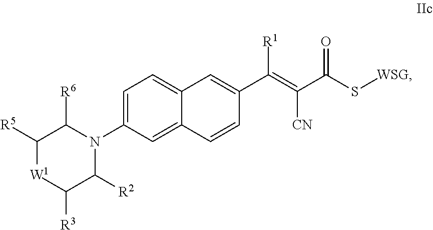

or a pharmaceutically acceptable salt, tautomer, or prodrug thereof. WSG, W¹, R¹, R², R³, R⁵, and R⁶ are as described herein.

In some embodiments, this disclosure provides a compound of formula IId:

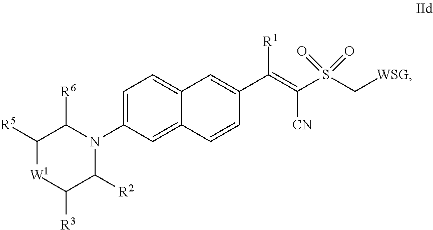

or a pharmaceutically acceptable salt, tautomer, or prodrug thereof. WSG, W¹, R¹, R², R³, R⁵, and R⁶ are as described herein.

In some embodiments, this disclosure provides a compound of formula IIe:

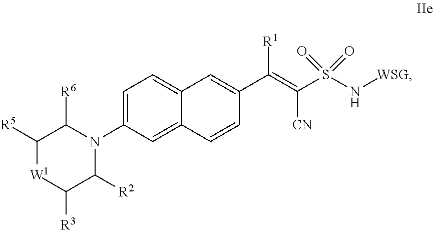

or a pharmaceutically acceptable salt, tautomer, or prodrug thereof. WSG, W¹, R¹, R², R³, R⁵, and R⁶ are as described herein.

In some embodiments, this disclosure provides a compound of formula IIf:

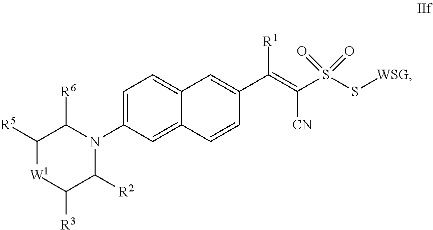

or a pharmaceutically acceptable salt, tautomer, or prodrug thereof. WSG, W¹, R¹, R², R³, R⁵, and R⁶ are as described herein.

In some embodiments, R¹ is hydrogen, halo, —CF₃, —CBr₃, —CCI₃, —CCl₃, —CHF₂, —CHBr₂, —CHCl₂, —CHI₂, —CHF, —CH₂Br, —CH₂Cl, —CH₂I, or —CN. In some embodiments, R¹ is —CF₃ or unsubstituted methyl. In some embodiments, R², R³, R⁴, R⁵, and R⁶ are independently hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, —CN, —OH, —NH₂, —COOH, —CONH₂, substituted or unsubstituted C₁-C₆ alkyl, or substituted or unsubstituted 2 to 6 membered heteroalkyl. In some embodiments, R², R³, R⁴, R⁵, and R⁶ are hydrogen.

In some embodiments, this disclosure provides a compound of formula I or II as described herein, wherein WSG is selected from the group consisting of:

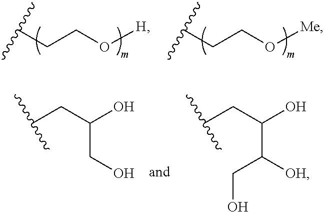

wherein m is an integer having a value from 1-10.

In some embodiments, this disclosure provides a compound of formula I or II as described herein, wherein WSG is selected from the group consisting of:

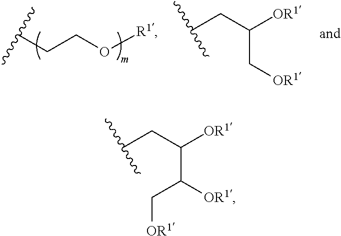

wherein m is an integer having a value from 1-10 and each R^{1′} is independently selected from the group consisting of:
H,

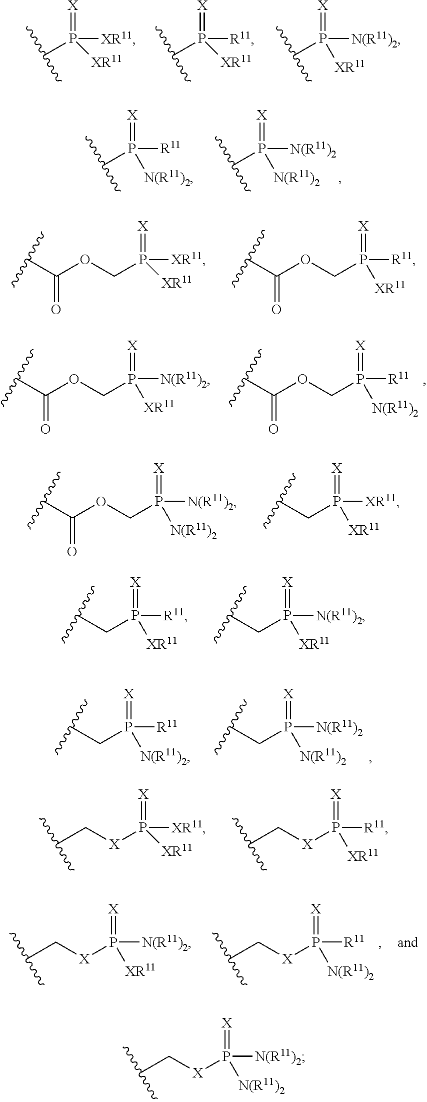

wherein each X is independently O or S;
each R¹¹ is independently selected from hydrogen, C₁₋₁₀ alkyl, C₂₋₆ alkenyl, C₂₋₆ alkynyl, C₃₋₁₀ cycloalkyl, C₆₋₁₀ aryl, 5- to 10-membered heteroaryl and 4- to 10-membered heterocyclyl; wherein alkyl, alkenyl, alkynyl, cycloalkyl, aryl, heteroaryl and heterocyclyl are optionally substituted with one to four R²¹; or each XR¹¹ may independently be —XP(X)(R¹²)₂;
each R¹² is independently selected from hydroxy, thiol, —XP(X)(R¹³)₂, C₁₋₁₀ alkyl, —O—C₁₋₁₀ alkyl, and —S—C₁₋₁₀ alkyl;
each R¹³ is independently selected from hydroxy, thiol, C₁₋₁₀ alkyl, —O—C₁₋₁₀ alkyl, and —S—C₁₋₁₀ alkyl;
each R²¹ is independently selected from halo, hydroxy, thiol, —NO₂, —N₃, cyano, C₁₋₁₀ alkyl, C₂₋₆ alkenyl, C₂₋₆ alkynyl, C₃₋₁₀ cycloalkyl, C₁₋₈ haloalkyl, C₆₋₁₀ aryl, 5- to 10-membered heteroaryl, 4- to 10-membered heterocyclyl, —O—C₁₋₁₀ alkyl, —O—C₂₋₆ alkenyl, —O—C₂₋₆ alkynyl, —O—C₃₋₁₀ cycloalkyl, —O—C₁₋₈ haloalkyl, —O-aryl, —O-heteroaryl, —O-heterocyclyl, —NH₂, —NH(R³¹), —N(R³¹)₂, —C(O)(R³¹), —C(O)O(R³¹), —C(O)OH, —C(O)NH₂, —C(O)NH(R³¹), —C(O)N(R³¹)₂, —NHC(O)(R³¹), —NHC(O)O(R³¹), —NHC(O)NH(R³¹), —S(R³¹), —NHS(O)_{y}(R³¹), —N(C₁₋₁₀ alkyl)S(O)_{y}(R³¹), —S(O)_{y}N(R³¹)₂, —S(O)NH(R³¹), and —S(O)_{y}(R³¹);
each R³¹ is independently selected from C₁₋₁₀ alkyl, C₂₋₆ alkenyl, C₂₋₆ alkynyl, C₃₋₁₀ cycloalkyl, C₁₋₈ haloalkyl, aryl, heteroaryl, and heterocyclyl; and
each y is independently 1 or 2.

In some embodiments, this disclosure provides a compound selected from the group consisting of:

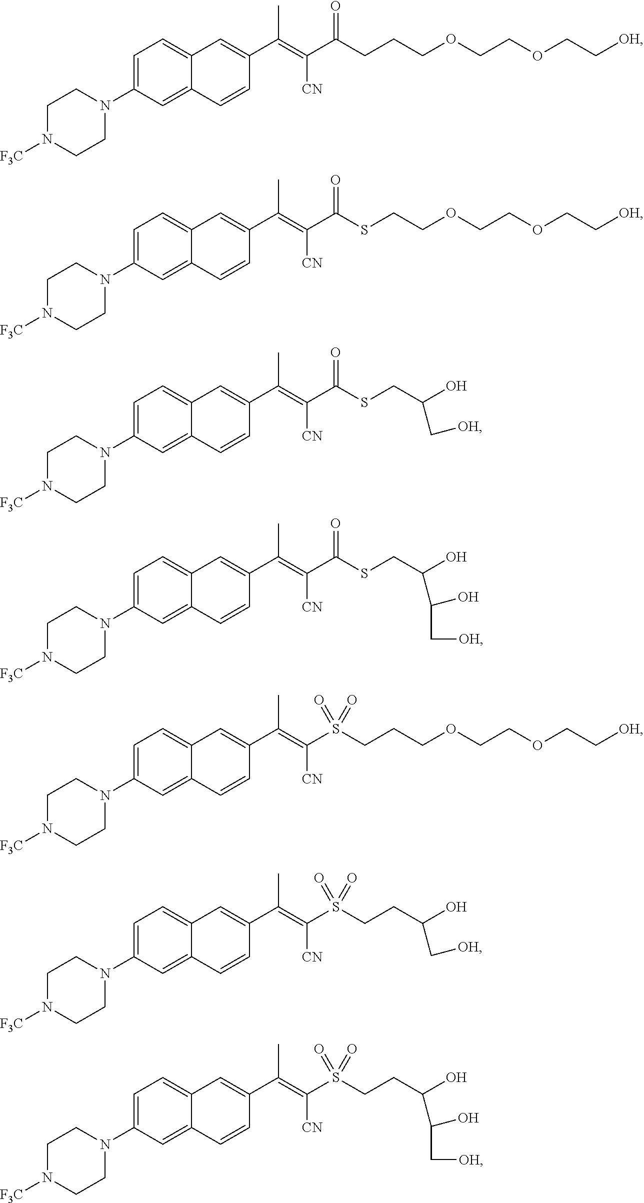

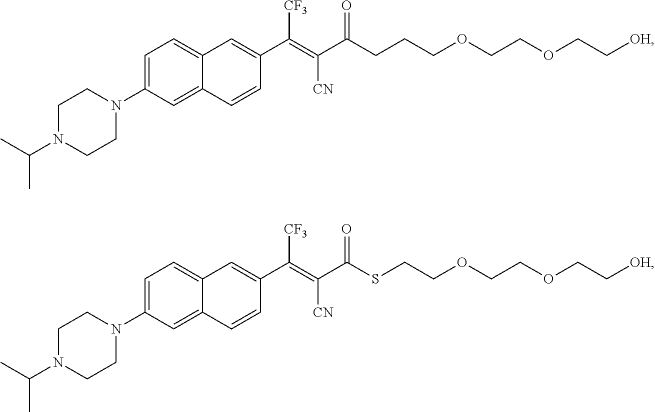 [SMILES: CC(C)N1CCN(c2ccc3cc(/C(=C(\C#N)C(=O)CCCOCCOCCO)C(F)(F)F)ccc3c2)CC1 ; CC(C)N1CCN(c2ccc3cc(/C(=C(\C#N)C(=O)SCCOCCOCCO)C(F)(F)F)ccc3c2)CC1]

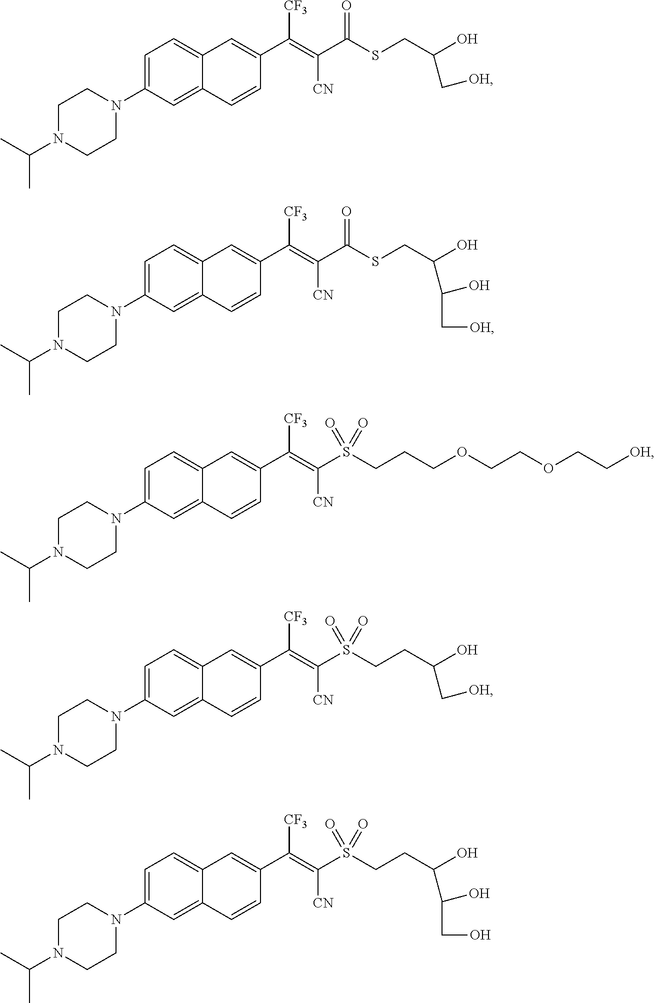 [SMILES: CC(C)N1CCN(c2ccc3cc(/C(=C(\C#N)C(=O)SCC(O)CO)C(F)(F)F)ccc3c2)CC1 ; CC(C)N1CCN(c2ccc3cc(/C(=C(\C#N)C(=O)SCC(O)C(O)CO)C(F)(F)F)ccc3c2)CC1 ; CC(C)N1CCN(c2ccc3cc(/C(=C(\C#N)S(=O)(=O)CCCOCCOCCO)C(F)(F)F)ccc3c2)CC1 ; CC(C)N1CCN(c2ccc3cc(/C(=C(\C#N)S(=O)(=O)CCC(O)CO)C(F)(F)F)ccc3c2)CC1 ; CC(C)N1CCN(c2ccc3cc(/C(=C(\C#N)S(=O)(=O)CCC(O)C(O)CO)C(F)(F)F)ccc3c2)CC1]

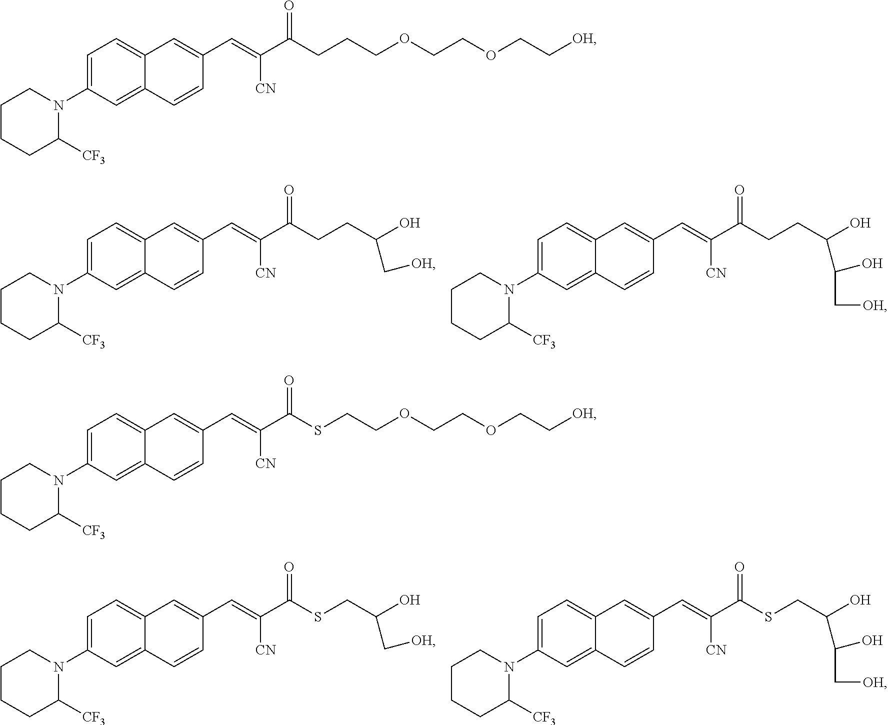

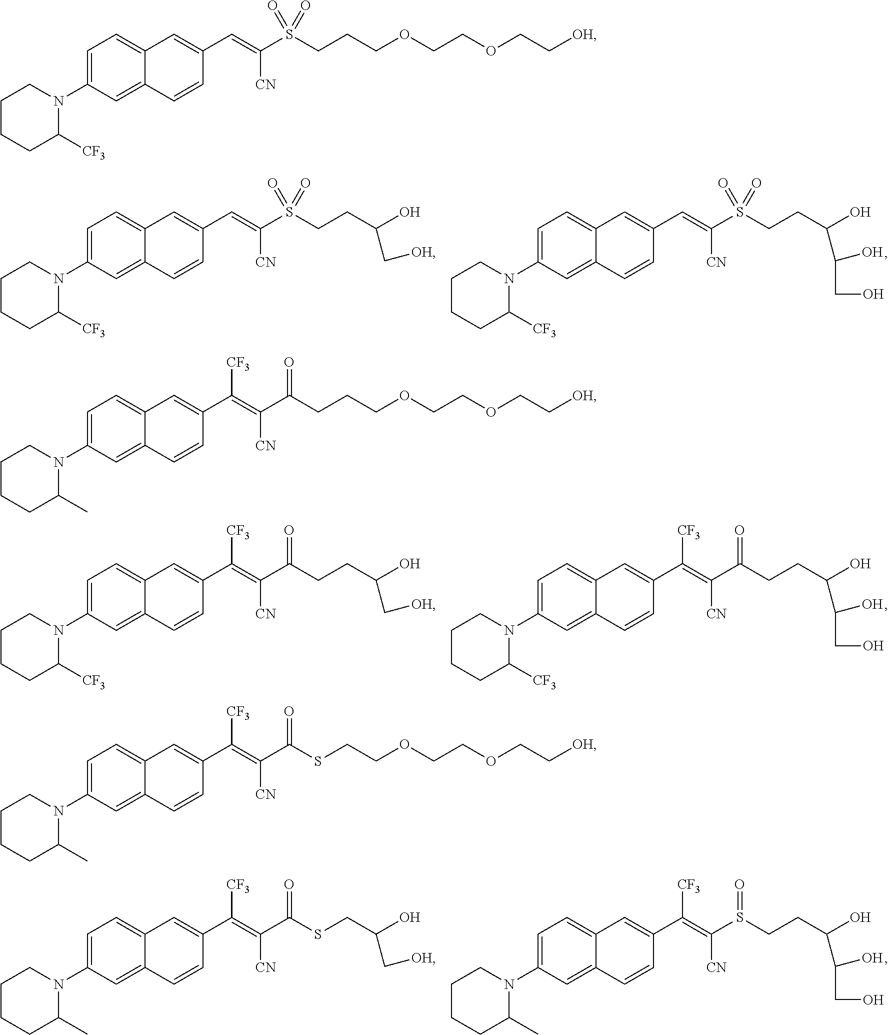

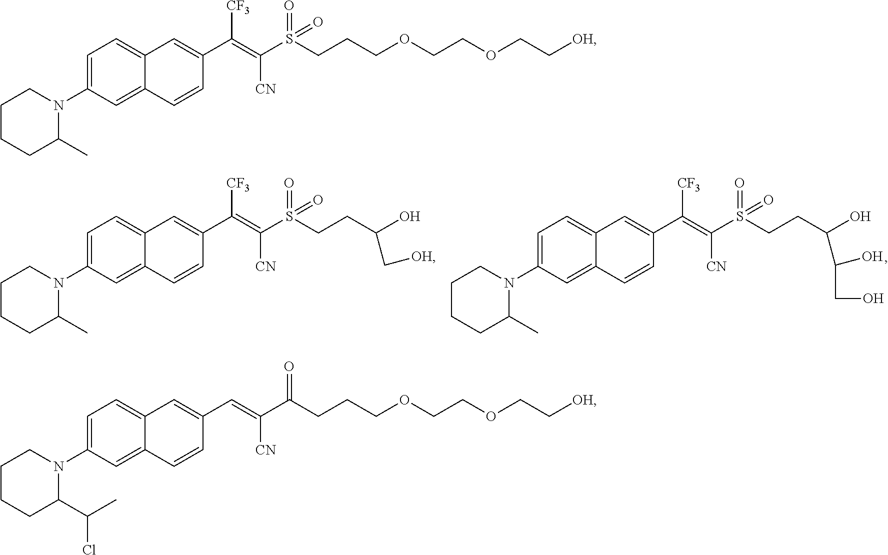

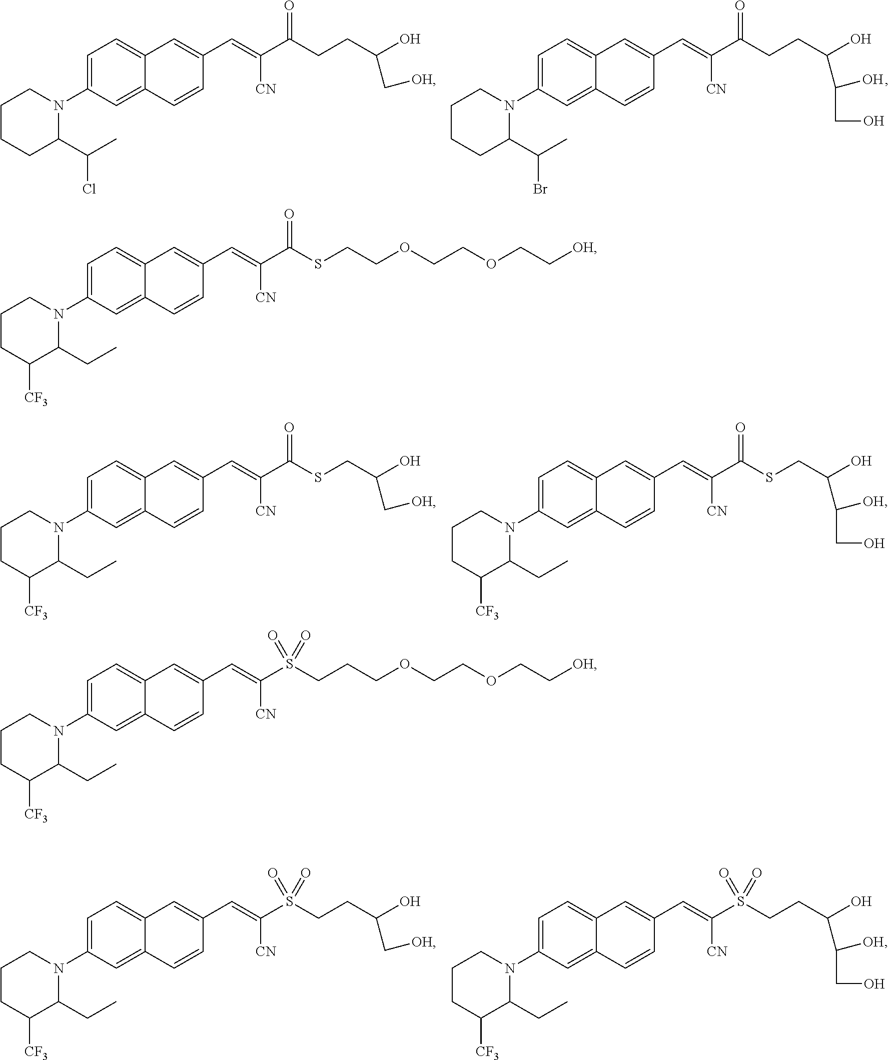

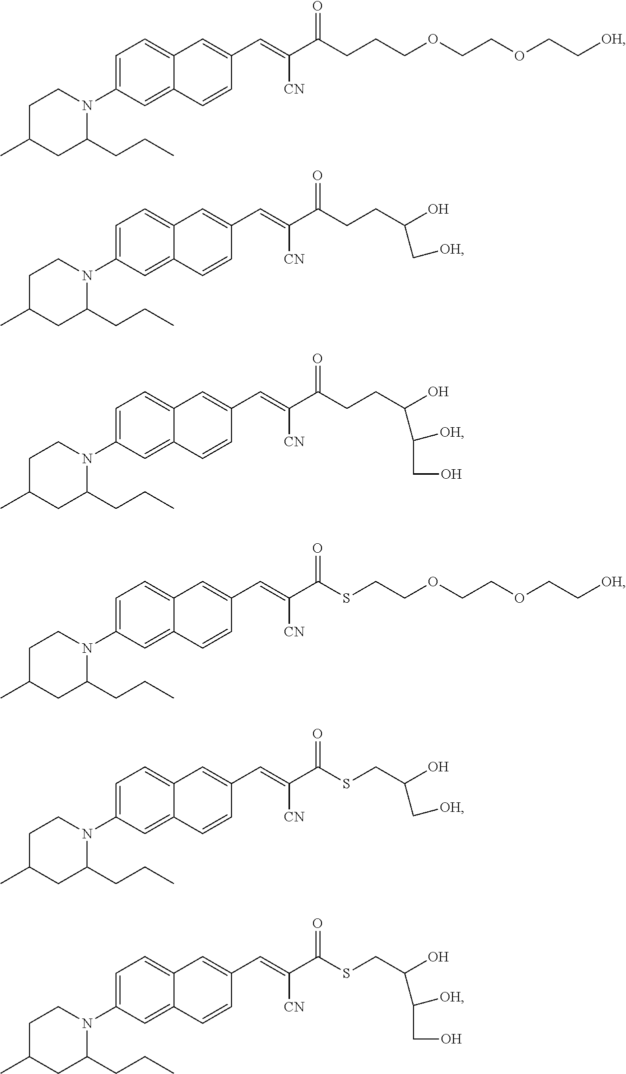 [SMILES: CCCC1CC(C)CCN1c1ccc2cc(/C=C(\C#N)C(=O)CCCOCCOCCO)ccc2c1 ; CCCC1CC(C)CCN1c1ccc2cc(/C=C(\C#N)C(=O)CCC(O)CO)ccc2c1 ; CCCC1CC(C)CCN1c1ccc2cc(/C=C(\C#N)C(=O)CCC(O)C(O)CO)ccc2c1 ; CCCC1CC(C)CCN1c1ccc2cc(/C=C(\C#N)C(=O)SCCOCCOCCO)ccc2c1 ; CCCC1CC(C)CCN1c1ccc2cc(/C=C(\C#N)C(=O)SCC(O)CO)ccc2c1 ; CCCC1CC(C)CCN1c1ccc2cc(/C=C(\C#N)C(=O)SCC(O)C(O)CO)ccc2c1]

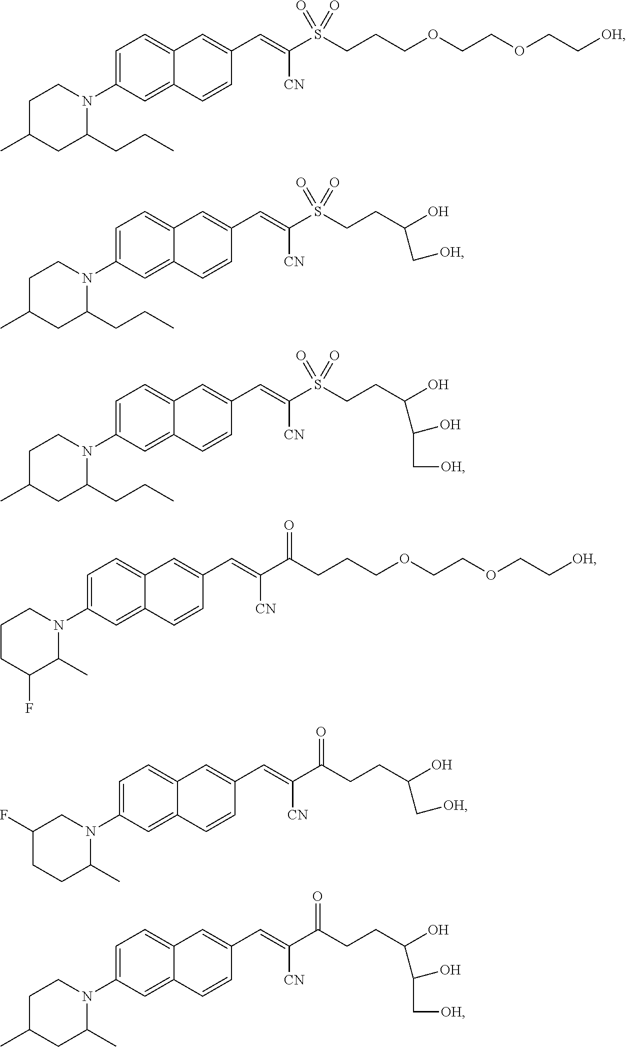 [SMILES: CCCC1CC(C)CCN1c1ccc2cc(/C=C(\C#N)S(=O)(=O)CCCOCCOCCO)ccc2c1 ; CCCC1CC(C)CCN1c1ccc2cc(/C=C(\C#N)S(=O)(=O)CCC(O)CO)ccc2c1 ; CCCC1CC(C)CCN1c1ccc2cc(/C=C(\C#N)S(=O)(=O)CCC(O)C(O)CO)ccc2c1 ; CC1C(F)CCCN1c1ccc2cc(/C=C(\C#N)C(=O)CCCOCCOCCO)ccc2c1 ; CC1CCC(F)CN1c1ccc2cc(/C=C(\C#N)C(=O)CCC(O)CO)ccc2c1 ; CC1CCN(c2ccc3cc(/C=C(\C#N)C(=O)CCC(O)C(O)CO)ccc3c2)C(C)C1]

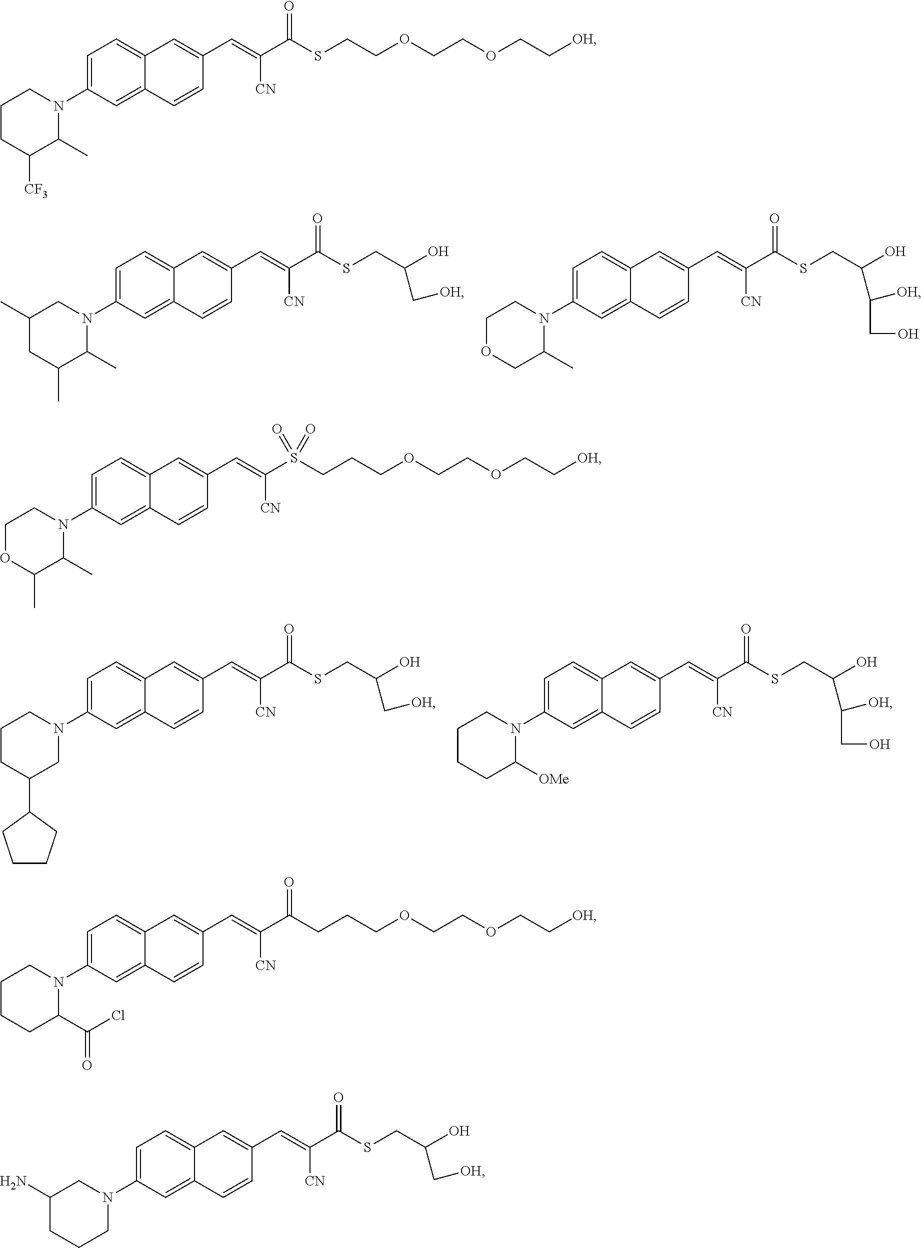

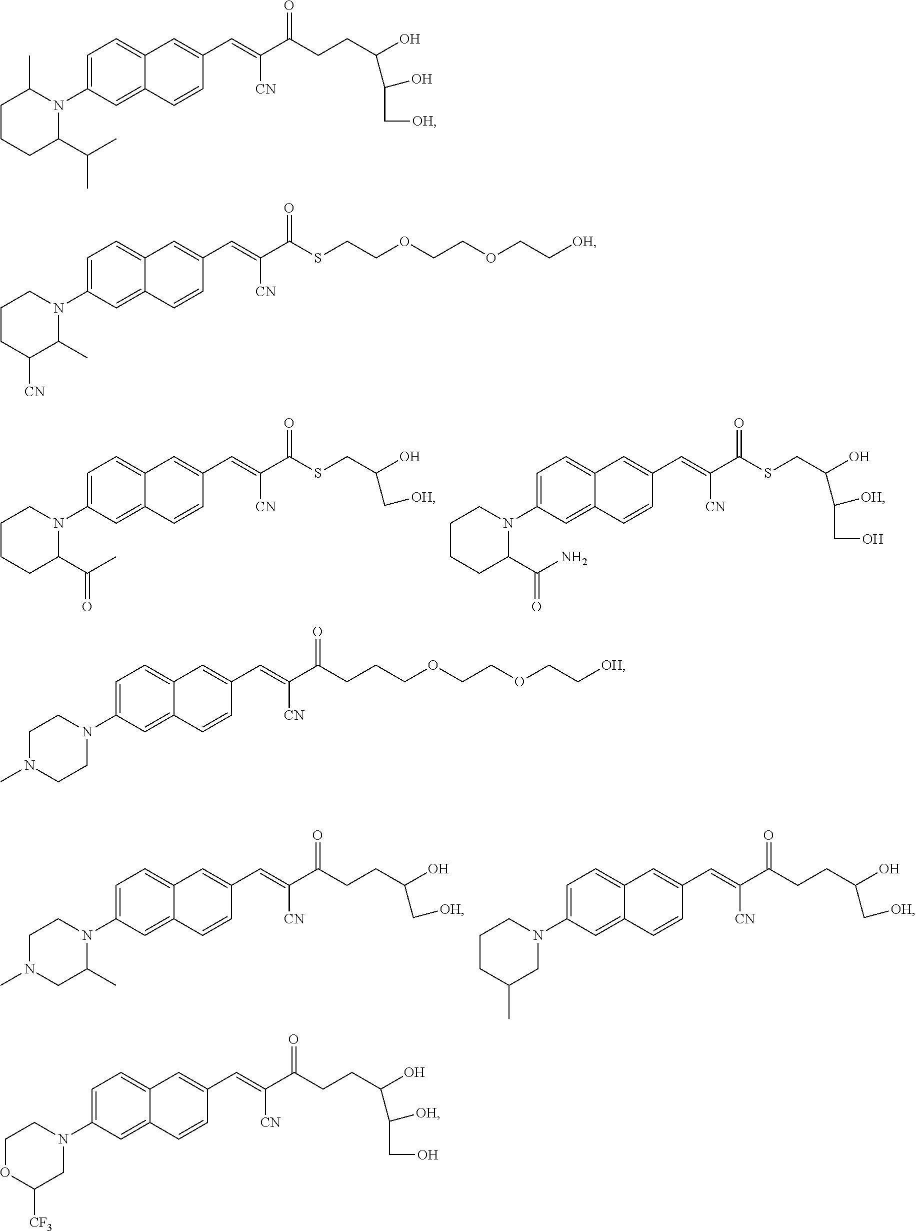

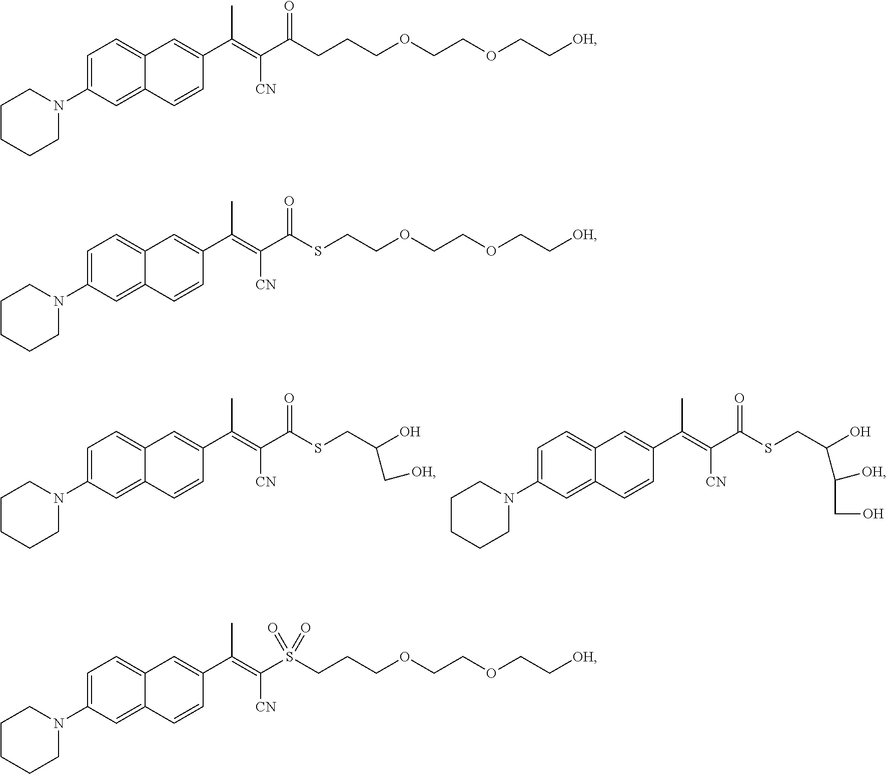

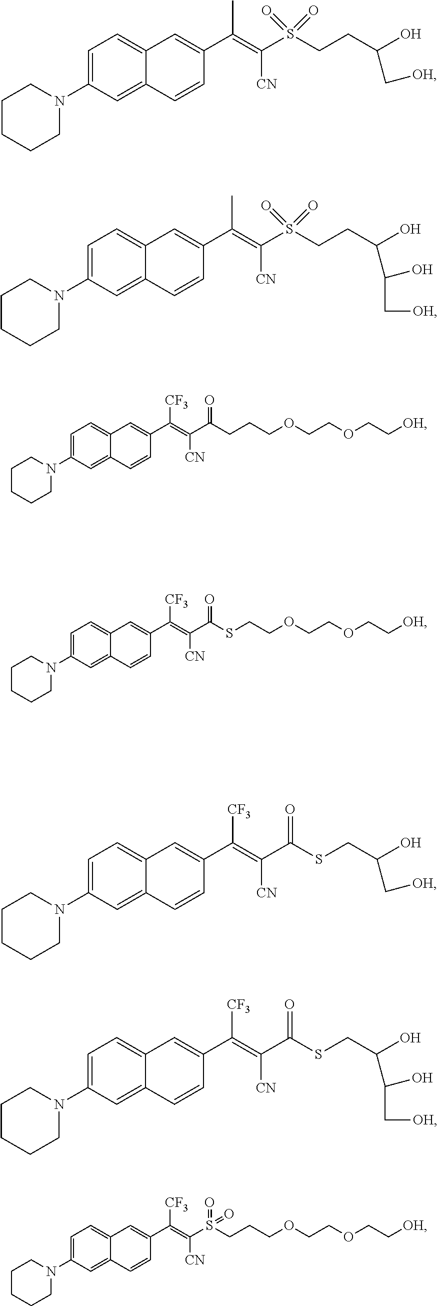

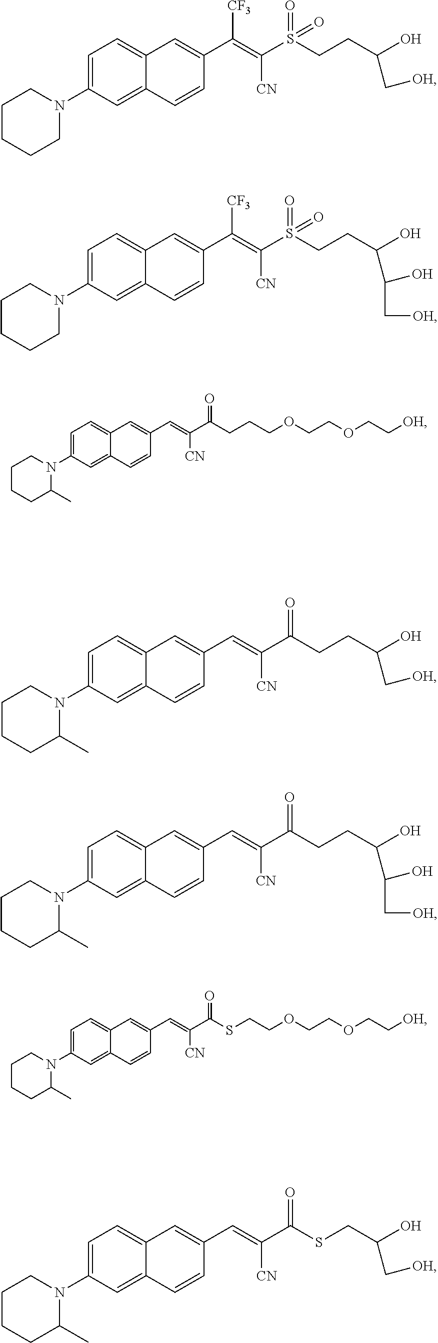

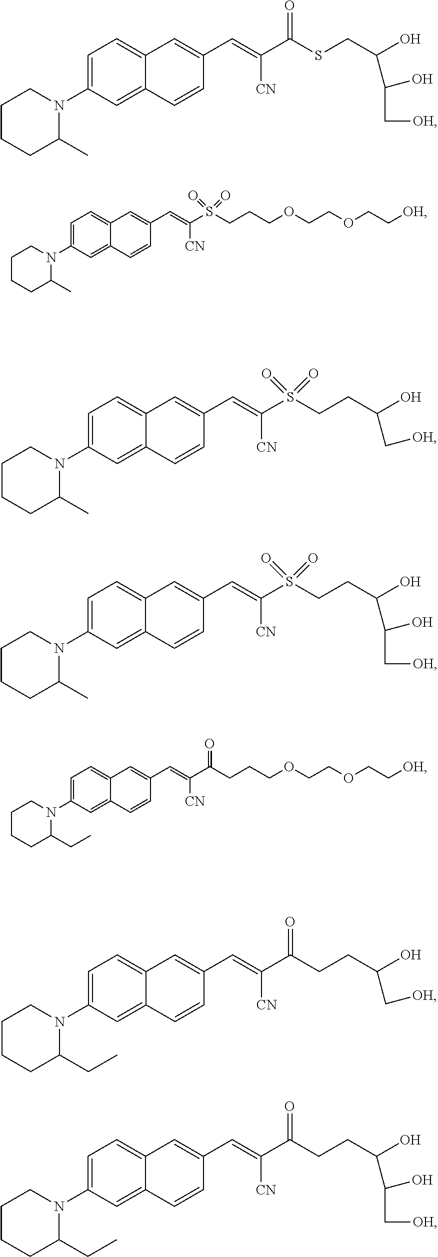

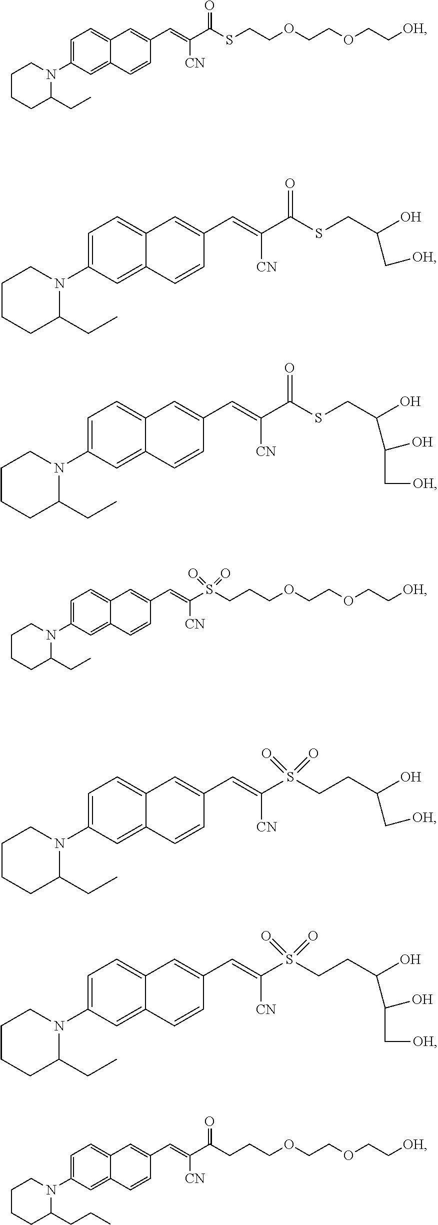

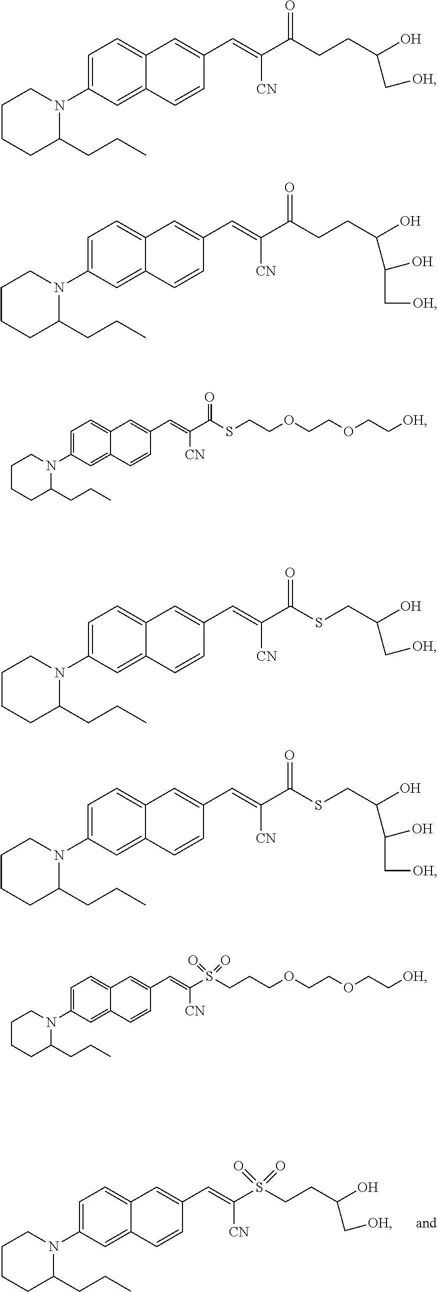

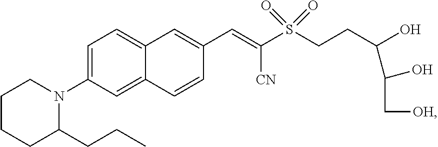 [SMILES: CCCC1CCCCN1c1ccc2cc(/C=C(\C#N)S(=O)(=O)CCC(O)C(O)CO)ccc2c1]

or a pharmaceutically acceptable salt, tautomer, or prodrug thereof.

In some embodiments, this disclosure provides the compound of formula:

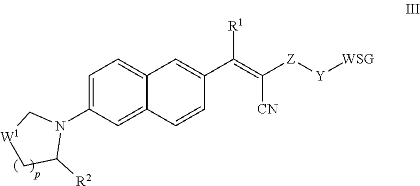

or a pharmaceutically acceptable salt, tautomer or a prodrug thereof.

p is 0 or 1.

W¹ is a bond, O, N(R¹⁴), C(R⁴)₂, CH₂—O—, CH₂—N(R¹⁴), or CH₂—C(R⁴)₂.

R¹⁴ is hydrogen, alkyl, halomethyl having 1 to 3 halo groups, —COOH, —CONH₂, substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is selected from hydrogen, halo, haloalkyl having 1 to 3 halo groups, or cyano.

R² is selected from hydrogen, halo, haloalkyl having 1 to 3 halo groups, —CN, —C(O)R^{1A}, —C(O)OR^{1A}, —C(O)NR^{1A}R^{1B}, —OR^{1A}, —NHC(O)NR^{1A}R^{1B}, —NR^{1A}R^{1B}, —NHNR^{1A}R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} are each independently hydrogen, halo, haloalkyl having 1 to 3 halo groups, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Z is —C(O)— or —SO₂—.

Y is CH₂, NH, or S.

WSG is a water soluble group.

In some embodiments, when Z is —C(O)—, then Y is not NH.

In some embodiments, when Y is CH₂, W¹ is O, N(CH₃), or CH₂ for formula I, then R² is not hydrogen or C₁₋₁₀ alkyl.

In some embodiments, when Y is CH₂, W¹ is O, N(CH₃), or CH₂, and R³, R⁵, R⁶ are hydrogen for formula II, then R² is not hydrogen or C₁₋₁₀ alkyl.

In some embodiments, this disclosure provides a compound of formula I or II as described herein, wherein Z is —C(O)—. In some embodiments, Y is CH₂. In some embodiments, Y is S.

It is contemplated that ketone compounds of formula IIa would provide increase fluorescence at excitation wavelength at 450 and/or 488 nm, and would have fewer metabolites which may cause adverse effects.

### General Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of formula I or II, e.g., compounds having structures described by, e.g., formula I or II or compounds disclosed herein, or a pharmaceutically acceptable salt thereof, may be accomplished as described in the examples and as known in the art.

Typical embodiments of compounds in accordance with the present disclosure may be synthesized using the reaction schemes and/or examples described below. It will be apparent given the description herein that the schemes may be altered by substitution of the materials with other materials having similar structures to result in products that are correspondingly different. Descriptions of syntheses follow to provide examples of how the steps may vary to provide desired products. Group labels (e.g., R¹) used in the reaction schemes herein are for illustrative purposes only and unless otherwise specified do not necessarily match by name or function the labels used elsewhere to describe compounds of formula I or II, or aspects or fragments thereof.

It will be appreciated that where process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

The materials and reagents for the following reactions are generally known compounds or can be prepared by known procedures or obvious modifications thereof. For example, many of the starting materials are available from commercial suppliers such as Aldrich Chemical Co. (Milwaukee, Wisconsin, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme 1 shows an exemplary synthetic route for the synthesis of compounds provided herein (e.g., compounds of Formula I or II).

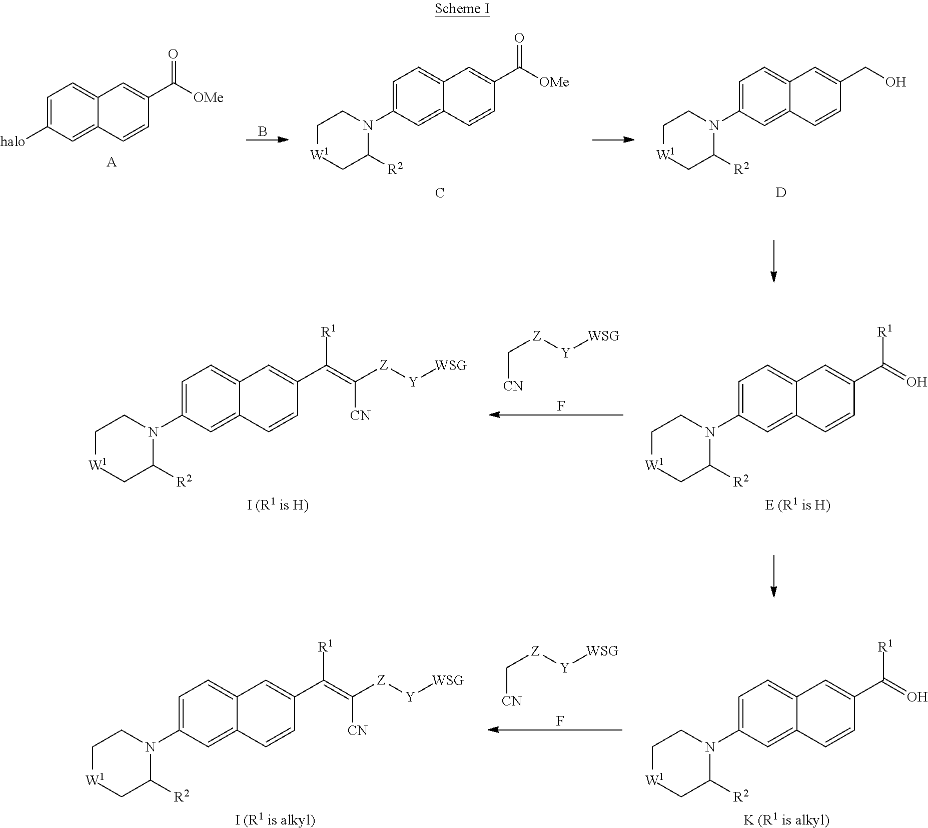

Halo substituted naphthalene, compound A, is coupled with piperidine compound B under suitable reaction conditions such as palladium catalyst and a base such as Cs₂CO₃ in a solvent such as toluene to provide piperidine substituted naphthalene, compound C. Reduction of the ester in compound C under suitable reaction conditions such as using a metal hydride reagent in a solvent such as THE provides compound D. Oxidation of compound D under suitable reaction conditions such as MnO₂ provides compound E (wherein R¹ is H). Coupling of compound E with compound F under suitable reaction conditions in presence of a base such as piperidine in a suitable solvent such as THF provides compounds of formula I or II, wherein R¹ is H. Grignard reaction of compound E (wherein R¹ is H) with a suitable alkyl magnesium halide in a solvent such as THF under suitable reaction conditions provides compound K (wherein R¹ is alkyl). Coupling of compound K with compound F under suitable reaction conditions in presence of a base such as piperidine in a suitable solvent such as THF provides compounds of formula I or II, wherein R¹ is alkyl.

Alternatively, the piperidine group can be added to the naphthalene after the aldehyde formation as shown in scheme II below.

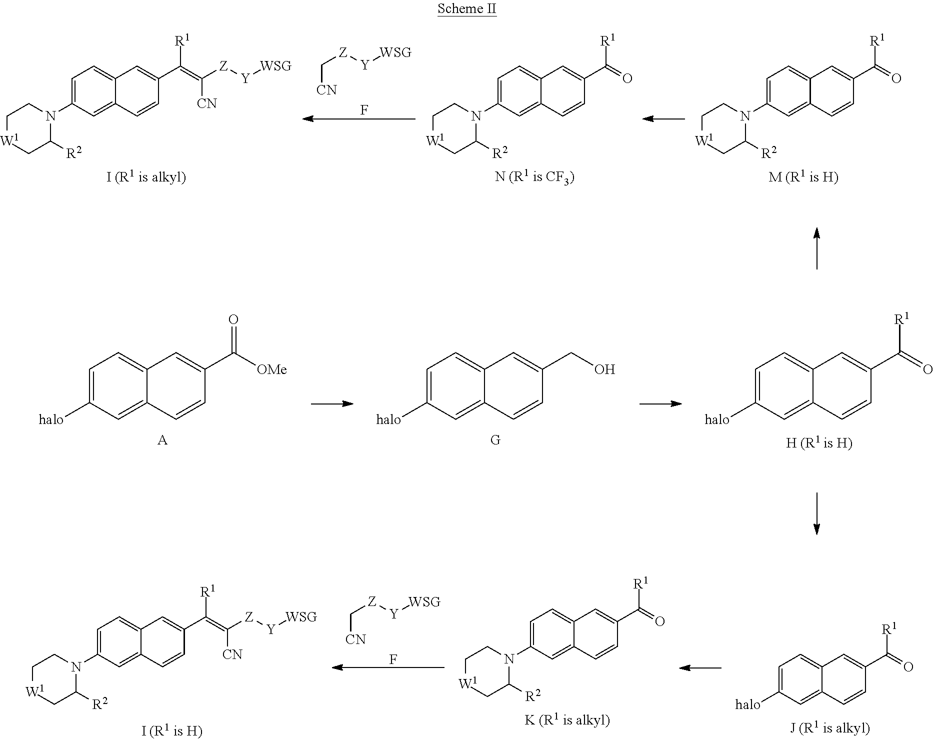

Halo substituted naphthalene, compound A, is reduced under suitable reaction conditions such as a metal hydride agent in a solvent such as THF to provide compound G. Oxidation of compound G under suitable reaction conditions such as PCC in a solvent such as methylene dichloride provides compound H (wherein R¹ is H). Coupling of compound H with piperidine compound B under suitable reaction conditions such as palladium catalyst and a base such as Cs₂CO₃ in a solvent such as toluene provides compound M, wherein R¹ is H. Nucleophilic reaction of compound H (wherein R¹ is H) with a suitable trifluoromethyl compound such as (CF₃)SiMe₃ in a solvent such as THF under suitable reaction conditions provides compound N (wherein R¹ is CF₃). Coupling of compound N with compound F under suitable reaction conditions in presence of a base such as piperidine in a suitable solvent such as THF provides compounds of formula I or II, wherein R¹ is CF₃. Alternatively, Grignard reaction of compound H (wherein R¹ is H) with a suitable alkyl magnesium halide in a solvent such as THE under suitable reaction conditions provides compound J (wherein R¹ is alkyl). Coupling of compound J with compound B under suitable reaction conditions provides compound K. Coupling of compound K with compound F under suitable reaction conditions such as palladium catalyst and a base such as Cs₂CO₃ in a solvent such as toluene provides compounds of formula I or II, wherein R¹ is alkyl.

The prodrugs of compounds of formula I or II can be prepared by the methods disclosed in WO 2020/093008. See for example, Scheme III below.

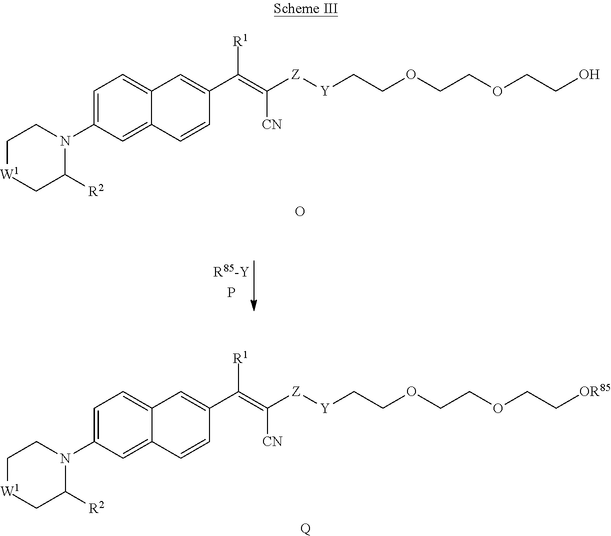

In Scheme III, an exemplary compound of formula I or II, compound O, is reacted with compound P under suitable reaction conditions to provide compound Q, where R⁸⁵ is a prodrug moiety, such as a phosphate prodrug, as described herein. In some cases, compound O is deprotonated using a base and then contacted with compound P.

### Detectable Target Proteins

A compound described herein may be useful in detecting or treating a neurological disease or disorder. In this regard, a compound described herein may be a prodrug.

Many neurological diseases, including neurodegenerative diseases and injury-related disorders may be detected by the compounds and methods described herein. The neurological disease or disorder may be characterized by certain peptides, protein, or accumulated mass of protein, described herein as detectable proteins. The detectable protein, or the accumulated mass thereof, may comprise, for example, amyloid beta protein, alpha-synuclein (aSyn), TAR DNA-binding protein 43 (TDP43), or phosphorylated tau protein. The amyloid beta protein, alpha-synuclein (aSyn), TAR DNA-binding protein 43 (TDP43), or phosphorylated tau protein may be detected by contacting with a compound, as described herein. Generally, the compounds and methods described herein are useful for detection of amyloid beta protein, alpha-synuclein (aSyn), TAR DNA-binding protein 43 (TDP43), or phosphorylated tau protein, or accumulated mass thereof, in a tissue or a sample of the patient. Such presence of amyloid beta protein or phosphorylated tau protein can be detected with compounds that bind to the amyloid beta protein or phosphorylated tau protein, which binding can then be detected.

Amyloid beta-protein (Aβ) is a polypeptide generally containing about 40 amino acid residues, e.g., about 36-43, about 39-43, or about 40-42 amino acid residues. Isoforms include Aβ(1-40) and Aβ(1-42). In some embodiments, the Aβ is Aβ(1-42). Aβ is believed to be produced by enzymatic cleavage of a larger precursor protein, beta-amyloid precursor protein (APP), which is encoded by a gene on human chromosome 21. APP isoforms include NP_000475.1, NP_001129488.1, NP_001129601.1, NP_001129602.1, NP_001129603.1, NP_001191230.1, NP_001191231.1, NP_001191232.1, NP_958816.1, and NP_958817.1. Aβ is believed to be generated by action of the enzymes β and γ secretases on APP. Aβ has been found in deposits, e.g., plaques, in the brains of individuals having Alzheimer's disease. It is thought that Aβ is involved in the pathogenesis of neurological diseases. As is also believed to be toxic to nerve cells.

The protein that is detected by a compound of the disclosure include amyloid beta peptide (Ap), prion peptide (PrP), alpha-synuclein, IAPP (amylin), huntingtin, calcitonin (ACal), atrial natriuretic factor (AANF), apolipoprotein A1 (ApoA1), serum amyloid A (SAA), medin (AMed), prolactin (APro), transthyretin (ATTR), lysozyme (ALys), beta 2 microglobulin (Aβ2M), gelsolin (AGel), keratoepithelin (Aker), cystatin (ACys), immunoglobulin light chain AL (AL), S-IBM or superoxide dismutase. In some embodiments, the amyloid peptide detected is Aβ peptide, prion peptide, alpha-synuclein, or superoxide dismutase.

“Microtubule associated protein tau,” “MAPT,” “tau protein,” or “tau” are a family of proteins which stabilize microtubules during assembly and disassembly, and are classified as microtubule-associated proteins (MAPs). Tau isoform sequences include NP_001116538.2, NP_001116539.1, NP_001190180.1, NP_001190181.1, NP_005901.2, NP_058518.1, NP_058519.3, and NP_058525.1. Tau proteins are important in the stabilization and assembly of microtubules, and in turn, affect the intraneuronal transport of cargos. Tau may also be involved in signaling pathways by interacting with actin via acidic N-terminals, projecting from microtubules for neurite outgrowth and stabilization during brain development. A tau protein as provided herein may comprise any isoform, or any combination of isoforms. MAPT transcripts are differentially expressed in the nervous system, depending on stage of neuronal maturation and neuron type. MAPT gene mutations have been associated with several neurological disorders such as Alzheimer's disease, Pick's disease, frontotemporal dementia, cortico-basal degeneration and progressive supranuclear palsy. The tau protein may or may not include post-translational modifications. The tau protein family is characterized by an N-terminal segment shared by all members, sequences of ~50 amino acids inserted in the N-terminal segment, which are developmentally regulated in the brain, a characteristic tandem repeat region consisting of 3 or 4 tandem repeats of 31-32 amino acids, and a C-terminal tail.

The human tau gene is located on the long arm of chromosome 17 at position 17q21. The gene is believed to contain 16 exons, with exon 21 as a part of the promoter. The tau primary transcript contains 13 exons, and exons 4A, 6 and 8 are not transcribed in human. Exons 21 and 14 are transcribed but not translated. Exons 1, 4, 5, 7, 9, 11, 12, 13 are constitutive, and exons 2, 3, and 10 are alternatively spliced, giving rise to six different mRNAs, translated in six different tau isoforms. These isoforms differ by the absence or presence of one or two 29 amino acid repeat (0N, 1N, or 2N) encoded by exon 2 and 3 in the amino-terminal part, in combination with either three microtubule binding repeats (R1, R3 and R4) or four (R1-R4) repeat-regions in the carboxy-terminal part. The fourth microtubule-binding domain is encoded by exon 10. Six tau protein isoforms are known to exist in human brain tissue: (2+3+10+) isoform (having 441-amino acids), (2+3+10−) isoform (having 410-amino acids), (2+3-10+) isoform (having 412-amino acids), (2+3-10−) isoform (having 381-amino acids), (2-3-10+) isoform (having 383-amino acids), and (2-3-10−) isoform (having 352-amino acids). The tau may be a mutant tau. The mutation may be a FTDP-17 mutation. Examples of mutations include G272V, N279K, N296, P201L, P301S, G303V, S305N, L315R, S320F, P332L, V337M, E342V, S352L, K369I, G389R, R5H, R5L, K257T, I260V, L266V, G272V, delK280, N296H, N296N, delN296, P301L, P301S, K317M, G335V, Q336R, R406W and R427M.

“Phosphorylated tau protein” or “phosphorylated tau” is a tau protein having at least one amino acid residue modified by a phosphate group. Tau is believed to include as many as 85 amino acid residues compatible with phosphorylation. Generally, the phosphate group is a post-translational modification and may be bonded at a side chain of an amino acid residue. The phosphorylated amino acid residue may be, for example, a serine (S), threonine (T), or tyrosine (Y) residue, or a combination thereof. A phosphorylated tau protein may include 1, 2, 3, 4, 5, 6, 7, 8, 9, 10, 11, 12, 13, 14, 15, 16, 17, 18, 19, 20, at least 20, at least 25, at least 30, at least 40, or at least 50 moles of phosphate per mole of protein. A phosphorylated tau protein may include at least 3 moles of phosphate per mole of protein. A phosphorylated tau protein may include one or more phosphorylated amino acid residues selected from Thr39, Ser46Pro, Thr50Pro, Thr69Pro, Thr153Pro, Thr175Pro, Thr181Pro, Ser198, Ser199, Ser202Pro, Thr205Pro, Ser208, Ser210, Thr212Pro, Ser214, Thr217Pro, Thr231Pro, Ser235Pro, Ser237, Ser241, Ser262, Ser285, Ser305, Ser324, Ser352, Ser356, Ser396Pro, Ser400, Thr403, Ser404Pro, Ser409, Ser412, Ser413, Ser416, and Ser422Pro. The phosphorylated tau protein may include phosphorylated Ser422. A phosphorylated tau protein as provided herein may be aggregated or may be unaggregated. A phosphorylated tau protein as provided herein may be soluble. A tau protein, or a phosphorylated tau protein, may be a three-repeat tau, a four-repeat tau, or a combination thereof. In some embodiments, a tau protein, or a phosphorylated tau protein, may comprise a mixture of three-repeat tau and four-repeat tau in which four-repeat tau is more prevalent. In some embodiments, a tau protein, or a phosphorylated tau protein, may comprise a mixture of three-repeat tau and four-repeat tau in which three-repeat tau is more prevalent. The phosphorylated tau protein may be fibrillary, for example as a neurofibrillary tangle (NFT). The NFT may be found in the somatodendritic compartments of neurons.

Alpha-synuclein (aSyn) is a neuronal protein found in abundance in the brain, mainly in the axon terminals of presynaptic neurons. Synucleopathies are a type of neurodegenerative diseases associated with the abnormal accumulation of aggregates of aSyn, and includes Parkinson's disease (PD), Lewy body dementia (LBD), multiple system atrophy (MSA), and the like.

TAR DNA-binding protein 43 (TDP43) is a 43 kDa protein that harbors most of the mutations associated with familial amyotrophic lateral sclerosis (ALS). Hyper-phosphorylated, ubiquinated and cleaved form of TDP-43 is known as pathologic TDP43 and is a major disease protein associated with conditions such as amyotrophic lateral sclerosis (ALS). ALS is a terminal neurodegenerative disease that results in progressive loss of motor neurons that control voluntary muscles. Misfolded TDP-43 has also been linked to limbic-predominant age-related TDP-43 encephalopathy (LATE), a form of dementia.

“Contacting” is used in accordance with its plain ordinary meaning and refers to the process of allowing at least two distinct species (e.g. chemical compounds including biomolecules, or cells) to become sufficiently proximal to interact. The term “contacting” may include allowing two molecular species to react or physically touch, wherein the two species may be, for example, a compound as described herein, a biomolecule, a protein or an enzyme. In some embodiments contacting includes allowing a compound described herein to interact with a protein (e.g., Aβ protein or a phosphorylated tau protein) or enzyme.

In accordance with some embodiments of the present disclosure, therefore, provided is a method for determining whether a patient has a neurological disease or disorder. The method entails detecting the presence of an amyloid beta-protein or a phosphorylated tau protein, or an accumulated mass thereof, in a tissue or a sample of the patient by contacting the tissue or sample of the patient with a compound described herein. The contacting may be in vivo or ex vivo. The contacting may be by administration, for example, topical or intravenous administration, of the compound to a patient.

In another embodiment, provided is a method for preparing a patient for diagnosis of a neurological disease or disorder, which method comprises administering to the patient a compound described herein, and binding to an amyloid beta-protein or a phosphorylated tau protein, or an accumulated mass thereof. The compound may be administered intravenously. Once the compound is administered to the patient, its binding, and/or the binding of a parent compound, to the amyloid beta-protein or phosphorylated tau protein, or accumulated mass thereof, may be detected with any method, including those methods described herein. In some embodiments, the binding indicates a likelihood that the patient has a neurological disease or disorder.

### Neurological Diseases and Disorders and Treatment Thereof

Proteinopathies are a family of diseases caused by misfolded proteins. A hallmark of neurodegenerative proteinopathies is the formation of misfolded protein aggregates that cause cellular toxicity and contribute to cellular proteostatic collapse. Misfolded or aggregated proteins may be detected by contacting the same with a compound as described herein, wherein the contacting, upon activation by a light, causes emission of detectable signal. Generally, the compounds, compositions, and methods described herein are useful for detection of misfolded or aggregated proteins, such as amyloid beta (Aβ, including both isoforms Aβ40 and Aβ42), phosphorylated tau, alpha-synuclein (aSyn), and TAR DNA-binding protein 43 (TDP43).

In some embodiments, the disclosure provides a method of determining the presence or absence of a neurological disease or disorder in a patient. In some embodiments, the method comprises administering to the patient an effective amount of a compound described herein, or a pharmaceutical composition thereof. The compound may be a compound of Formula I or II. In some embodiments, a method for determining whether a patient has a neurological disease or disorder is provided, comprising administering to the patient a compound described herein, or a pharmaceutical composition described herein. In some embodiments of the method, the compound is administered intravenously. In some embodiments of the method, the compound is administered to the eye of the patient. In some embodiments, the neurological disease or disorder is a disease or disorder characterized by protein aggregation or protein misfolding.

Also provided herein are methods for determining whether a patient has a neurological disease or disorder, comprising detecting the presence of an amyloid beta protein or a phosphorylated tau protein, or an accumulated mass thereof, in a tissue or a sample of the patient wherein the detecting comprises contacting the tissue or the sample with a compound described herein. The compound may be a compound of Formula I or II. The contacting may be in vivo. The tissue may be an eye tissue. The sample may be a urine sample.

In some embodiments, the neurological disease or disorder is selected from an age-related disease or disorder, a genetic disease or disorder, an injury-related disease or disorder, and a psychiatric disease or disorder. In some embodiments, the age-related disease or disorder is selected from Parkinson's disease, vascular dementia, Alzheimer's disease, cerebral amyloid angiopathy, frontotemporal dementia, limbic-predominant age-related TDP-43 encephalopathy, Lewy body dementia, multiple system atrophy, progressive supranuclear palsy, age-related macular degeneration, and Amyotrophic lateral sclerosis, the genetic disease or disorder is Down syndrome, the injury-related disease or disorder is selected from traumatic brain injury and chronic traumatic encephalopathy, and the psychiatric disease or disorder is selected from schizophrenia and depression. The neurological disease or disorder may be a tauopathy. In some embodiments, the neurological disease or disorder is Alzheimer's disease or traumatic brain injury (TBI).

The neurological disease or disorder may be a tauopathy. Tauopathies are a class of neurological diseases associated with the pathological aggregation of tau protein in neurofibrillary or gliofibrillary tangles in the human brain. Tangles may be formed by hyperphosphorylation of tau, causing the tau protein to dissociate from microtubules and form aggregates in an insoluble form. The aggregations of hyperphosphorylated tau protein may also be referred to as paired helical filaments. The precise mechanism of tangle formation is not completely understood, and it is still controversial as to whether tangles are a primary causative factor in the disease or play a more peripheral role. Tauopathy has been found in many neurological disorders, such as posttraumatic degeneration, infections, metabolic diseases, and motor neuron degeneration. The spatial distribution, temporal appearance, and structural changes of tau proteins manifest differently among various neurological diseases. AD patients have twisted, hyperphosphorylated, and single nonperiodical tau filaments, whereas patients having progressive supernuclear palsy and frontotemporal dementia (FTD) tend to have only straight tau filaments. Tauopathies are often overlapped with synucleinopathies, possibly due to interaction between the synuclein and tau proteins. Non-Alzheimer's tauopathies are sometimes grouped together as “Pick's complex” due to their association with frontotemporal dementia, or frontotemporal lobar degeneration. A marker of tau hyperphosphorylation is tau pS422. Chronic traumatic encephalopathy (CTE) is associated with repetitive mild traumatic brain injury (mTBI), and bears many similarities with tauopathies, including hyperphosphorylation and aggregation of tau, for example, as neurofibrillary tangles (NFTs).

In some embodiments, the neurological disease or disorder is selected from primary age-related tauopathy (PART), neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), Lytico-bodig disease (Parkinson-dementia complex of Guam), ganglioglioma, gangliocytoma, meningioangiomatosis, postencephalitic parkinsonism, subacute sclerosing panencephalitis (SSPE), lead encephalopathy, tuberous sclerosis, Pantothenate kinase-associated neurodegeneration, and lipofuscinosis.

The neurological disease or disorder may be a neurodegenerative disease or disorder. In some embodiments, the neurological disease or disorder is Alzheimer's disease (AD). AD may be classified as a secondary tauopathy. Alzheimer's disease is characterized by symptoms of memory loss in the early stages of the disease. Neurofibrillary tangles were an early descriptor of AD. When tau becomes hyperphosphorylated, the protein dissociates from the microtubules in axons. Then, tau may become misfolded and begin to aggregate, which may form neurofibrillary tangles (NFT). Microtubules also destabilize when tau is dissociated, and the combination of the neurofibrillary tangles and destabilized microtubules result in disruption of processes such as axonal transport and neural communication. The degree of NFT involvement in AD is defined by Braak stages. Braak stages I and II are used when NFT involvement is confined mainly to the transentorhinal region of the brain; stages III and IV when limbic regions such as the hippocampus become involved; stages V and VI when extensive neocortical involvement is indicated. AD is also classified as an amyloidosis because of the presence of senile plaques. Additionally, certain Apoε4 carriers are at greater risk of developing AD. APOε4 is believed to be less efficient than other isoforms at clearing Aε, and thus may be correlated with greater amyloid burden, tau phosphorylation, synaptotoxicity, and reduced synaptic density. Having experienced a traumatic brain injury (TBI) is another risk factor for developing AD, and studies indicate that those who experience a TBI have a significantly increased risk of AD.

As AD advances, symptoms include confusion, long-term memory loss, paraphasia, loss of vocabulary, aggression, irritability and/or mood swings. In more advanced stages of the disease, there is loss of bodily functions. Patients with Alzheimer's Disease (AD) demonstrate many characteristic neuropathies such as increased oxidative stress, mitochondrial dysfunction, synaptic dysfunction, disruption of calcium homeostasis, deposition of senile plaques and neurofibrillary tangles, and atrophy of the brain. AD related disorders include senile dementia of AD type (SDAT), frontotemporal dementia (FTD), vascular dementia, mild cognitive impairment (MCI) and age-associated memory impairment (AAMI). In some embodiments, determining whether a patient has Alzheimer's disease, comprising detecting the presence of a phosphorylated tau protein in a tissue or a sample of the patient, wherein the detecting comprises contacting the phosphorylated tau protein with a compound described herein.

In some embodiments, the neurological disease or disorder disease is frontotemporal lobar degeneration (FTLD) (e.g., FTLD-tau, FTLD-TDP, or FTLD-FUS). In some embodiments, the neurological disease or disorder is frontotemporal lobe dementia. In some embodiments, the neurological disease or disorder includes memory loss. In some embodiments, the neurological disease or disorder is age-related memory loss. In some embodiments, the neurological disease or disorder is FTLD-TDP Type A. In some embodiments, the neurological disease or disorder is FTLD-TDP Type B. In some embodiments, the neurological disease or disorder is FTLD-TDP Type C. In some embodiments, the neurological disease or disorder is FTLD-TDP Type D.

In some embodiments, the neurological disease or disorder is Parkinson's disease. In some embodiments, the neurological disease or disorder is Parkinson's dementia. In some embodiments, the neurological disease or disorder is related to (e.g. characterized by) an accumulated mass of amyloid plaques. In some embodiments, a patient having a neurological disease or disorder has suffered a traumatic brain injury before, during, or after the onset of the neurological disease or disorder. In some embodiments, the neurological disease or disorder includes a neuronal impairment. A neuronal impairment may include atrophy or other decrease in the effective functioning of the neuron. For example, it is known that Alzheimer's disease presents with neuronal impairment, especially in cortical neurons, e.g., hippocampal neurons and neurons in proximity to the hippocampus.

In some embodiments, the neurological disease or disorder is traumatic axonal injury (TAI), traumatic brain disorder (TBD), dementia (e.g., general dementia), frontotemporal dementia with parkinsonism linked to chromosome 17 (FTDP-17), primary age-related tauopathy (PART), neurofibrillary tangle-predominant senile dementia, progressive supranuclear palsy (PSP), corticobasal degeneration, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, Pick's disease, corticobasal degeneration, argyrophilic grain disease (AGD), or corticobasal degeneration.

The neurological disease or disorder may be an injury-related condition such as traumatic brain injury (TBI) or chronic traumatic encephalopathy (CTE). TBI is a chronic disease from damage to the brain caused by an external force, such as a bump, blow, jolt, rapid acceleration or deceleration, or penetration by a projectile. Injury leading to TBI may produce diminished or altered states of consciousness, resulting in temporary or permanent impairment in cognition, sensorimotor, and psychosocial function. CTE is a progressive degenerative disease found in people who have suffered repetitive brain trauma, including hits to the head that did not result in TBI symptoms. Physical aspects of CTE include shrinking of the brain, atrophy of the frontal and temporal lobes, enlargement of the ventricles, atrophy of the hippocampus, thalamus, brainstem and cerebellum. Individuals with CTE may have symptoms of dementia, memory loss, aggression, confusion, depression and suicidal ideations that may occur many years after the injuries.

The neurological disease or disorder may be of the eye, for example, glaucoma, ocular hypertension, macular degeneration, diabetic retinopathy, age-related macular degeneration (AMD) or retinitis pigmentosa.

In some embodiments of the method, the neurological disease or disorder is a prion disease. Prion diseases are fatal, transmissible, neurological disorders, the most prominent of which is Creutzfeldt-Jakob disease or CJD. CJD is sometimes called the “great mimicker” because it causes symptoms that occur in many other neurological diseases. Common CJD symptoms include different neurological and psychiatric indications, including behavioral changes, confusion, cognitive dysfunction, movement problems, and visual disturbances. CJD is difficult to diagnose, with current practices relying on cerebral spinal fluid tests. Interestingly, recent reports have indicated prion proteins in post-mortem retinal tissue of CJD patients.

Accordingly, provided is a method for detecting prion deposition in a patient. In certain embodiments, the detecting is performed in the retina. In certain embodiments, provided is a method for detecting prion deposition in the retina of a patient, wherein the patient may or may not exhibit clinical manifestations of a disease or disorder related thereto, such as Creutzfeldt-Jakob disease (e.g., behavioral changes, confusion, cognitive dysfunction, movement problems, visual disturbances, kyphosis, ataxia, tip toe walking, etc.).

Cerebral amyloid angiopathy (CAA) is a disease of aging characterized by amyloid deposition within cerebral blood vessel walls. These deposits develop in cortical and leptomeningeal arteries, leading to an increased risk of spontaneous intracerebral hemorrhage, ischemic lesions, and progressive dementia in the elderly population. The diagnosis of CAA is often missed by physicians as the presenting symptoms are those similar to a transient ischemic attack or “mini-stroke.” Diagnosis can be further complicated as CAA is found in up to 90% of Alzheimer's disease patients. Several amyloid forming peptides can lead to CAA; among them, amyloid beta (Aβ) is by far the most prevalent form. AR deposition has been detected within the walls of arterioles, capillaries, and arteries of severe CAA human brain tissue. It has become established that the Aβ42 isoform is preferentially present in the parenchymal plaques of Alzheimer's patients while the Aβ40 isoform is denser within brain vascular wall depositions in CAA. In some embodiments of the method, the neurological disease or disorder is cerebral amyloid angiopathy (CAA). Cerebral amyloid angiopathy (CAA) is a disease of aging characterized by amyloid deposition within cerebral blood vessel walls.

Accordingly, in certain embodiments, provided is a method for detecting Aβ40 in a patient. In certain embodiments, the detecting is performed in the retina. In certain embodiments, the detecting differentiates between the Aβ isoforms associated with CAA (Aβ40) and Alzheimer's disease (Aβ42).

In some embodiments, provided is a method for detecting alpha-synuclein (aSyn) in a patient. Alpha-synuclein has been associated with various neurological conditions such as Parkinson's disease and Lewy body dementia (LBD).

In some embodiments, provided is a method for detecting TAR DNA-binding protein 43 (TDP43) in a patient. Hyper-phosphorylated, ubiquinated and cleaved form of TDP-43 is known as pathologic TDP43 and is a major disease protein associated with conditions such as amyotrophic lateral sclerosis (ALS).

In some embodiments, the neurological disease or disorder is accompanied by protein that produces amyloid like morphology and disease or conditions associated with the formation of abnormal protein structures, protein aggregation, or protein misfolding. Non limiting examples of amyloid-based diseases include Alzheimer's disease, Parkinson's disease, Huntington's disease, Down Syndrome, and spongiform encephalopathies such as, for example, bovine spongiform encephalopathy (mad cow disease), kura, Creutzfeldt-Jakob disease, and fatal familial insomnia. In some cases, other amyloid based diseases that are detected, treated or prevented by the methods of the disclosure include reactive systemic amyloidosis, senile systemic amyloidosis (SAA), familial amyloid polyneuropathy (FAP), familial amyloid cardiomyopathy (FAC), prion disease, coronary heart disease, atherosclerosis, cerebral hemorrhage, AL amyloidosis, type 2 diabetes, diseases or conditions characterized by a loss of cognitive memory capacity such as, for example, mild cognitive impairment (MCI), Lewy body dementia (LBD), hereditary cerebral hemorrhage with amyloidosis (Dutch type) and the Guam Parkinson-Dementia complex. Other diseases which are based on or associated with amyloid-like proteins are progressive supranuclear palsy, multiple sclerosis, HIV-related dementia, ALS (amyotrophic lateral sclerosis), inclusion-body myositis (IBM), Adult Onset Diabetes; endocrine tumors, and other diseases, including amyloid-associated ocular diseases that target different tissues of the eye, such as the visual cortex, including cortical visual deficits; the anterior chamber and the optic nerve, including glaucoma; the lens, including cataract due to beta-amyloid deposition; the vitreous, including ocular amyloidosis; the retina, including primary retinal degenerations and macular degeneration, in particular age-related macular degeneration; the optic nerve, including optic nerve drusen, optic neuropathy and optic neuritis; and the cornea, including lattice dystrophy.

Additional examples of a neurological disease or disorder include Alexander's disease, Alper's disease, depression, perinatal asphyxia, Parkinson's disease dementia (“PD dementia”), amyotrophic lateral sclerosis, ataxia telangiectasia, Batten disease (also known as Spielmeyer-Vogt-Sjogren-Batten disease), spongiform encephalopathy (e.g., bovine spongiform encephalopathy (mad cow disease), Kuru, Creutzfeldt-Jakob disease, fatal familial insomnia, Canavan disease, Cockayne syndrome, corticobasal degeneration, fragile X syndrome, frontotemporal dementia, Gerstmann-Straussler-Scheinker syndrome, Huntington's disease, HIV-associated dementia, Kennedy's disease, Krabbe's disease, Lewy body dementia, Machado-Joseph disease (Spinocerebellar ataxia type 3), multiple sclerosis, multiple system atrophy, narcolepsy, neuroborreliosis, Pelizaeus-Merzbacher Disease, primary lateral sclerosis, prion diseases, Refsum's disease, Sandhoff disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, Tabes dorsalis, drug-induced Parkinsonism, progressive supranuclear palsy, corticobasal degeneration, multiple system atrophy, idiopathic Parkinson's disease, autosomal dominant Parkinson's disease, familial, type 1 (PARK1), Parkinson's disease 3, autosomal dominant Lewy body (PARK3), Parkinson's disease 4, autosomal dominant Lewy body (PARK4), Parkinson's disease 5 (PARK5), Parkinson's disease 6, autosomal recessive early-onset (PARK6), Parkinson's disease 2, autosomal recessive juvenile (PARK2), Parkinson's disease 7, autosomal recessive early-onset (PARK7), Parkinson's disease 8 (PARK8), Parkinson's disease 9 (PARK9), Parkinson's disease 10 (PARK10), Parkinson's disease 11 (PARK11), Parkinson's disease 12 (PARK12), Parkinson's disease 13 (PARK13), and mitochondrial Parkinson's disease.

Upon determination of a neurological disease or disorder in a patient, certain procedures can be provided to treat or ameliorate the symptoms of the neurological disease or disorder, or to slow or halt the progression thereof. Once a neurological disease or disorder is diagnosed, the progression of the disease or disorder may also be monitored by the methods described herein. Once diagnosed, the treating physician may also suggest additional treatments as known to practitioners, including those described herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) ameliorating, slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a patient (including a human) who is at risk or has a family history of the disease or condition.

“Patient” refers to an animal, such as a mammal (including a human), that has been or will be the object of diagnosis, treatment, observation or experiment. The methods described herein may be useful in human and/or veterinary applications. In some embodiments, the patient is a mammal. In one embodiment, the patient is a human.

The term “effective amount” of a compound described herein or a pharmaceutically acceptable salt, tautomer, stereoisomer, mixture of stereoisomers, prodrug, or deuterated analog thereof means an amount sufficient to detect an amyloid beta protein or phosphorylated tau protein, or an accumulated mass thereof, when administered to a patient or a sample of the patient, or to provide a therapeutic benefit such as amelioration of symptoms or slowing of disease progression. For example, an effective amount may be an amount sufficient to decrease a symptom of a disease or condition of a neurological disease or disorder. The effective amount may vary depending on the patient, and disease or condition being treated, the weight and age of the patient, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal dosing of administration of a compound of the present disclosure for a given indication, cell type, individual, and other parameters. Information gleaned from such use may be used for experimental purposes or in the clinic to set protocols for in vivo use. Other ex vivo uses for which the compounds and compositions described herein may be suited are described below or will become apparent to those skilled in the art. The selected compounds may be further characterized to examine the safety or tolerance dosage in human or non-human patients. Such properties may be examined using commonly known methods to those skilled in the art.

### Detection of Target Protein

The neurological diseases or disorders described herein are characterized by certain peptides, protein, or protein aggregates, the detection of which is useful for diagnosis. The peptides, protein, or the accumulated mass thereof, are collectively referred to as detectable target proteins and may comprise, for example, amyloid beta protein (Aβ), alpha-synuclein (aSyn), TAR DNA-binding protein 43 (TDP43), or phosphorylated tau protein.

Provided herein are methods for diagnosis of a neurological disease or disorder in a patient, comprising administering to a tissue of the patient a compound described herein. The compound may be a compound of Formula I or IL. The method may comprise detecting a binding of the compound, and/or the binding of a parent compound, to a detectable target protein, for example, an amyloid beta protein, or a phosphorylated tau protein, or an accumulated mass thereof. The administration may be intravenous administration. The method may comprise detecting a binding of the compound to a detectable target protein. In some embodiments, the method further comprises activation by a light and emission of a detectable signal. In some embodiments, the method includes comparing the signal to a control value, wherein an increase in the signal compared to the control value indicates a presence of detectable target protein, where the control value is the signal in the absence of a detectable target protein. In some embodiments of the method, the detectable signal is a fluorescent or infrared signal. In some embodiments, the light is a laser.

In some embodiments, the disclosure provides a method of detecting a detectable target protein, for example, an amyloid beta protein, or a phosphorylated tau protein, or an accumulated mass thereof. The method comprises contacting a compound described herein with a tissue or a sample potentially comprising the detectable target protein, for example, an amyloid beta protein, or a phosphorylated tau protein, or an accumulated mass thereof, wherein the compound binds with the detectable target protein. In some embodiments, provided herein are methods of detecting the presence or absence of binding of a compound described herein, or a parent compound thereof, with a detectable target protein, comprising administering to a patient a compound, or a pharmaceutically acceptable salt thereof, as described herein. In some embodiments, provided herein are methods for monitoring the response of a patient having a disease or condition characterized by the presence of a detectable target protein to a treatment, comprising binding to the detectable target protein following the treatment an effective amount of a compound described herein, or a pharmaceutically acceptable salt thereof, and detecting a signal created in response to the binding, wherein a decrease of signal as compared to before the treatment indicates that the patient is responsive to the treatment. In some embodiments, the detectable target protein is an amyloid or amyloid like protein, for example, Aβ peptide, prion peptide, alpha-synuclein, or superoxide dismutase. In some embodiments, the amyloid or amyloid like protein is beta amyloid (1-42) (Aβ(1-42)). In some embodiments, the detectable target protein is a phosphorylated tau protein. In some embodiments, the phosphorylated tau protein is a three repeat tau or a four repeat tau.

In some embodiments, detection is performed within about 1 sec, about 5 sec, about 1 min, about 10 min, about 30 min or about 60 min of the contacting of the compound with the detectable target protein, or administration of the compound. In some embodiments, detection is performed within about 1-5 minutes of the contacting of the compound, or administration of the compound.

In situ detection of binding of detectable target protein, for example, an amyloid beta protein, or a phosphorylated tau protein, or an accumulated mass thereof, with a compound described herein, can be facilitated with an imaging device, which is preferably handheld or portable. The imaging device can include a lens and an image sensor, and optionally a laser light source. When the light source emits laser light to the tissue, for example, the retina, if detectable target protein is accumulated there and has bound to a compound described herein, the target protein can be readily detected and quantitated by the lens and image sensor that collects and senses a fluorescent signal. The imaging device may be any device capable of detecting light, for example, a camera. The imaging device may comprise a confocal lens. The imaging device may be a retinal imaging device. The imaging device may comprise a fundus camera. The detection may comprise confocal laser scanning microscopy. The detection may be non-mydriatic. For an overview of retinal imaging techniques, see, e.g., M. D. Abràmoff et al, Retinal Imaging and Image Analysis (2010), IEEE Rev Biomed Eng. 3:169-208.

The amyloid beta protein or phosphorylated tau protein may accumulate in an eye of the patient. In some embodiments, the contacting, upon activation by a light, causes emission of detectable signal. The signal may be a fluorescent or infrared signal.

Provided herein is a method for treating a neurological disease or disorder in a patient, comprising administering to the patient a compound described herein. The compound may be a compound of Formula I or II.

### Administration and Pharmaceutical Compositions

Also provided are pharmaceutical compositions of compounds described herein for administration to a patient. The compound may be a compound of Formula I or II. The compound may be administered in either single or multiple doses. The compound may be administered by various methods including, for example, rectal, buccal, intranasal and transdermal routes. In certain embodiments, the compound may be administered by intra-arterial injection, intravenously, intraperitoneally, parenterally, intramuscularly, subcutaneously, orally, topically, or as an inhalant. In some embodiments, a compound described herein is administered intravenously. The intravenous administration can be bolus administration or continuous injection. Additional modes of injection include intraarterial, intracardiac, intrathecal, intraosseous, intraarticular, intrasynovial, intracutaneous, subcutaneous, intramuscular, and intradermal, intracranial, intralesional, and intratumoral.

In some embodiments, a compound described herein is administered to the eye. In some embodiments, the compounds are administered topically to the eye. In some embodiments, the administration is parenteral, for example, by injection. In some embodiments, the administration is oral.

The compound may be effective over a wide dosage range. In some embodiments, the dose is from 0.01 to 1000 mg, from 0.5 to 100 mg, from 1 to 50 mg per day, or from 5 to 40 mg. Exemplary dosages include 10, 20, 30, 50, 75, 100, 200, 300, 400, 500, 600, 700, 800, 900, and 1000 mg. In some embodiments the effective amount of the compound corresponds to about 50 to 500 mg. An effective amount may vary between individual patients. The exact dosage will depend upon the route of administration, the form in which the compound is administered, the patient to be treated, the body weight or surface area of the patient to be treated, and the preference and experience of the attending physician.

In some embodiments the effective amount of the compound is about 0.01-1000 mg per dose. In some embodiments, the effective amount of compound is 50-500 mg per dose. In some embodiments the effective amount is about 0.01-100 mg, 0.01-200 mg, 0.01-300 mg, 0.01-400 mg, 0.01-500 mg, 0.01-600 mg, 0.01-700 mg, 0.01-800 mg, 0.01-900 mg, 0.01-1000 mg, 0.1-100 mg, 0.1-200 mg, 0.1-300 mg, 0.1-400, 0.1-500 mg, 0.1-600 mg, 0.1-700 mg, 0.1-800 mg, 0.1-900 mg, 0.1-1000 mg, 1-100 mg, 1-200 mg, 1-300 mg, 1-400 mg, 1-500 mg, 1-600 mg, 1-700 mg, 1-800 mg, 1-900 mg, 100-200 mg, 100-300 mg, 100-400 mg, 100-500 mg, 100-600 mg, 100-700 mg, 100-800 mg, 100-900 mg, 100-1000 mg, 200-300 mg, 200-400 mg, 200-500 mg, 200-600 mg, 200-700 mg, 200-800 mg, 200-900 mg, 200-1000 mg, 300-400 mg, 300-500 mg, 300-600 mg, 300-700 mg, 300-800 mg, 300-900 mg, 300-1000 mg, 400-500 mg, 400-600 mg, 400-700 mg, 400-800 mg, 400-900 mg, 400-1000 mg, 500-600 mg, 500-700 mg, 500-800 mg, 500-900 mg, 500-1000 mg, 600-700 mg, 600-800 mg, 600-900 mg, 600-1000 mg, 700-800 mg, 700-900 mg, 700-1000 mg, 800-900 mg, 800-1000 mg or about 900-1000 mg per dose. In some embodiments, the effective amount is about 50-100 mg, 50-400 mg, 50-500 mg, 100-200 mg, 100-300 mg, 100-400 mg, 100-500 mg, 200-300 mg, 200-400 mg, 200-500, 300-400 mg, 300-500 mg, or 400-500 mg per dose.

In some embodiments, the compound is administered in a single dose. In some embodiments, the compound is administered in multiple doses.

In some embodiments, the compound is administered in a pharmaceutical composition comprising a liquid carrier, for example, for intravenous administration. In some embodiments, the volume of pharmaceutical composition is from about 10 μL to about 1000 mL. For example the volume may be about 10 μL, 50 μL, 100 μL, 300 μL, 500 μL, 1 mL, 10 mL, 50 mL, 100 mL, 200 mL, 300 mL, 400 mL, 500 mL, 600 mL, 700 mL, 800 mL, 900 mL, or 1000 mL.

In some embodiments, the compound is administered as drops. In some embodiments, the size of the drop administered is in the range of about 10-100 μL, about 20-50 μL, or about 50-80 μL. In some embodiments, the drops are administered several drops per administration, for example 1-3 drops per time, 3-10 drops per time, or 7-10 drops per administration. In one example, the formulations of the disclosure are administered about one drop per time and 1-6 times per day.

In some embodiments, the compound described herein is formulated into a pharmaceutical composition. In some embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). In some embodiments, a pharmaceutical composition is provided, comprising a compound described herein and a pharmaceutically acceptable carrier.

Provided herein are pharmaceutical compositions comprising a compound described herein and a pharmaceutically acceptable diluent, excipient, or carrier. The compound may be a compound of Formula I or II as described herein. In some embodiments, the compound is administered as pharmaceutical compositions in which one or more compounds, are mixed with other active ingredients, as in combination therapy. In some embodiments, the pharmaceutical compositions include one or more compounds as described herein.

A pharmaceutical composition, as used herein, refers to a mixture of a compound described herein, with other chemical components, such as carriers, stabilizers, diluents, dispersing agents, suspending agents, thickening agents, and/or excipients. The pharmaceutical composition may facilitate administration of the compound to a patient. In some embodiments for practicing the methods of treatment or use provided herein, effective amounts of one or more compounds described herein are administered in a pharmaceutical composition to a patient having a disease or condition to be detected, diagnosed, or treated. In some embodiments, the patient is a human. An effective amount may vary depending on the severity of the disease, the age and relative health of the patient, the potency of the compound used and other factors. The compounds described herein are used singly or in combination with one or more diagnostic or therapeutic agents as components of mixtures.

For administration by injection, a compound described herein may be dispersed in a liquid pharmaceutically acceptable vehicle. The liquid pharmaceutically acceptable vehicle can be any aqueous or non-aqueous vehicle known in the art. Examples of aqueous vehicles include physiological saline solutions, solutions of sugars such as dextrose or mannitol, and pharmaceutically acceptable buffered solutions. In some embodiments, the aqueous vehicle is a physiologically compatible buffer, such as, for example, Hank's solution, Ringer's solution, aqueous acetate buffer, aqueous citrate buffer, aqueous carbonate buffer, aqueous phosphate buffer, aqueous succinate buffer, aqueous lactate buffer, or physiological saline buffer. Examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, surfactants, and other components. The pharmaceutical composition may comprise a cyclodextrin, for example, sulfobutylether β-cyclodextrin or hydroxypropyl β-cyclodextrin.

A pharmaceutical composition of a compound described herein may be for parenteral administration, for example, by injection. A compound for administration by injection may be prepared as, for example, an aqueous or oil suspension or emulsion in an injection medium. The injection medium may comprise castor oil (ricinus oil), castor oil (ethyoxylated), sesame oil, soybean oil, corn oil, cottonseed oil, or peanut oil, as well as elixirs, mannitol, dextrose, benzyl alcohol, PEG 400, ethylene glycol, polysorbate 20, diethylene glycol monoethyl ether, 10% aqueous poloxamer-188, glycerol, 10% aqueous poloxamer-407, or poloxamer 124.

In some embodiments, the pharmaceutical formulation comprises one or more surfactants. A surfactant is a material that is hydrophobic or amphiphilic (i.e., including both a hydrophilic and a hydrophobic component or region). Surfactants can be used to modify the surface properties of a particle and alter the way in which a particle is dispersed, emulsified, or suspended. In some embodiments, the surfactant comprises a lipid. Lipids that may be used include the following classes of lipids: fatty acids and derivatives, mono-, di- and triglycerides, phospholipids, sphingolipids, cholesterol and steroid derivatives, terpenes, prostaglandins and vitamins. Examples of fatty acids include lauric, physeteric, myristoleic, palmitoleic, petroselinic, and oleic acids, and mono, di- and triglycerides thereof. Such mono-, di-, and triglycerides include, for example, digalactosyldiglyceride, 1,2-dioleoyl-sn-glycerol, 1,2-dipalmitoyl-sn-3 succinylglycerol, and 1,3-dipalmitoyl-2-succinylglycerol. In some embodiments, the surfactant comprises a phospholipid. Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Steroids which may be used include cholesterol, cholesterol sulfate, cholesterol hemisuccinate, 6-(5-cholesterol 3β-yloxy) hexyl-6-amino-6-deoxy-1-thio-α-D-galactopyranoside, 6-(5-cholesten-3 β-yloxy)hexyl-6-amino-6-deoxy]-1-thio-α-D mannopyranoside, cholesteryl(4′-trimethylammonio)butanoate, and sodium deoxycholate (NaDOC). Surfactant products include Tween 20, Tween 80, and Neobee M-5.

Other surfactants include ethoxylated sorbitan esters, sorbitan esters, fatty acid salts, sugar esters, pluronics, tetronics, ethylene oxides, butylene oxides, propylene oxides, anionic surfactants, cationic surfactants, mono and diacyl glycerols, mono and diacyl ethylene glycols, mono and diacyl sorbitols, mono and diacyl glycerol succinates, alkyl acyl phosphatides, fatty alcohols, fatty amines and their salts, fatty ethers, fatty esters, fatty amides, fatty carbonates, cholesterol esters, cholesterol amides and cholesterol ethers, aluminum monostearate, ammonium lauryl sulfate, calcium stearate, dioctyl calcium sulfosuccinate, dioctyl potassium sulfosuccinate, dioctyl sodium sulfosuccinate, emulsifying wax, magnesium lauryl sulfate, potassium oleate, sodium castor oil, sodium cetostearyl sulfate, sodium lauryl ether sulfate, sodium lauryl sulfate, sodium lauryl sulfoacetate, sodium oleate, sodium stearate, sodium stearyl fumarate, sodium tetradecyl sulfate, zinc oleate, zinc stearate, benzalconium chloride, cetrimide, cetrimide bromide, and cetylpyridinium chloride.

Oral administration may be another route for administration of the compounds described herein. The pharmaceutical composition may bin the form of, for example, a capsule or an enteric coated tablet. A compound described herein may thus be diluted by an excipient and/or within a carrier. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients, carriers, and vehicles include lactose, dextrose, sucrose, sorbitol, mannitol, starches, gum acacia, calcium phosphate, alginates, tragacanth, gelatin, calcium silicate, microcrystalline cellulose, polyvinylpyrrolidone (PVP), cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

In some embodiments, the compounds described herein are formulated for ocular administration. In some embodiments, the ocular formulations is liquid (in form of solutions, suspensions, powder for reconstitution, sol to gel systems), semi solids (ointments and gels), solids (ocular inserts), and intraocular dosage forms (injections, irrigating solutions and implants).

Provided herein are ophthalmic formulations comprising the compounds described herein and an ophthalmologically acceptable component. The ophthalmic formulation may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix.

By a “pharmaceutically acceptable” or “opthalmologically acceptable” component is meant a component that is not biologically or otherwise undesirable, i.e., the component may be incorporated into an ophthalmic formulation of the disclosure and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration.

Ophthalmic formulations may be adapted for topical administration to the eye in the form of a suspension or emulsion. The ophthalmic formulation may include an ophthalmologically acceptable carrier, Such carriers include, for example, water, mixtures of water, for example, phosphate buffer, boric acid, sodium chloride, and sodium borate, and water-miscible solvents such as lower alcohols, aryl alcohols, polyalkylene glycols, carboxymethylcellulose, polyvinylpyrrolidone, and isopropyl myristate. The ophthalmic formulation may also include one or more excipients such as emulsifying agents, preserving agents, wetting agents, bodying agents. For example, the ophthalmic formulation may include polyethylene glycols 200, 300, 400 and 600, carbowaxes 1,000, 1,500, 4,000, 6,000 and 10,000, antibacterial components such as quaternary ammonium compounds, phenylmercuric salts, thimerosal, methyl and propyl paraben, benzyl alcohol, phenyl ethanol, buffering agents such as sodium borate, sodium acetates, gluconate buffers, and other agents such as sorbitan monolaurate, triethanolamine, oleate, polyoxyethylene sorbitan monopalmitylate, dioctyl sodium sulfosuccinate, monothioglycerol, thiosorbitol, and ethylenediamine tetracetic acid. The ophthalmic formulation may be isotonic. The ophthalmic formulation may also include a surfactant or a stabilizer. Surfactants include Carbopol®. Stabilizers include sodium bisulfite, sodium metabisulfate and sodium thiosulfate.

The formulation may include an effective amount of a permeation enhancer that facilitates penetration of the formulation components through cell membranes, tissues, and extra-cellular matrices, including the cornea. The “effective amount” of the permeation enhancer represents a concentration that is sufficient to provide a measurable increase in penetration of one or more of the formulation components through membranes, tissues, and extracellular matrices as just described. Suitable permeation enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

### Kits and Packages

Provided herein is a kit that includes a compound described herein, an imaging device, and optionally suitable packaging. The imaging device may be a retinal imaging device. In some embodiments, the kit further includes instructions for use.

The imaging device may include lens(es) and image sensors for detecting a signal emitted. In some embodiments, the imaging device detects a fluorescent signal. In some embodiments, the imaging device further includes a laser light source which can be used to activate the fluorescent signal. The imaging device may comprise a suitable retina scanner.

Table of Acronyms and Abbreviations

| Abbreviation | Meaning |
|---|---|
| Aβ | Amyloid Beta |
| BINAP | (2,2′-bis(diphenylphosphino)- |
| | 1,1′-binaphthyl) |
| DIBAL-H | Diisobutylaluminium hydride |
| DMF | Dimethylformamide |
| Et | Ethyl |
| Me | Methyl |
| Min | Minute |
| PCC | Pyridinium chlorochromate |
| THF | Tetrahydrofuran |

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

General Information: NMR spectra were recorded on a Bruker 300 MHz spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on a Waters ZQ2000 Mass Spectrometer with electrospray ionization.

### Example 1

### Synthesis of (E)-6-(2-(2-hydroxyethoxy)ethoxy)-3-oxo-2-((6-(piperidin-1-yl)naphthalen-2-yl)methylene)hexanenitrile (1)

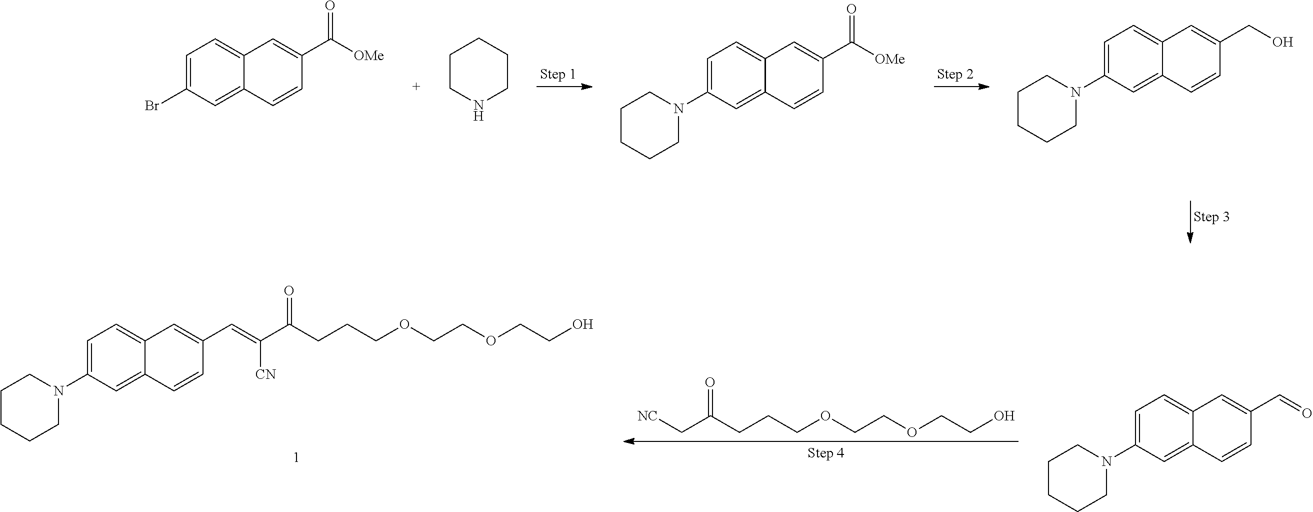

Coupling of methyl 6-bromo-2-naphthaoate with pyrrolidine is carried out in step 1 using palladium acetate/BINAP and CS₂CO₃ as a base and toluene as a solvent. Upon refluxing for about 30 hours, methyl 6-(piperidin-1-yl)-2-naphthoate is formed. Reduction with lithium aluminum hydride in step 2 followed by oxidation with MnO₂ in step 3 provides 6-(piperidin-1-yl)-2-naphthaldehyde. Coupling of 6-(piperidin-1-yl)-2-naphthaldehyde with 6-(2-(2-hydroxyethoxy)ethoxy)-3-oxohexanenitrile in step 4 using piperidine as a base in THF for about 24 hours provides compound 1.

¹H NMR (600 MHz, CDCl₃): δ 8.31-8.22 (m, 2H), 8.14 (dd, J=8.8, 1.8 Hz, 1H), 7.79 (d, J=9.2 Hz, 1H), 7.68 (d, J=8.8 Hz, 1H), 7.32 (dd, J=9.2, 2.5 Hz, 1H), 7.08 (d, J=2.3 Hz, 1H), 3.82-3.74 (m, 2H), 3.70 (dd, J=5.8, 3.4 Hz, 2H), 3.66-3.56 (m, 6H), 3.50-3.35 (m, 4H), 3.05 (t, J=7.1 Hz, 2H), 2.38 (t, J=6.3 Hz, 1H), 2.12-1.98 (m, 2H), 1.83-1.66 (m, 6H).

LC-MS (m/z)=459.1 [M+Na]⁺

Similar compounds are prepared using this synthetic method by using other heterocycles instead of pyrrolidine in step 1, such as optionally substituted piperidine, azetidine, aziridine, etc. Also, other analogs are obtained starting with naphthalene(s) where bromo group is at a different position in step 1 and as such the heterocycle is added at different positions on the naphthalene. Also, similar compounds with different side chains are prepared by adding different cyano compound in step 4 such as the ones shown below:

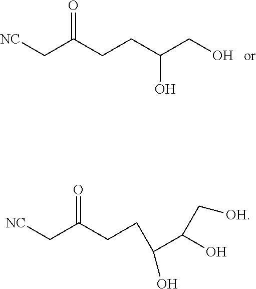 [SMILES: N#CCC(=O)CCC(O)CO ; N#CCC(=O)CCC(O)C(O)CO]

Accordingly, Example 1a was synthesized by the method provided above, using 6,7-dihydroxy-3-oxoheptanenitrile in step 4 to provide (E)-6,7-dihydroxy-3-oxo-2-((6-(piperidin-1-yl)naphthalen-2-yl)methylene)heptanenitrile:

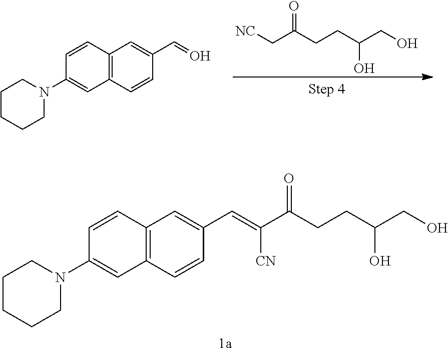 [SMILES: O=Cc1ccc2cc(N3CCCCC3)ccc2c1 ; N#CCC(=O)CCC(O)CO ; N#C/C(=C\c1ccc2cc(N3CCCCC3)ccc2c1)C(=O)CCC(O)CO ; "1a"]

¹H NMR (600 MHz, CDCl₃) δ 8.29 (s, 1H), 8.27 (s, 1H), 8.14 (dd, J=8.8, 1.7 Hz, 1H), 7.79 (d, J=9.2 Hz, 1H), 7.68 (d, J=8.8 Hz, 1H), 7.32 (dd, J=9.2, 2.4 Hz, 1H), 7.07 (d, J=2.2 Hz, 1H), 3.82 (qd, J=8.3, 4.3 Hz, 1H), 3.77-3.69 (m, 1H), 3.57-3.53 (m, 1H), 3.48-3.43 (m, 4H), 3.15 (td, J=6.8, 1.2 Hz, 2H), 2.49 (d, J=4.8 Hz, 1H), 1.99-1.87 (m, 3H), 1.80-1.74 (m, 4H), 1.73-1.68 (m, 2H).

LC/MS (m/z)=379.1 [M+H]⁺

### Example 2

### Synthesis of (Z)-6-(2-(2-hydroxyethoxy)ethoxy)-3-oxo-2-(2,2,2-trifluoro-1-(6-(piperidin-1-yl)naphthalen-2-yl)ethylidene)hexanenitrile (2) and (E)-6-(2-(2-hydroxyethoxy)ethoxy)-2-((6-(2-methylpiperidin-1-yl)naphthalen-2-yl)methylene)-3-oxohexanenitrile (3)

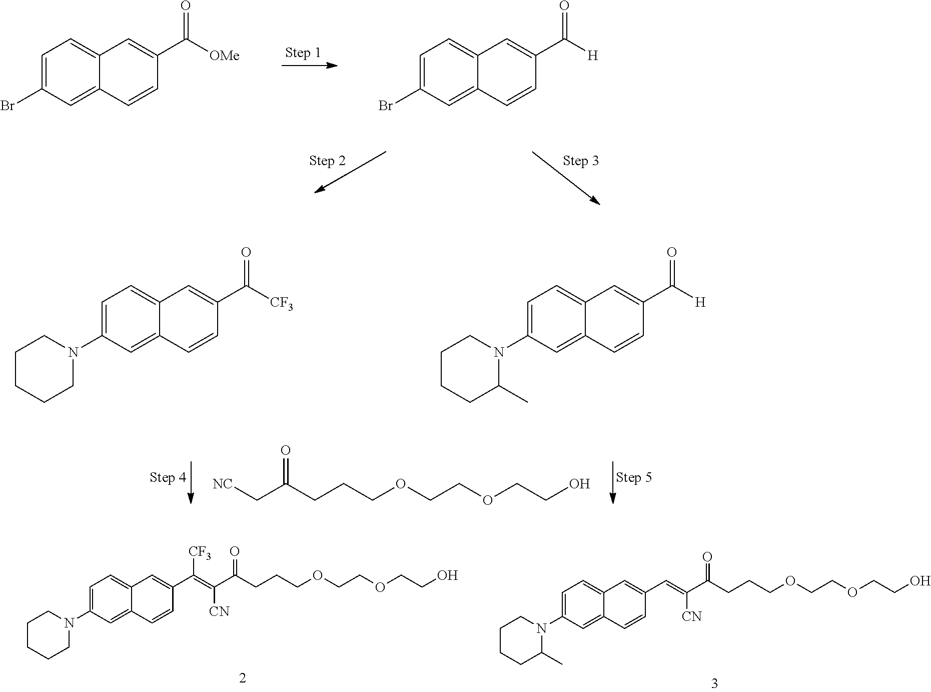

Reduction of methyl 6-bromo-2-naphthaoate with DIBAL-H in THF followed by oxidation with PCC in methylene chloride in step 1 provides 6-bromo-2-naphthaldehyde. Coupling of methyl 6-bromo-2-naphthaldehyde with piperidine is carried out in step 2 using palladium acetate/BINAP and CS₂CO₃ as a base and toluene as a solvent, followed by a nucleophilic reaction with (trifluoromethyl)trimethyl silane and oxidation to provide 2,2,2-trifluoro-1-(6-(piperidin-1-yl)naphthalen-2-yl)ethan-1-one. Coupling of 2,2,2-trifluoro-1-(6-(piperidin-1-yl)naphthalen-2-yl)ethan-1-one with 6-(2-(2-hydroxyethoxy)ethoxy)-3-oxohexanenitrile in step 4 using piperidine as a base in THF provides compound 2.

Alternatively, coupling of methyl 6-bromo-2-naphthaldehyde with 2-methyl piperidine is carried out in step 3 using palladium acetate/BINAP and CS₂CO₃ as a base and toluene as a solvent, to provide 6-(2-methylpiperidin-1-yl)-2-naphthaldehyde. Coupling of 6-(2-methylpiperidin-1-yl)-2-naphthaldehyde with 6-(2-(2-hydroxyethoxy)ethoxy)-3-oxohexanenitrile in step 5 using piperidine as a base in THF provides compound 3.

Similar compounds are prepared using this synthetic method by using other bases instead of pyrrolidine, such as optionally substituted piperidine, azetidine, aziridine, etc. Also, other analogs are obtained starting with naphthalene(s) where bromo group is at a different position in step 1 and as such the heterocycle is added at different positions on the naphthalene. Also, similar compounds with different side chains are prepared by adding different cyano compounds in step 4 such as the ones shown below:

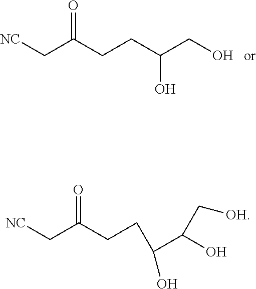 [SMILES: N#CCC(=O)CCC(O)CO ; N#CCC(=O)CCC(O)C(O)CO]

Following the procedures set forth above but using 8-bromo-2-naphthaldehyde as a starting material, the following compounds can be prepared:

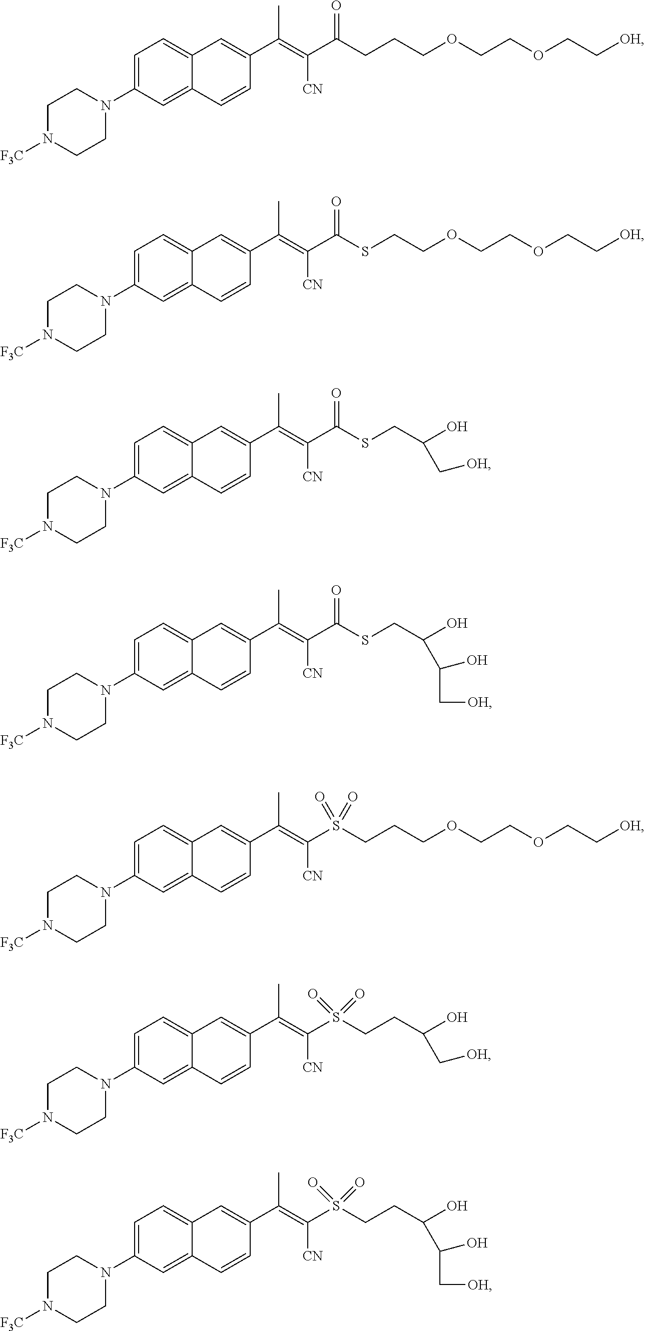

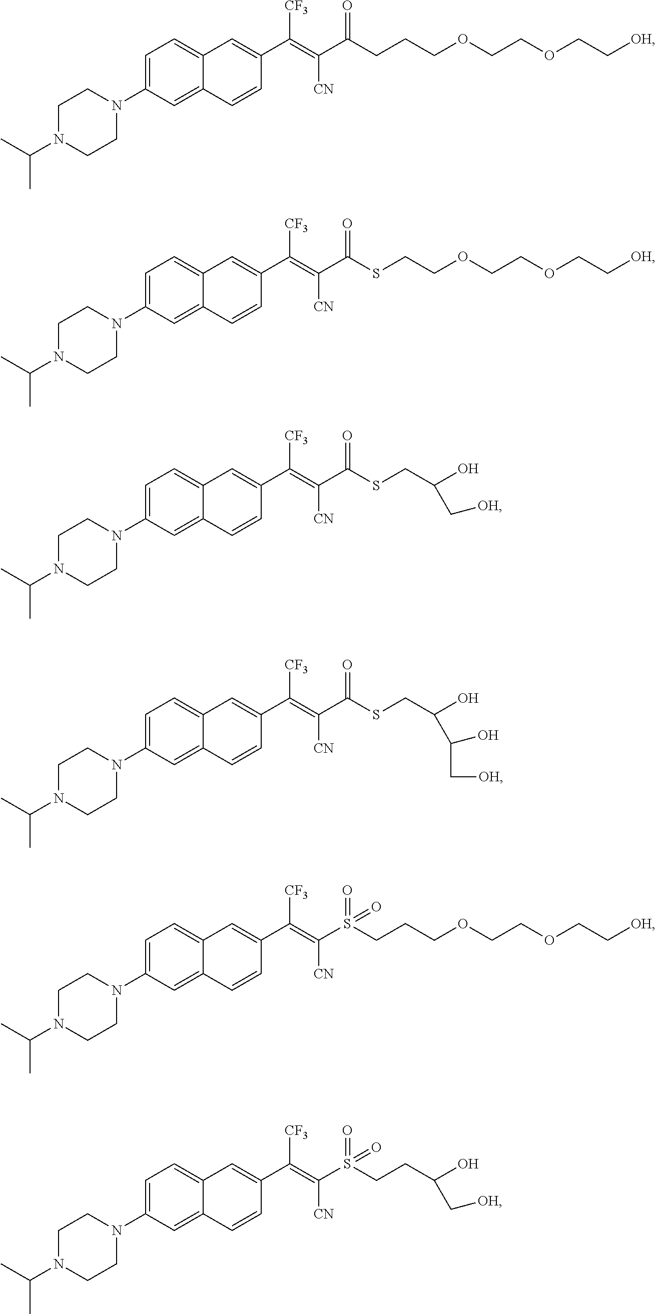 [SMILES: CC(C)N1CCN(c2ccc3cc(/C(=C(\C#N)C(=O)CCCOCCOCCO)C(F)(F)F)ccc3c2)CC1 ; CC(C)N1CCN(c2ccc3cc(/C(=C(\C#N)C(=O)SCCOCCOCCO)C(F)(F)F)ccc3c2)CC1 ; CC(C)N1CCN(c2ccc3cc(/C(=C(\C#N)C(=O)SCC(O)CO)C(F)(F)F)ccc3c2)CC1 ; CC(C)N1CCN(c2ccc3cc(/C(=C(\C#N)C(=O)SCC(O)C(O)CO)C(F)(F)F)ccc3c2)CC1 ; CC(C)N1CCN(c2ccc3cc(/C(=C(\C#N)S(=O)(=O)CCCOCCOCCO)C(F)(F)F)ccc3c2)CC1 ; CC(C)N1CCN(c2ccc3cc(/C(=C(\C#N)S(=O)(=O)CCC(O)CO)C(F)(F)F)ccc3c2)CC1]

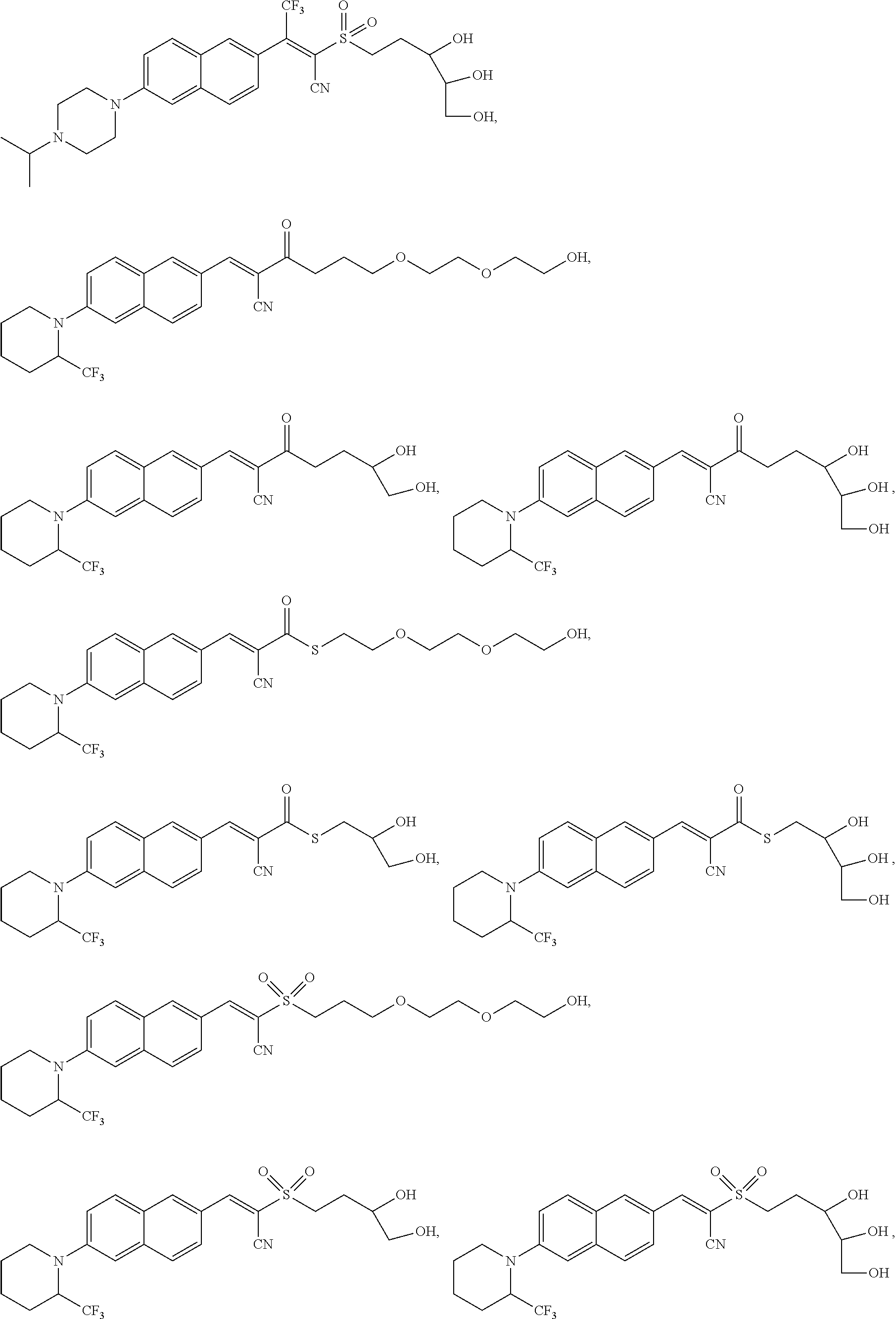

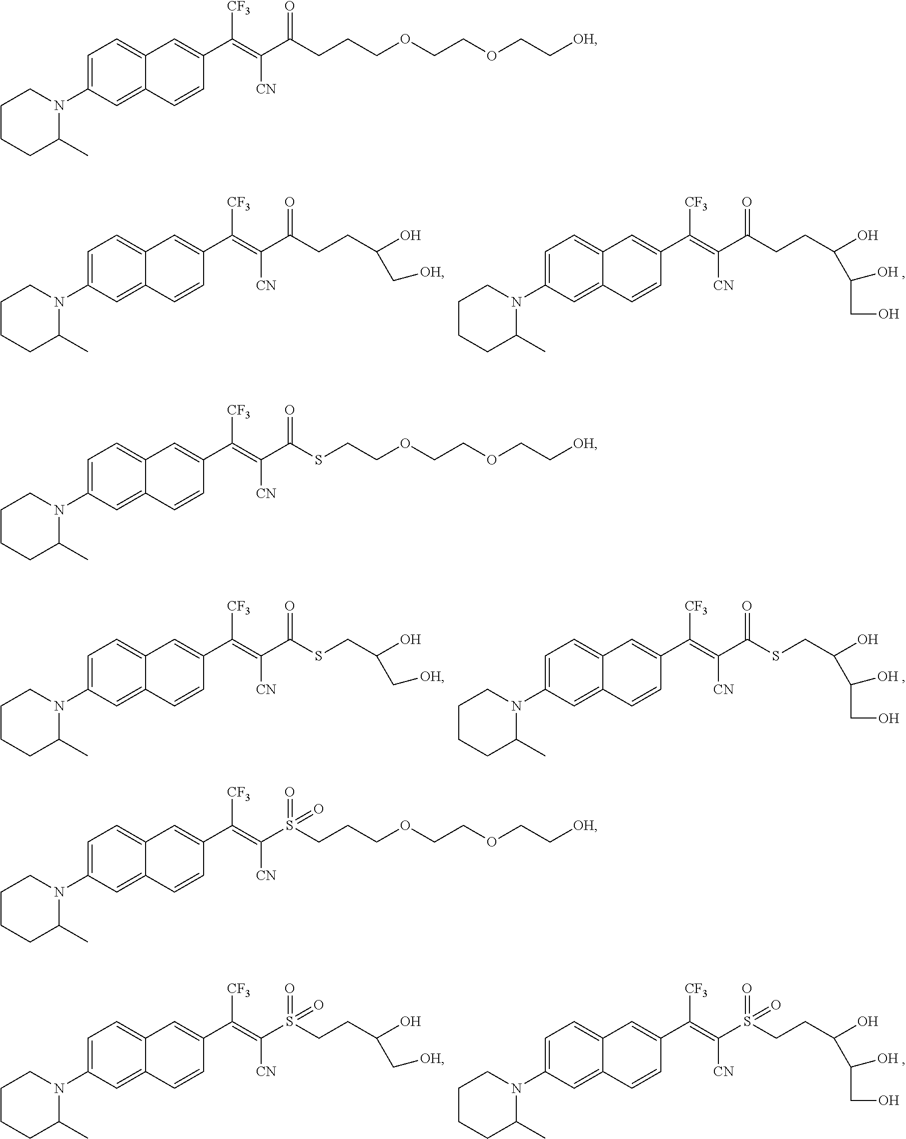

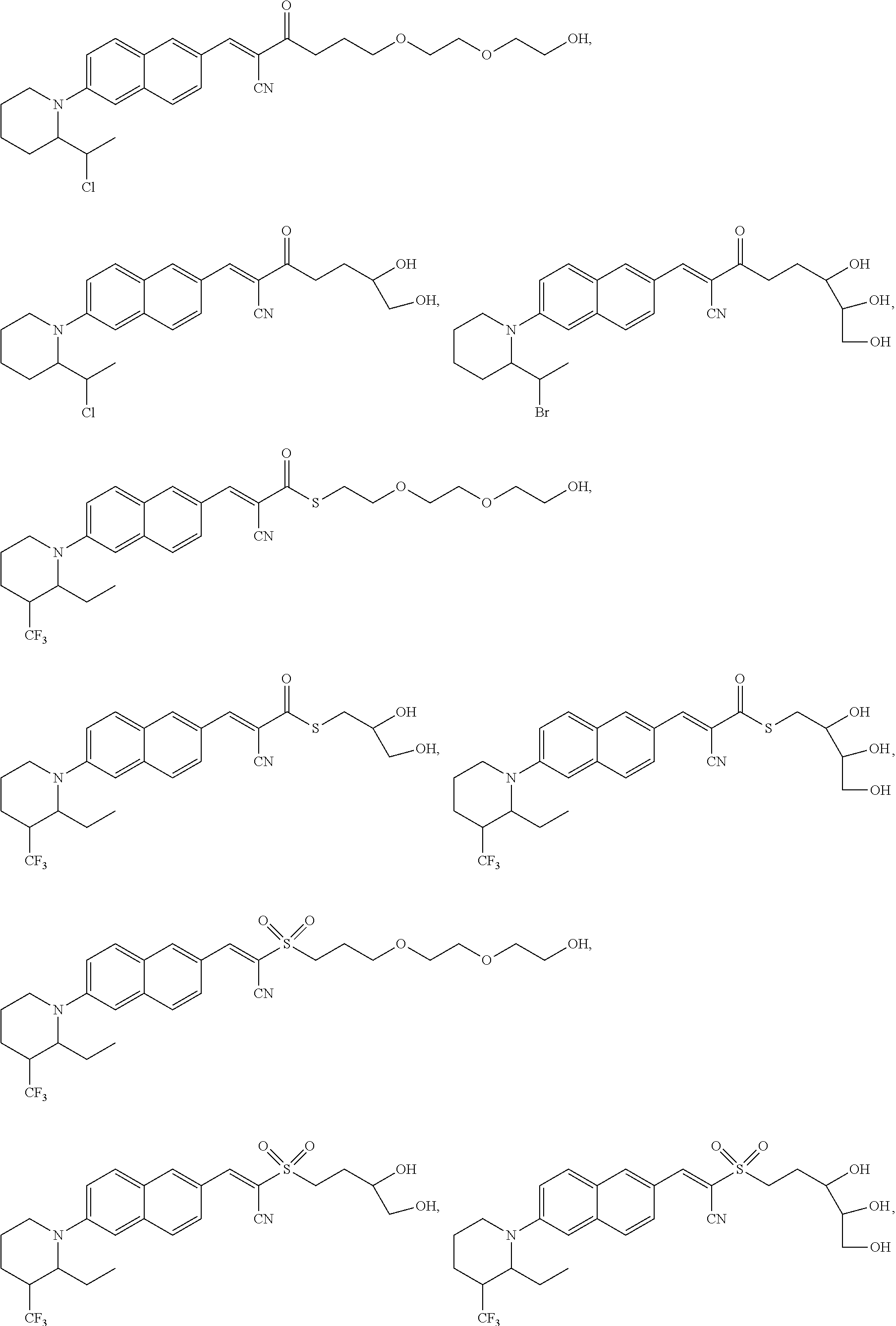

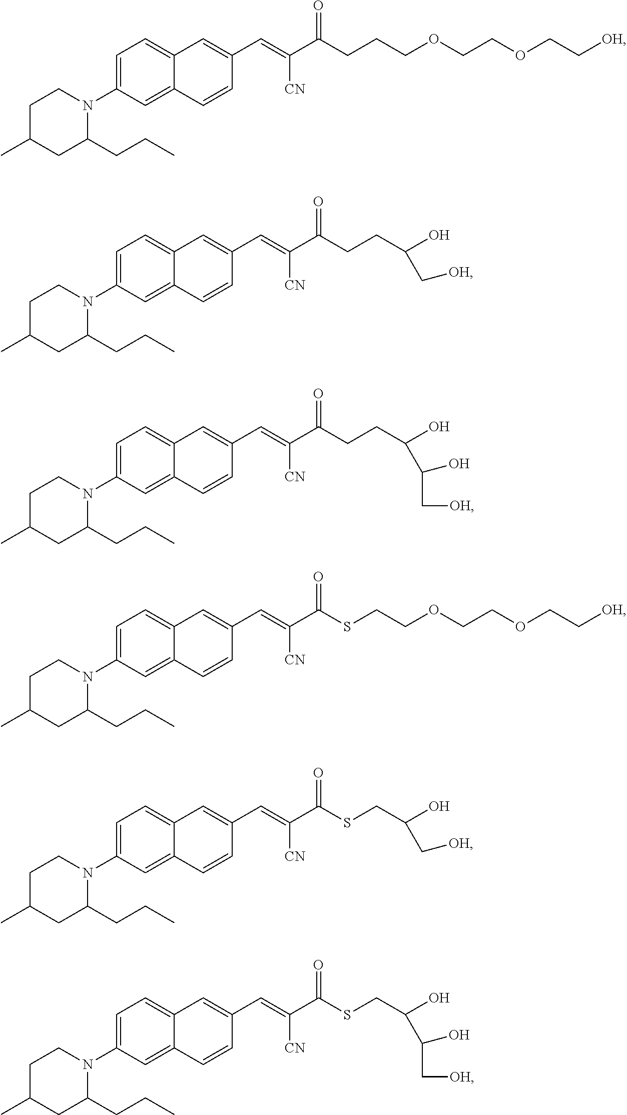 [SMILES: CCCC1CC(C)CCN1c1ccc2cc(/C=C(\C#N)C(=O)CCCOCCOCCO)ccc2c1 ; CCCC1CC(C)CCN1c1ccc2cc(/C=C(\C#N)C(=O)CCC(O)CO)ccc2c1 ; CCCC1CC(C)CCN1c1ccc2cc(/C=C(\C#N)C(=O)CCC(O)C(O)CO)ccc2c1 ; CCCC1CC(C)CCN1c1ccc2cc(/C=C(\C#N)C(=O)SCCOCCOCCO)ccc2c1 ; CCCC1CC(C)CCN1c1ccc2cc(/C=C(\C#N)C(=O)SCC(O)CO)ccc2c1 ; CCCC1CC(C)CCN1c1ccc2cc(/C=C(\C#N)C(=O)SCC(O)C(O)CO)ccc2c1]

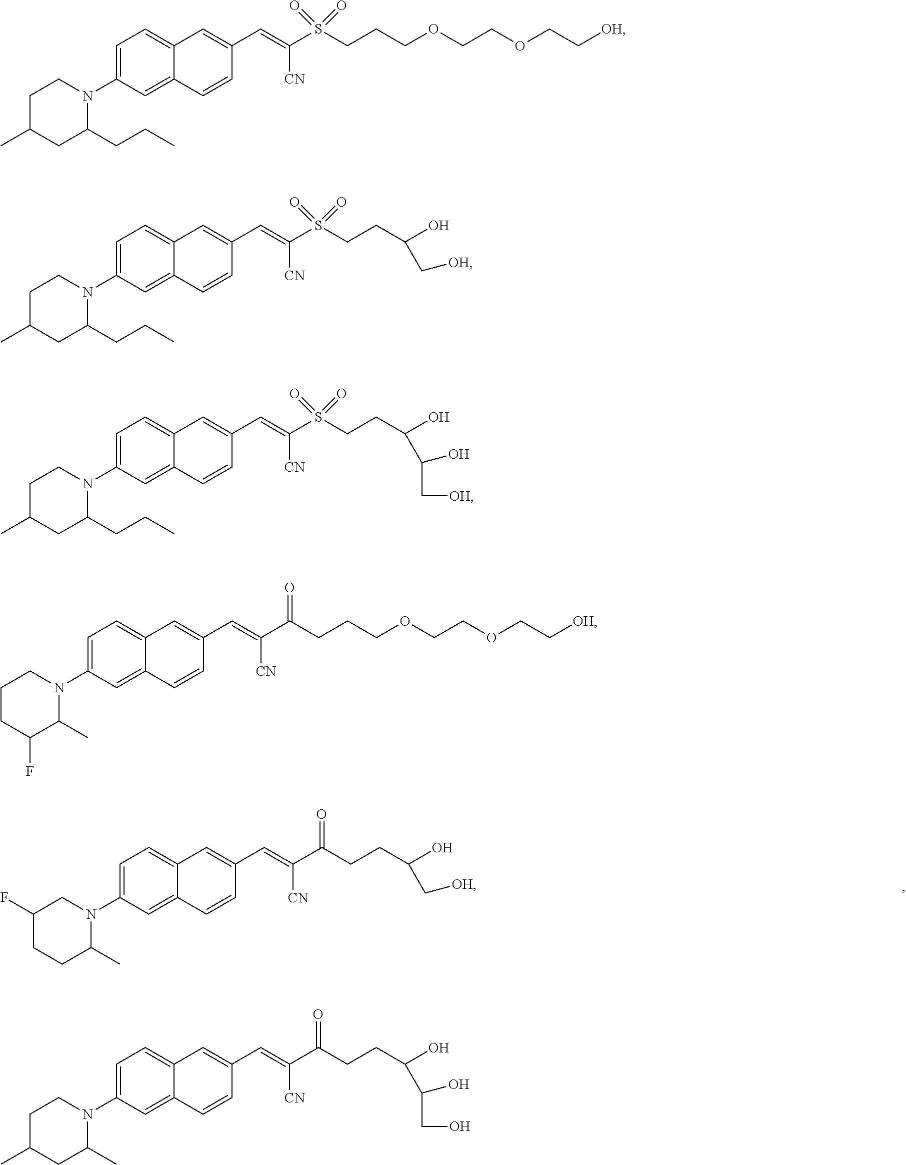 [SMILES: CCCC1CC(C)CCN1c1ccc2cc(/C=C(\C#N)S(=O)(=O)CCCOCCOCCO)ccc2c1 ; CCCC1CC(C)CCN1c1ccc2cc(/C=C(\C#N)S(=O)(=O)CCC(O)CO)ccc2c1 ; CCCC1CC(C)CCN1c1ccc2cc(/C=C(\C#N)S(=O)(=O)CCC(O)C(O)CO)ccc2c1 ; CC1C(F)CCCN1c1ccc2cc(/C=C(\C#N)C(=O)CCCOCCOCCO)ccc2c1 ; CC1CCC(F)CN1c1ccc2cc(/C=C(\C#N)C(=O)CCC(O)CO)ccc2c1 ; CC1CCN(c2ccc3cc(/C=C(\C#N)C(=O)CCC(O)C(O)CO)ccc3c2)C(C)C1]

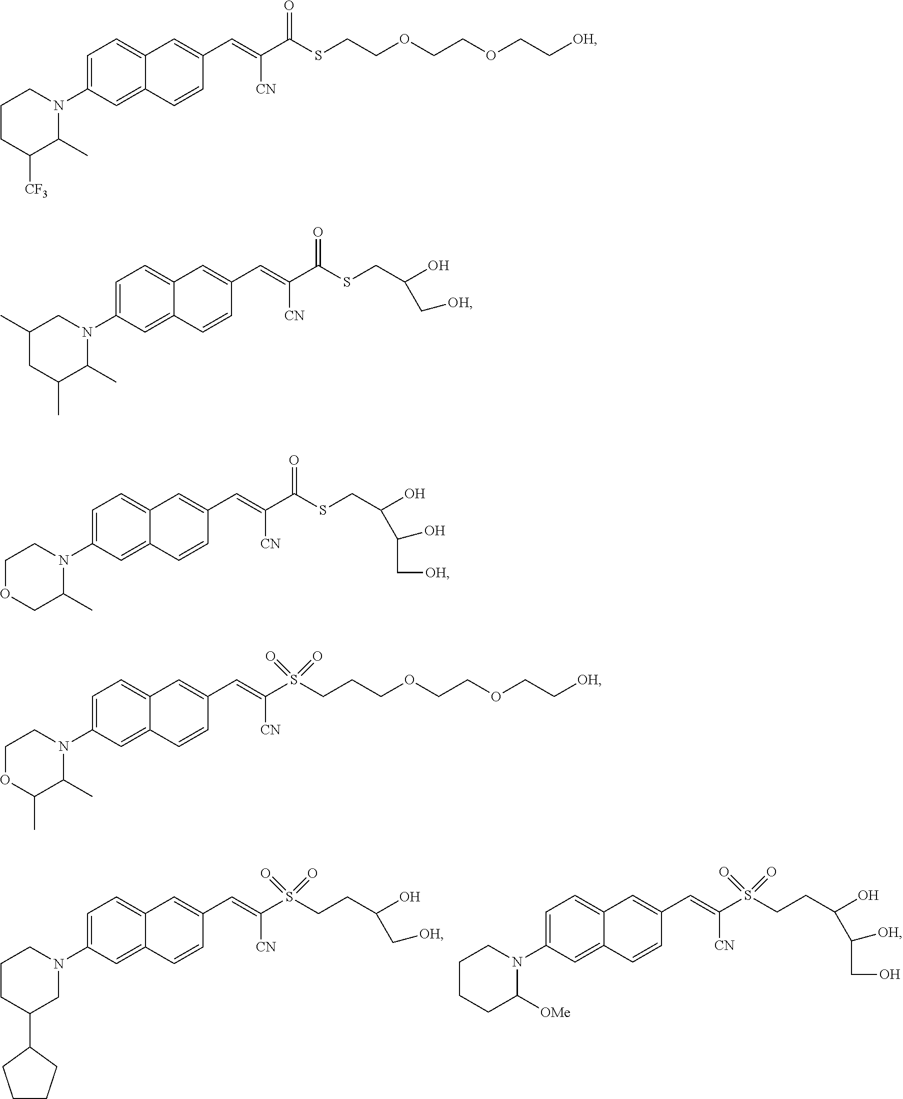

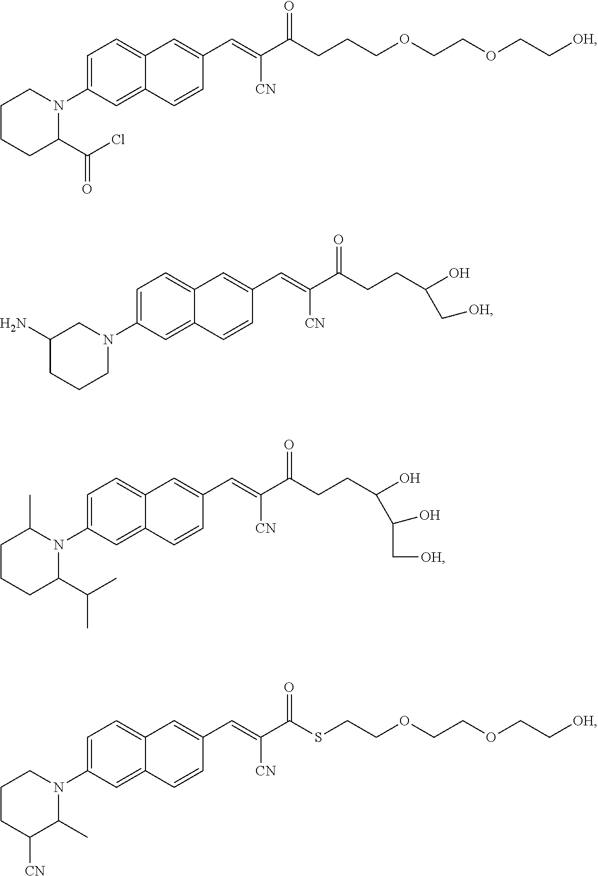 [SMILES: N#C/C(=C\c1ccc2cc(N3CCCCC3C(=O)Cl)ccc2c1)C(=O)CCCOCCOCCO ; N#C/C(=C\c1ccc2cc(N3CCCC(N)C3)ccc2c1)C(=O)CCC(O)CO ; CC(C)C1CCCC(C)N1c1ccc2cc(/C=C(\C#N)C(=O)CCC(O)C(O)CO)ccc2c1 ; CC1C(C#N)CCCN1c1ccc2cc(/C=C(\C#N)C(=O)SCCOCCOCCO)ccc2c1]

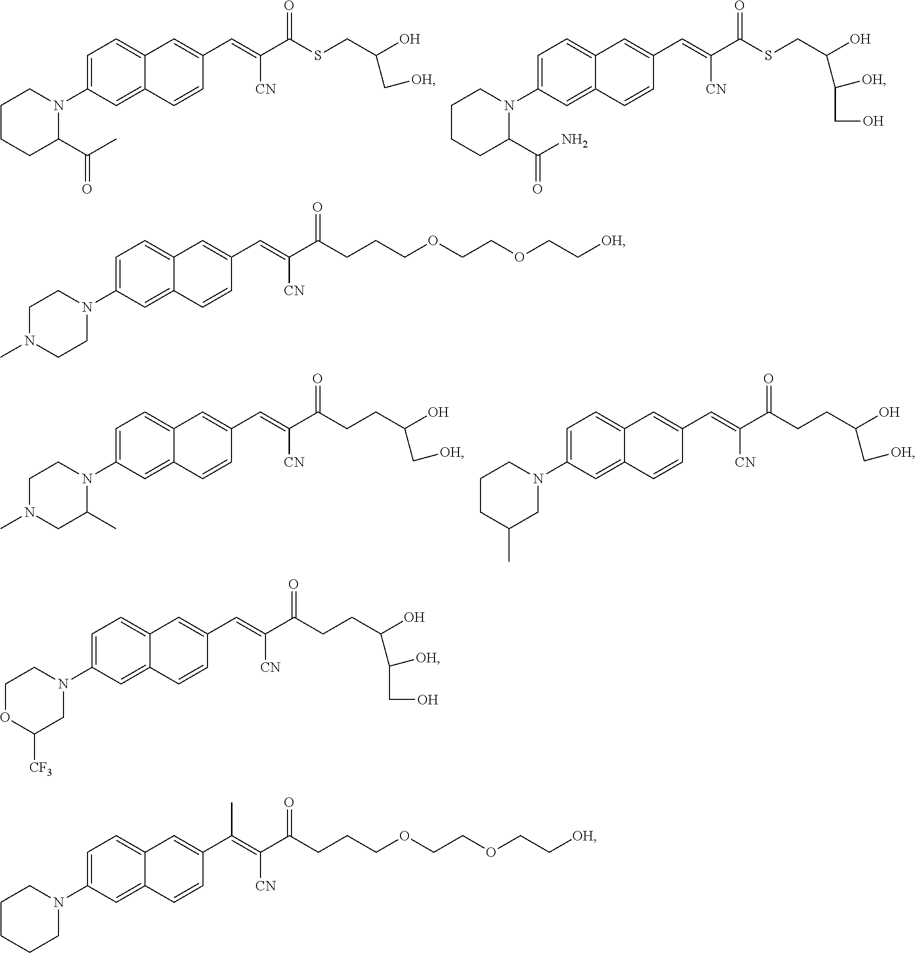

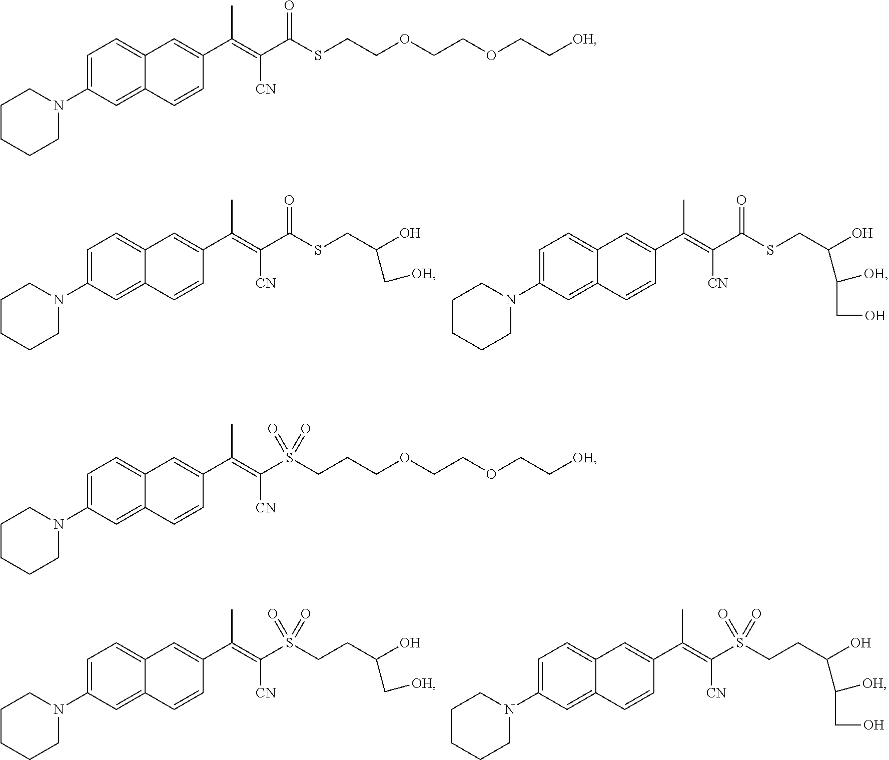

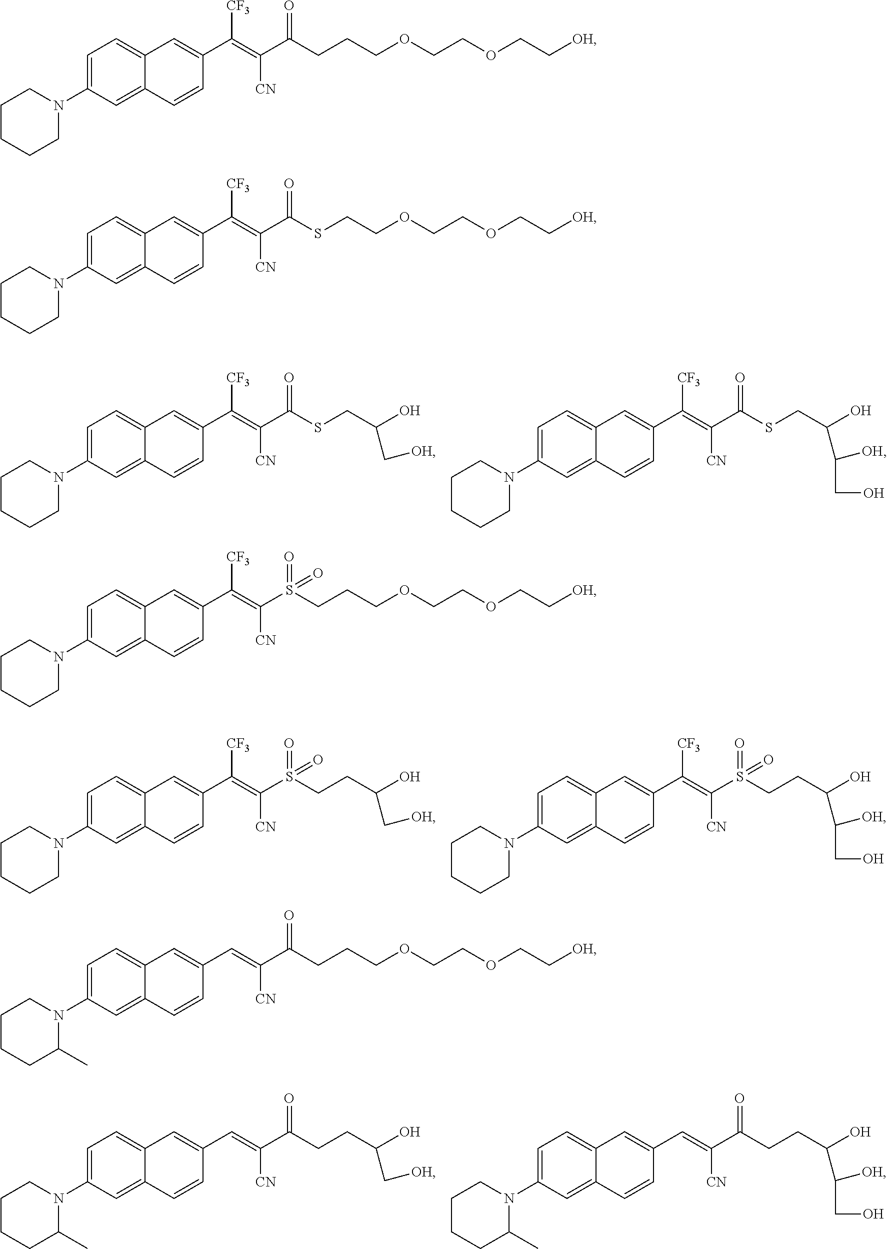

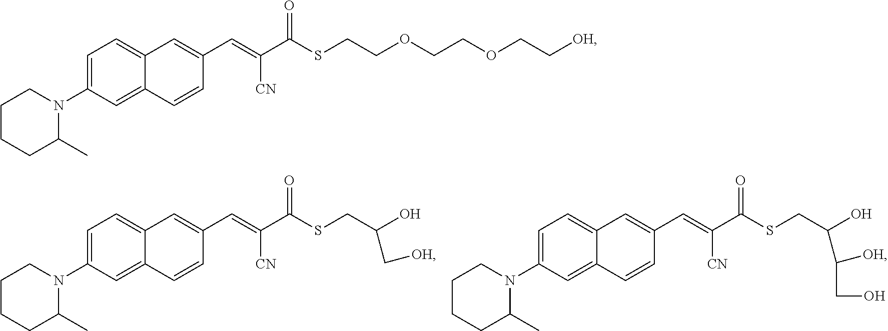

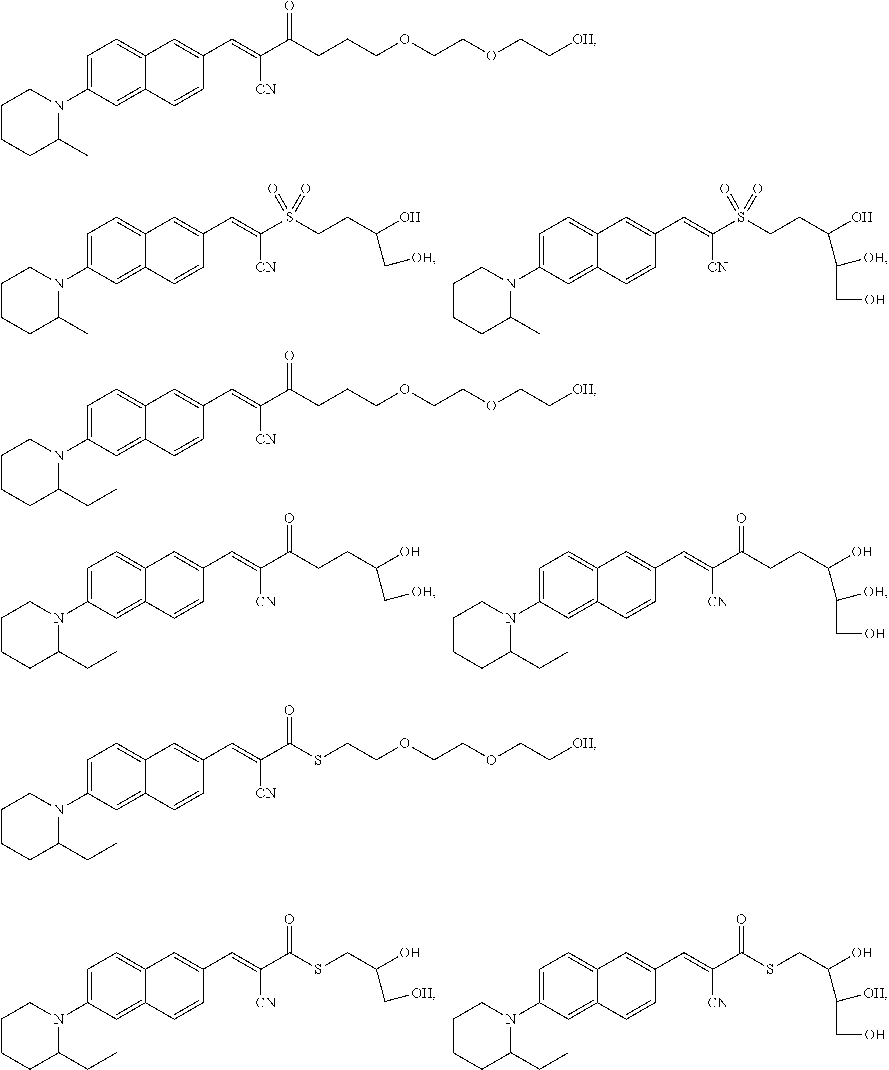

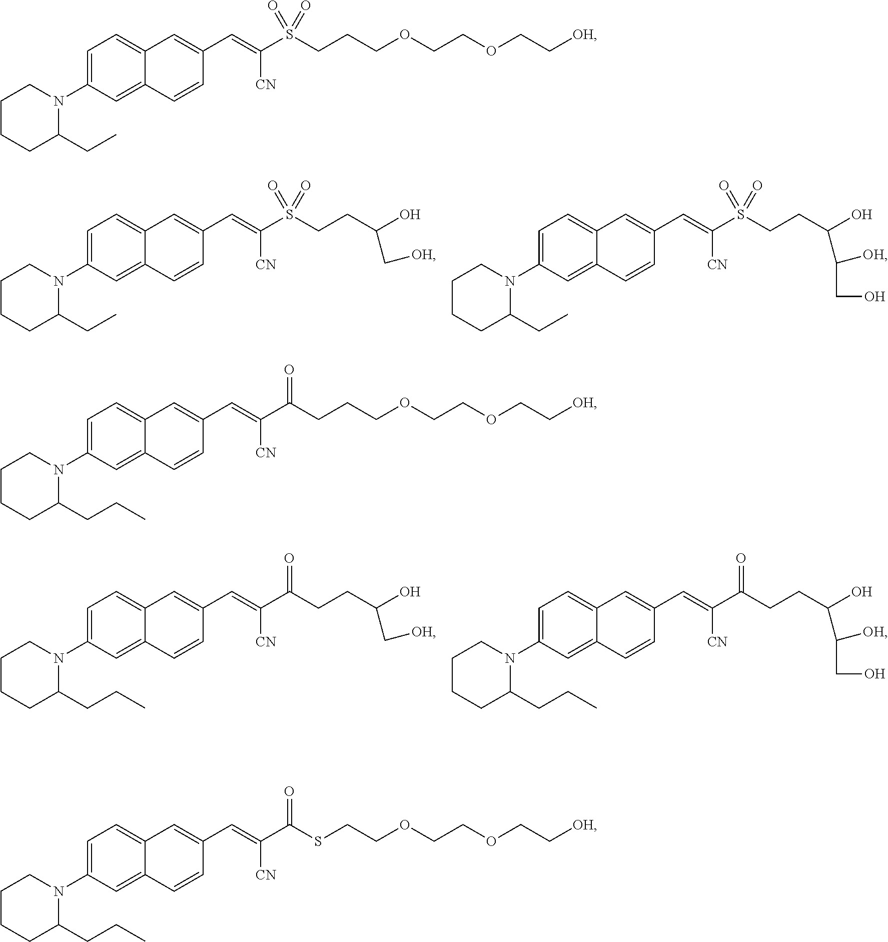

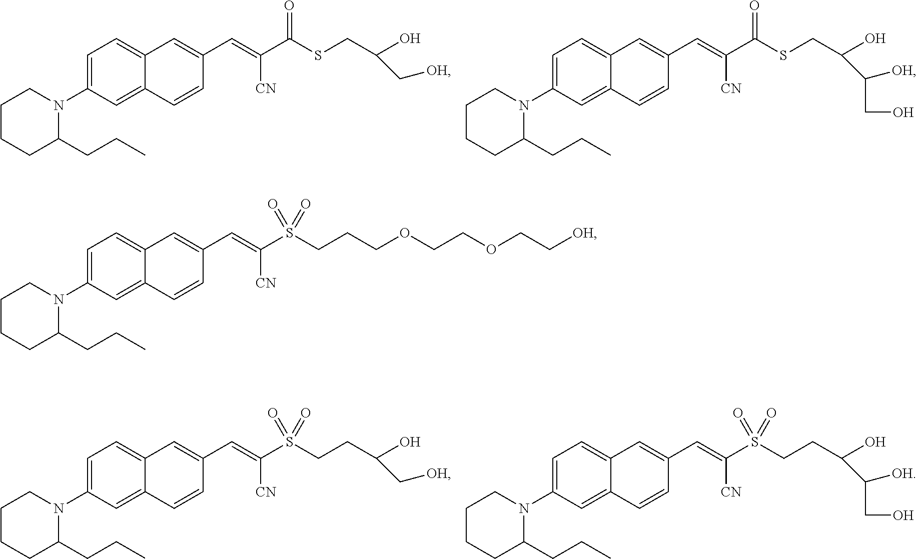 [SMILES: CCCC1CCCCN1c1ccc2cc(/C=C(\C#N)C(=O)SCC(O)CO)ccc2c1 ; CCCC1CCCCN1c1ccc2cc(/C=C(\C#N)C(=O)SCC(O)C(O)CO)ccc2c1 ; CCCC1CCCCN1c1ccc2cc(/C=C(\C#N)S(=O)(=O)CCCOCCOCCO)ccc2c1 ; CCCC1CCCCN1c1ccc2cc(/C=C(\C#N)S(=O)(=O)CCC(O)CO)ccc2c1 ; CCCC1CCCCN1c1ccc2cc(/C=C(\C#N)S(=O)(=O)CCC(O)C(O)CO)ccc2c1]

### Example 3

Model mice having a detectable target protein, for example, an amyloid beta protein, or a phosphorylated tau protein, or an accumulated mass thereof, are administered a representative compound of formula I or II as described herein intravenously. Retinas of the mice are removed and are mounted on slides. The retinas are washed with PBS 2 times for 5 minutes. A solution of 98% formic acid is added—5 minutes for antigen retrieval. The sampled are washed with distilled water—2 times for 5 minutes. The samples are equilibrated in 1×PBS for 15 minutes, then blocked with 10% goat/donkey serum in 1×PBS (depending on antibodies) for 1 hour. Samples are covered in foil, and washed with 1×PBS 3 times for 5 minutes each. Samples are stained with DAPI (300 nM or 100 ng/mL) in dark for 10 minutes, then tissues are washed 3×10 minutes with PBS. Antifade DAKO mounting medium is added, and coverslip and kept under foil until imaging.

Fluorescent imaging study of the samples is conducted on a Leica DMI 4000B microscope (Leica, Germany) equipped with a TCS SPE camera and Leica 10, 20 and 40× objectives. The following lasers are used to visualize fluorescent probes pertaining to DAPT (blue, nuclear stain), representative compound of formula I or II (green) and hyperphosphorylated tau (red): 408, 488 and 568 nm. Z-stacked images are taken at 40× at 0.5 μm increments to visualize the entire thickness of the tissue. Hyperphosphorylated three repeat tau protein is detected in the retina, producing a detectable fluorescent signal, while in wild type mice of comparable age, no immunostaining with three repeat tau antibody is detected. In conclusion, the study indicates that compounds of formula I or II as described herein can be used as a diagnostic agent for detecting amyloid beta protein or hyperphosphorylated tau protein.

### Example 4

### In Vivo Imaging Studies in Tau Transgenic Mice

This example was conducted to determine whether the compounds of formula I or II as described herein detect Aβ in the retina of a TBI mouse model.

Compound 1 and compound 1a were administered by intravenous injection (15 mg/kg) in anesthetized mice (8-12 month old females) and real-time fluorescence images were collected with a Phoenix Micron IV fluorescence rodent retinal-imaging camera at the following time points: 0, 5 seconds, 10 seconds, 30 seconds, 45 seconds, 10 minutes, 24 hours.

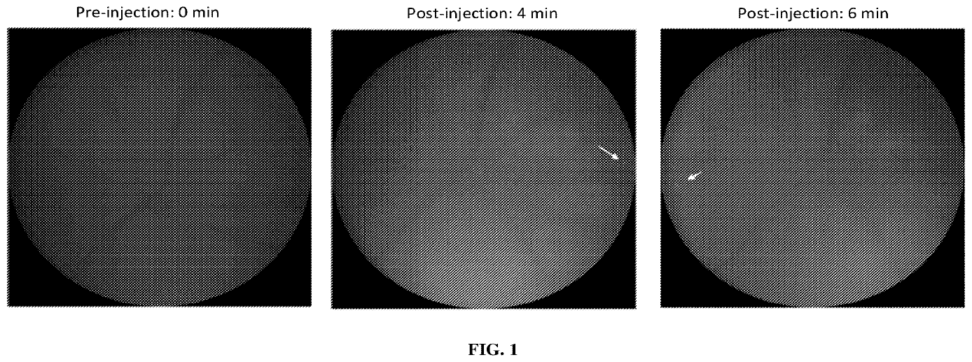

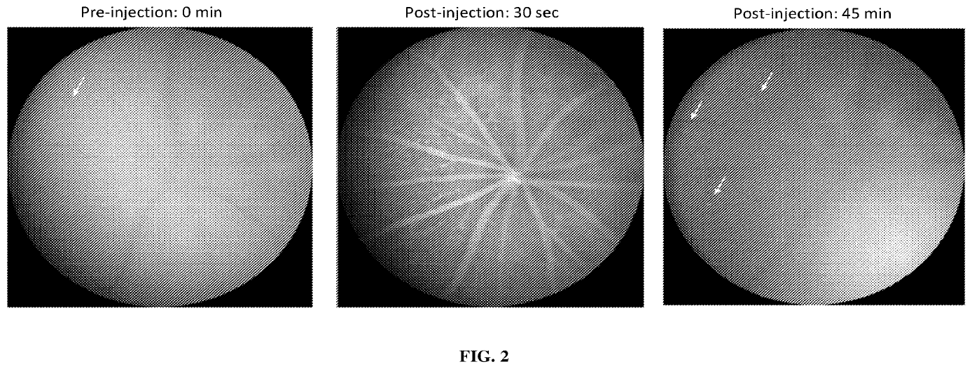

FIG. 1 shows the in vivo retinal image of 3R Tau mouse following IV injection of compound 1 at 15 mg/kg. FIG. 2 shows the in vivo retinal image of 3R Tau mouse following IV injection of compound 1a at 15 mg/kg.

Both Compound 1 and Compound 1a were observed in the retinal vasculature rapidly after tail vein injections (1-2 minutes). Hyperfluorescent signals (indicated by the white arrows) were observed post-injection, especially at the periphery of the retina.

Alternatively, or in addition to retinal scanning, retinal tissues are removed and stained with an anti-Aβ antibody (6E10). The mouse that receives a blast injury displays immunoreactivity to Aβ in the retinal tissue where the uninjured mouse displays no reactivity to 6E10. Compounds of formula I or II fluorescently label retinal deposits that are visible upon fluorescent activation, but does not display any fluorescent enhancement in tissue from the uninjured mouse.

### Example 5

### In Vitro Binding Studies of Compounds with Amyloid Beta

The fluorescent properties and emission spectra of compound 1 and compound 1a with aggregated amyloidogenic proteins were characterized as described below.

Emission spectra for compound 1 and compound 1a were collected after incubation of the compounds with and without aggregated amyloidogenic protein at specific excitation wavelengths (450 nm and 488 nm) employed in standard ocular imaging equipment. The fold increase at maximum emission (compound+Aβ/compound) is calculated for each test compound and is shown in Table 1.

### Methodology

Amyloidogenic proteins (synthetic aggregated amyloid beta (Aβ), α-synuclein (ASYN), transthyretin (TTR), TAR DNA-binding protein 43 (TDP43), and phosphorylated tau (TAU)) were aggregated in vitro using the following procedure. The amyloidogenic protein was removed from the freezer and allowed to warm to room temperature, then 1 mg of amyloidogenic protein was dissolved in 215 μL of ddH₂O for about 10-20 mins at room temperature. To the resulting solution, 2 mL of ddH₂O was added to provide a 100 μM stock solution. A 100 μL aliquot was sampled and frozen (non-aggregated sample). The tubes were placed in a thermoshaker at 350 rpm for 3 days. Care was taken to avoid generating bubbles. Protein aggregation was confirmed by measuring binding to Thioflavin T (ThT). Aggregated protein was then portioned into 150 μL aliquots and stored at −80° C.

The fluorescent emission spectra were measured as follows to determine binding of synthetic aggregated amyloid beta (Aβ), α-synuclein (ASYN), transthyretin (TTR), TAR DNA-binding protein 43 (TDP43), and phosphorylated tau (TAU) to test compounds described herein. The Shimadzu fluorimeter was turned on and allowed to warm up for ~60 minutes. The test compounds were then removed from the −20° C. freezer and were allowed to warm to room temperature. The 100 μM aggregated Aβ solution was then removed from the −80° C. freezer and also allowed to warm to room temperature. 250 μM solutions of each test compound in DMSO were then prepared. These were then used to prepare 4 μM solutions of each test compound, with and without 5 μM aggregated Aβ in 1×PBS, in triplicates. Using a cuvette, the fluorescent emission spectra were measured for each test compound+Aβ solution, employing a DMSO/PBS solution as a blank. Each sample was tested at an excitation of 450 nm and 488 nm. Fluorescent emission spectra were then collected for each 4 μM test compound solution alone. The resulting data were used to plot intensity vs. wavelength for each emission scan. The fold increase at maximum emission (compound+Aβ/compound) is calculated for each test compound.

The fold increase and peak emission data are shown in Table 1.

TABLE 1

| | Compound 1 Fold | | Compound I Peak | | Compound 1a | | Compound 1a | |
|---|---|---|---|---|---|---|---|---|
| Aggregated | Increase | | emission | | Fold Increase | | Peak emission | |
| Protein | 450 nm | 488 nm | 450 nm | 488 nm | 450 nm | 488 nm | 450 nm | 488 nm |
| Aβ | 10.16 | 9.11 | 559 | 556 | 9.48 | 19.60 | 554 | 559 |
| ASYN | 105.50 | 109.97 | 552 | 557 | 120.92 | 190.64 | 554 | 554 |
| TTR | 5.18 | 7.21 | 553 | 560 | 4.67 | 7.35 | 550 | 557 |
| TDP43 | 15.33 | 18.32 | 547 | 556 | 23.23 | 20.40 | 551 | 551 |
| 3RTau | 68.04 | 69.31 | 571 | 577 | 84.37 | 103.65 | 573 | 571 |
| 4RTau | 14.35 | 29.25 | 560 | 562 | 22.19 | 22.48 | 557 | 562 |

Both compound 1 and compound 1a successfully bound to all amyloidogenic aggregates tested (Aβ, ASYN, TTR, TDP43, 3RTau, and 4RTau). The binding intensity and efficiency to specific aggregates, as well as the peak maximum emission varied between each compound and excitation wavelength (450 nm or 488 nm). These results suggest that compound 1 and compound 1a can be used to specifically target amyloidogenic aggregates.

### Example 6

### Binding of Test Compounds to Amyloid Beta in Human Tissues

This example is conducted to determine the utilization of compounds described herein to mark Aβ aggregates in the eye for non-invasive detection using standard ocular imaging equipment, and thus facilitate diagnosis and monitoring of Alzheimer's disease.

It is contemplated that compounds described herein will bind to Aβ deposits in human tissues, and undergo an increase in fluorescence emission in a manner similar to that observed in vitro. Compounds described herein will be tested for their ability to bind and fluoresce Aβ in the retina and brain in Alzheimer's disease human tissues. Sections from these tissues will be co-stained with compounds described herein and an antibody specific for Aβ, using the protocol provided below. Immunofluorescent images will be collected and analyzed to determine if signals from compounds described herein correspond to areas of amyloid deposits defined by Aβ staining.

### Tissue Staining Protocol

Tissue sections are deparaffinized and hydrated as follows. Samples are pre-heated at 60° C. for 1 hour. Slides are placed in holders and are treated with the clearing agent xylene (paraffin solvent) and a series of graded EtOH as follows:
i. 100% xylene—5 min
ii. 100% xylene—5 min
iii. 50%/50% xylene/100% EtOH—3 min
iv. 100% EtOH—3 min
v. 95% EtOH—3 min
vi. 70% EtOH—3 min
vii. 50% EtOH—3 min
viii. Water—2×3 min

The antigen is retrieved by incubating in 99% formic acid for 5 minutes, and is then washed with distilled water for 5 minutes. This step is repeated two times. 10 mM citrate buffer pH 6.0 is then pre-heated to boiling.

The slides are placed in the staining chamber with heated citrate buffer for 20 minutes, then are cooled in a water bath. The slides are then washed with distilled water for 5 minutes. This step is repeated two times.

The slides are equilibrated in 1×PBS for 15 minutes, then blocked in 5% Normal goat serum in PBST for 1 hour at room temperature. The slides are incubated in Aβ 6E10 primary antibody O/N at 4° C. (2.5% NGS in PBST). The tissues are then washed 3×10 minutes in PBST wash buffer.

The slides are incubated in secondary antibody (1:500 in PBST) for 1 hour at room temperature, ensuring samples are kept in the dark from this point forward.

The tissues are then washed 3×10 minutes in PBST. The tissues are stained with test compounds described herein (60 μM) for 30 minutes at room temperature as follows. The test compounds described herein are allowed to warm to room temperature (~30 min), then 5 mg of each test compound is dissolved in 3.75 mL DMSO, with the resulting solution being kept in the dark. 100 μL of each compound solution is then diluted in 5 mL PBS, to provided stain solutions for each test compound described herein.

The stained tissues are then washed 3×10 minutes in PBST. The nuclei are then stained with Hoechst (2:1000 from 1 mg/mL dilution in PBS) for 10 minutes. The tissues are then further washed 3×10 minutes in PBST. The tissues are mounted using Prolong Glass mounting media and let dry ON. Finally, the edges are sealed with nail polish.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A compound of formula: 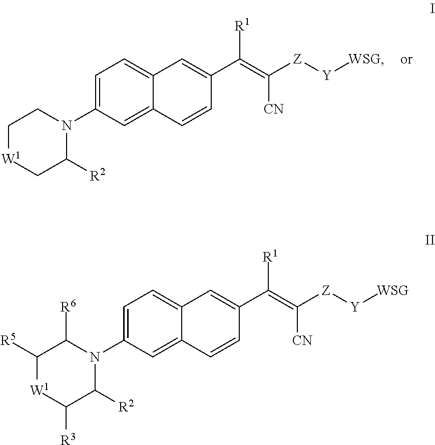
or a pharmaceutically acceptable salt, tautomer or a prodrug thereof wherein:
W¹ is O, N(R¹⁴), or C(R⁴)₂;
R¹⁴ is hydrogen, alkyl, halomethyl having 1 to 3 halo groups, —COOH, —CONH₂, substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is selected from hydrogen, halo, haloalkyl having 1 to 3 halo groups, or cyano;
R², R³, R⁴, R⁵, and R⁶ are each independently hydrogen, halo, haloalkyl having 1 to 3 halo groups, —CN, —C(O)R^{1A}, —C(O)OR^{1A}, —C(O)NR^{1A}R^{1B}, —OR^{1A}, —NHC(O)NR^{1A}R^{1B}, —NR^{1A}R^{1B}, —NHNR^{1A}R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} are each independently hydrogen, halo, haloalkyl having 1 to 3 halo groups, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Z is —C(O)— or —SO₂—; and
Y is CH₂, NH, or S; and
WSG is a water soluble group;
with the proviso that:
when Z is —C(O)—, then Y is not NH,
when Y is CH₂, W¹ is O, N(CH₃), or CH₂ for formula I, then R² is not hydrogen or C₁₋₁₀ alkyl, and
when Y is CH₂, W¹ is O, N(CH₃), or CH₂, and R³, R⁵, R⁶ are hydrogen for formula II, then R² is not hydrogen or C₁₋₁₀ alkyl.

2. The compound of claim 1, having the formula: 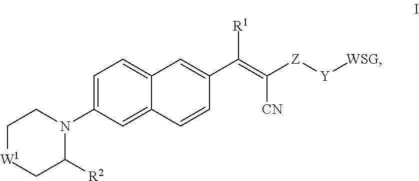
or a pharmaceutically acceptable salt, tautomer or a prodrug thereof.

3. The compound of claim 1, having the formula: 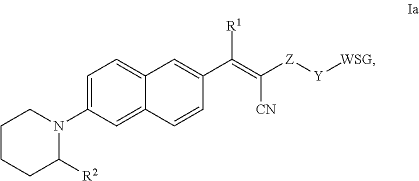
or a pharmaceutically acceptable salt, tautomer or a prodrug thereof.

4. The compound of any one of claims 1-3, wherein R¹ is hydrogen.

5. The compound of any one of claims 1-4, wherein R² is hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, —CN, —OH, —NH₂, —COOH, —CONH₂, —NHNH₂, —NHC(O)NHNH₂, substituted or unsubstituted C₁-C₆ alkyl, or substituted or unsubstituted 2 to 6 membered heteroalkyl.

6. The compound of any one of claims 1-5, wherein R² is hydrogen or unsubstituted C₁-C₄ alkyl.

7. The compound of any one of claims 1-6, wherein R² is unsubstituted methyl, unsubstituted ethyl, or unsubstituted propyl.

8. The compound of claim 1, wherein R³, R⁴, R⁵, and R⁶ are hydrogen.

9. The compound of claim 1, having the formula: 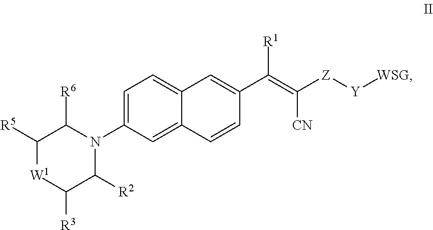
or a pharmaceutically acceptable salt, tautomer or a prodrug thereof.

10. The compound of claim 1, having the formula: 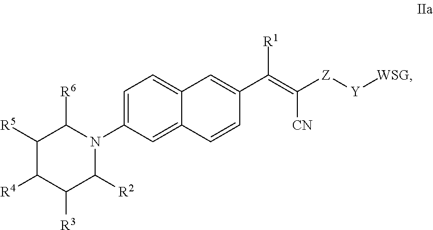
or a pharmaceutically acceptable salt, tautomer or a prodrug thereof.

11. The compound of claim 9 or claim 10, wherein R¹ is hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, or —CN.

12. The compound of claim 9 or claim 10, wherein R¹ is —CF₃ or unsubstituted methyl.

13. The compound of any one of claims 9-12, wherein R², R³, R⁴, R⁵, and R⁶ are hydrogen, halo, —CF₃, —CBr₃, —CCI₃, —CCl₃, —CHF₂, —CHBr₂, —CHCl₂, —CHI₂, —CH₂F, —CH₂Br, —CH₂Cl, —CH₂I, —CN, —OH, —NH₂, —COOH, —CONH₂, substituted or unsubstituted C₁-C₆ alkyl, or substituted or unsubstituted 2 to 6 membered heteroalkyl.

14. The compound of any one of claims 1-13, wherein WSG is selected from the group consisting of: 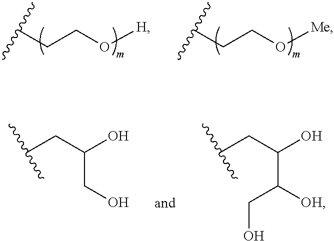
wherein m is an integer having a value from 1-10.

15. The compound of any one of claims 1-13, wherein WSG is selected from the group consisting of: 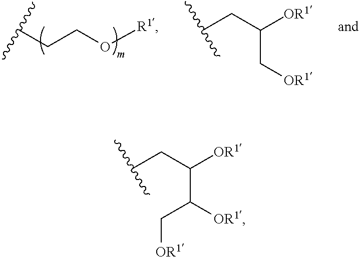
wherein m is an integer having a value from 1-10 and each R^{1′} is independently selected from the group consisting of: 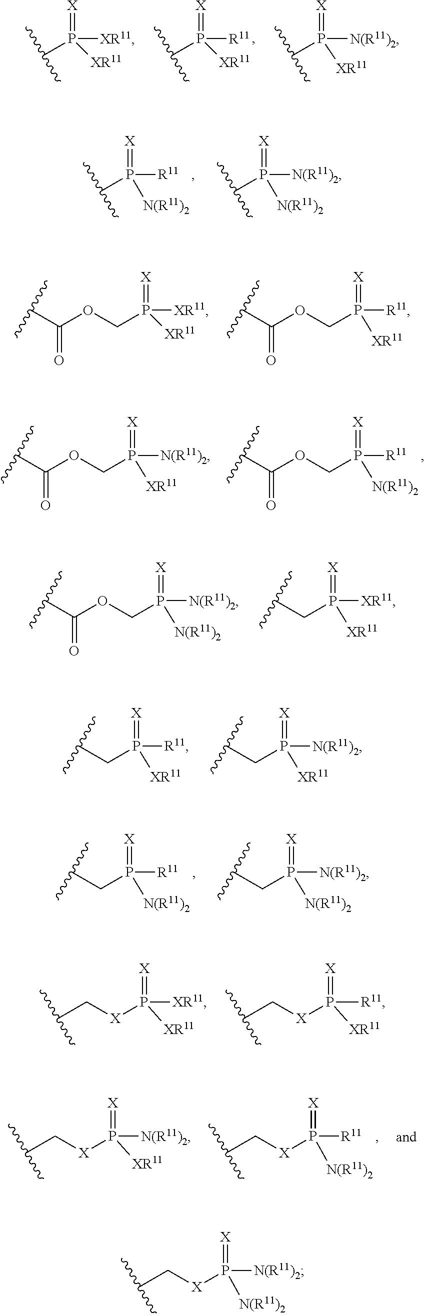
wherein each X is independently O or S;
each R¹¹ is independently selected from hydrogen, C₁₋₁₀ alkyl, C₂₋₆ alkenyl, C₂₋₆ alkynyl, C₃₋₁₀ cycloalkyl, C₆₋₁₀ aryl, 5- to 10-membered heteroaryl, and 4- to 10-membered heterocyclyl; wherein alkyl, alkenyl, alkynyl, cycloalkyl, aryl, heteroaryl, and heterocyclyl are optionally substituted with one to four R²¹; or each XR¹¹ may independently be —XP(X)(R¹²)₂;
each R¹² is independently selected from hydroxy, thiol, —XP(X)(R¹³)₂, C₁₋₁₀ alkyl, —O—C₁₋₁₀ alkyl, and —S—C₁₋₁₀ alkyl;
each R¹³ is independently selected from hydroxy, thiol, C₁₋₁₀ alkyl, —O—C₁₋₁₀ alkyl, and —S—C₁₋₁₀ alkyl;
each R²¹ is independently selected from halo, hydroxy, thiol, —NO₂, —N₃, cyano, C₁₋₁₀ alkyl, C₂₋₆ alkenyl, C₂₋₆ alkynyl, C₃₋₁₀ cycloalkyl, C₁₋₈ haloalkyl, C₆₋₁₀ aryl, 5- to 10-membered heteroaryl, 4- to 10-membered heterocyclyl, —O—C₁₋₁₀ alkyl, —O—C₂₋₆ alkenyl, —O—C₂₋₆ alkynyl, —O—C₃₋₁₀ cycloalkyl, —O—C₁₋₈ haloalkyl, —O-aryl, —O-heteroaryl, —O-heterocyclyl, —NH₂, —NH(R³¹), —N(R³¹)₂, —C(O)(R³¹), —C(O)O(R³¹), —C(O)OH, —C(O)NH₂, —C(O)NH(R³¹), —C(O)N(R³¹)₂, —NHC(O)(R³¹), —NHC(O)O(R³¹), —NHC(O)NH(R³¹), —S(R³¹), —NHS(O)_{y}(R³¹), —N(C₁₋₁₀ alkyl)S(O)_{y}(R³¹), —S(O)_{y}N(R³¹)₂, —S(O)NH(R³¹), and —S(O)_{y}(R³¹);
each R³¹ is independently selected from C₁₋₁₀ alkyl, C₂₋₆ alkenyl, C₂₋₆ alkynyl, C₃₋₁₀ cycloalkyl, C₁₋₈ haloalkyl, aryl, heteroaryl, and heterocyclyl; and
each y is independently 1 or 2.

16. The compound of claim 1, having formula: 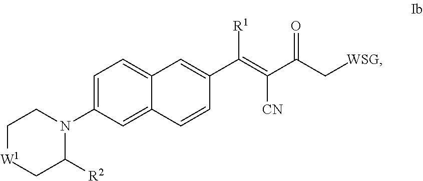
or a pharmaceutically acceptable salt, tautomer or a prodrug thereof.

17. The compound of claim 1, having formula: 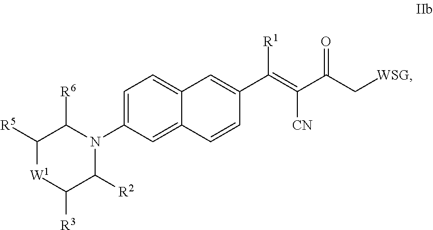
or a pharmaceutically acceptable salt, tautomer or a prodrug thereof.

18. The compound of claim 1, having formula: 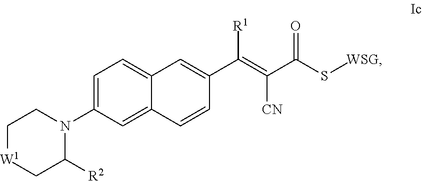
or a pharmaceutically acceptable salt, tautomer or a prodrug thereof.

19. The compound of claim 1, having formula: 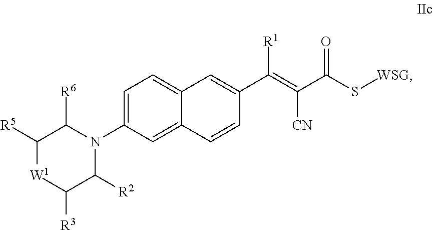
or a pharmaceutically acceptable salt, tautomer or a prodrug thereof.

20. The compound of claim 1, having formula: 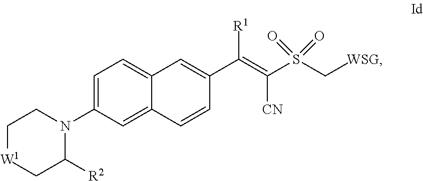
or a pharmaceutically acceptable salt, tautomer or a prodrug thereof.

21. The compound of claim 1, having formula: 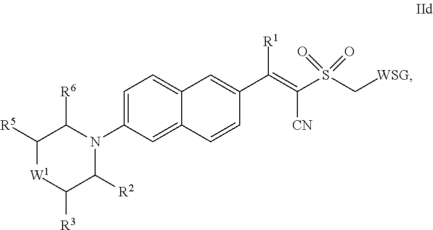
or a pharmaceutically acceptable salt, tautomer or a prodrug thereof.

22. The compound of claim 1, having formula: 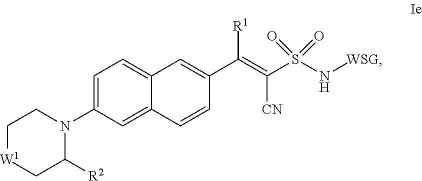
or a pharmaceutically acceptable salt, tautomer or a prodrug thereof.

23. The compound of claim 1, having formula: 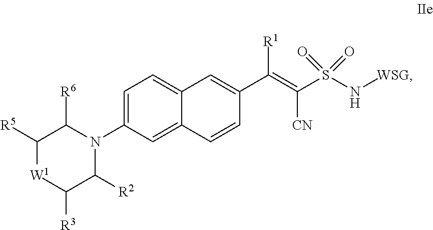
or a pharmaceutically acceptable salt, tautomer or a prodrug thereof.

24. The compound of claim 1, having formula: 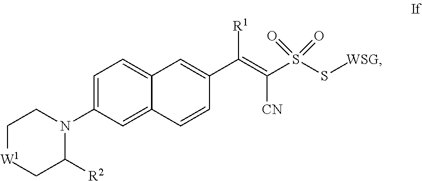
or a pharmaceutically acceptable salt, tautomer or a prodrug thereof.

25. The compound of claim 1, having formula: 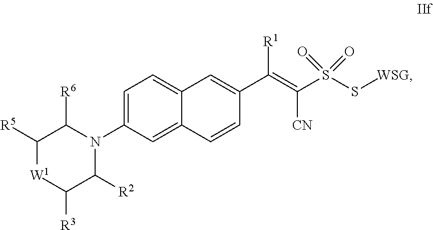
or a pharmaceutically acceptable salt, tautomer or a prodrug thereof.

26. A compound selected from the group consisting of: 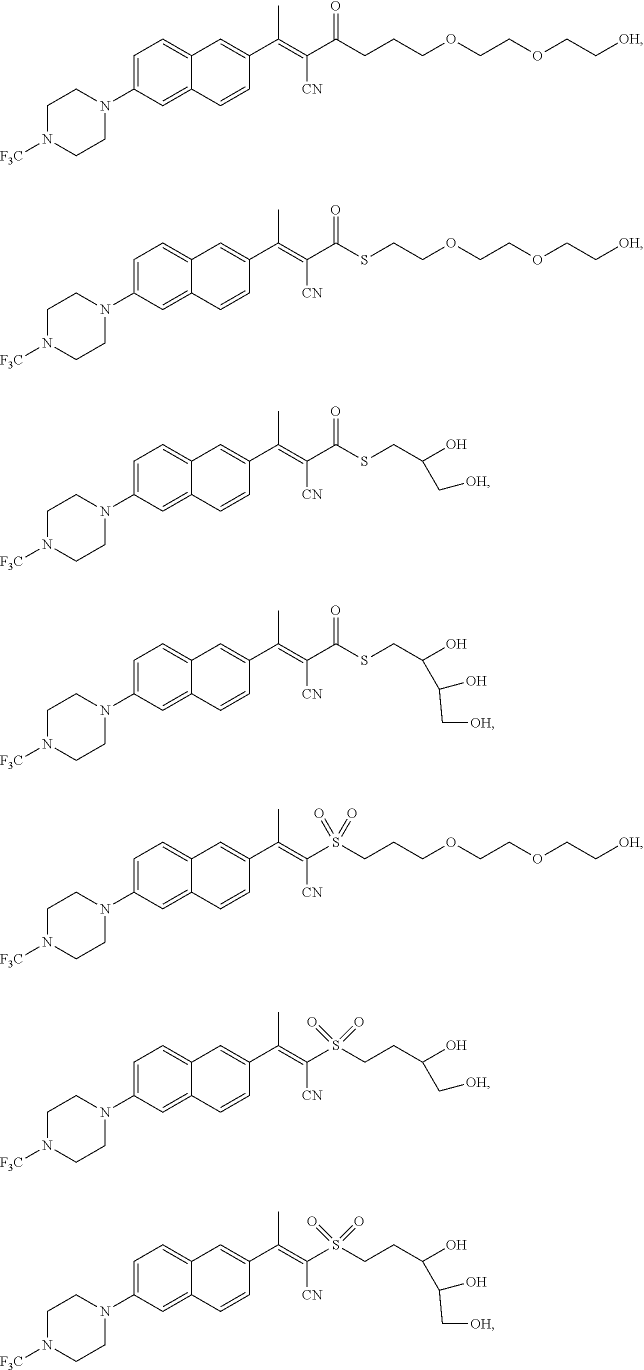 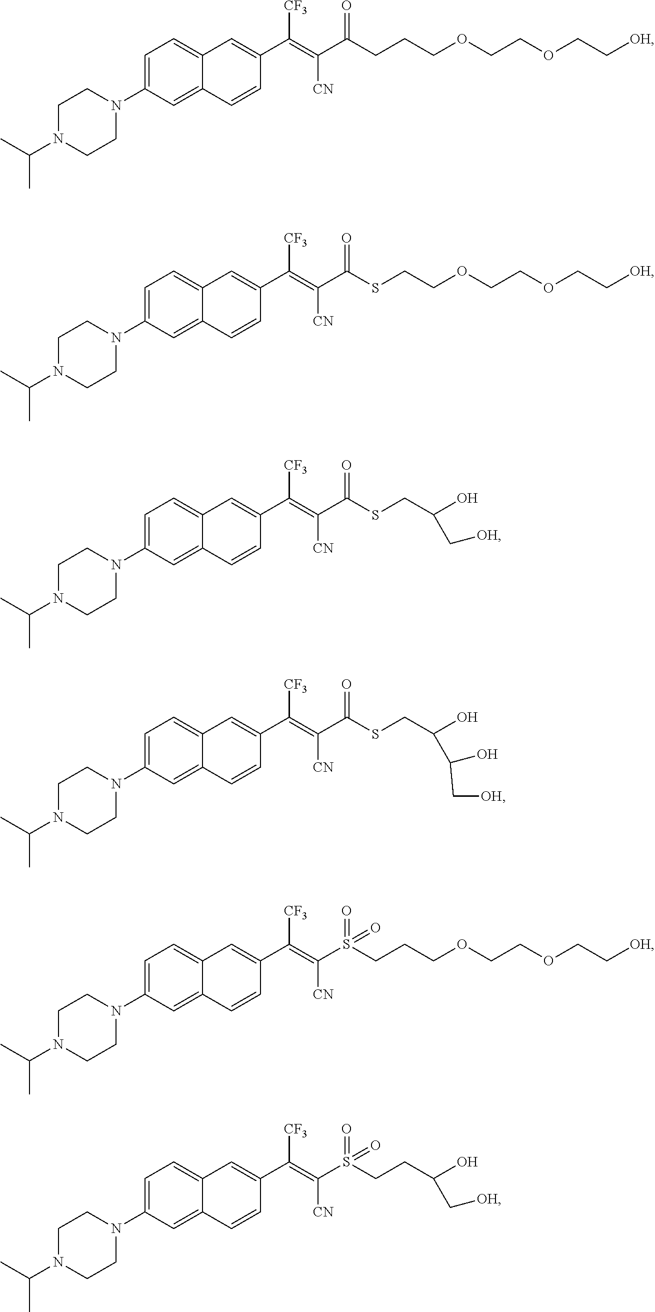 [SMILES: CC(C)N1CCN(c2ccc3cc(/C(=C(\C#N)C(=O)CCCOCCOCCO)C(F)(F)F)ccc3c2)CC1 ; CC(C)N1CCN(c2ccc3cc(/C(=C(\C#N)C(=O)SCCOCCOCCO)C(F)(F)F)ccc3c2)CC1 ; CC(C)N1CCN(c2ccc3cc(/C(=C(\C#N)C(=O)SCC(O)CO)C(F)(F)F)ccc3c2)CC1 ; CC(C)N1CCN(c2ccc3cc(/C(=C(\C#N)C(=O)SCC(O)C(O)CO)C(F)(F)F)ccc3c2)CC1 ; CC(C)N1CCN(c2ccc3cc(/C(=C(\C#N)S(=O)(=O)CCCOCCOCCO)C(F)(F)F)ccc3c2)CC1 ; CC(C)N1CCN(c2ccc3cc(/C(=C(\C#N)S(=O)(=O)CCC(O)CO)C(F)(F)F)ccc3c2)CC1] 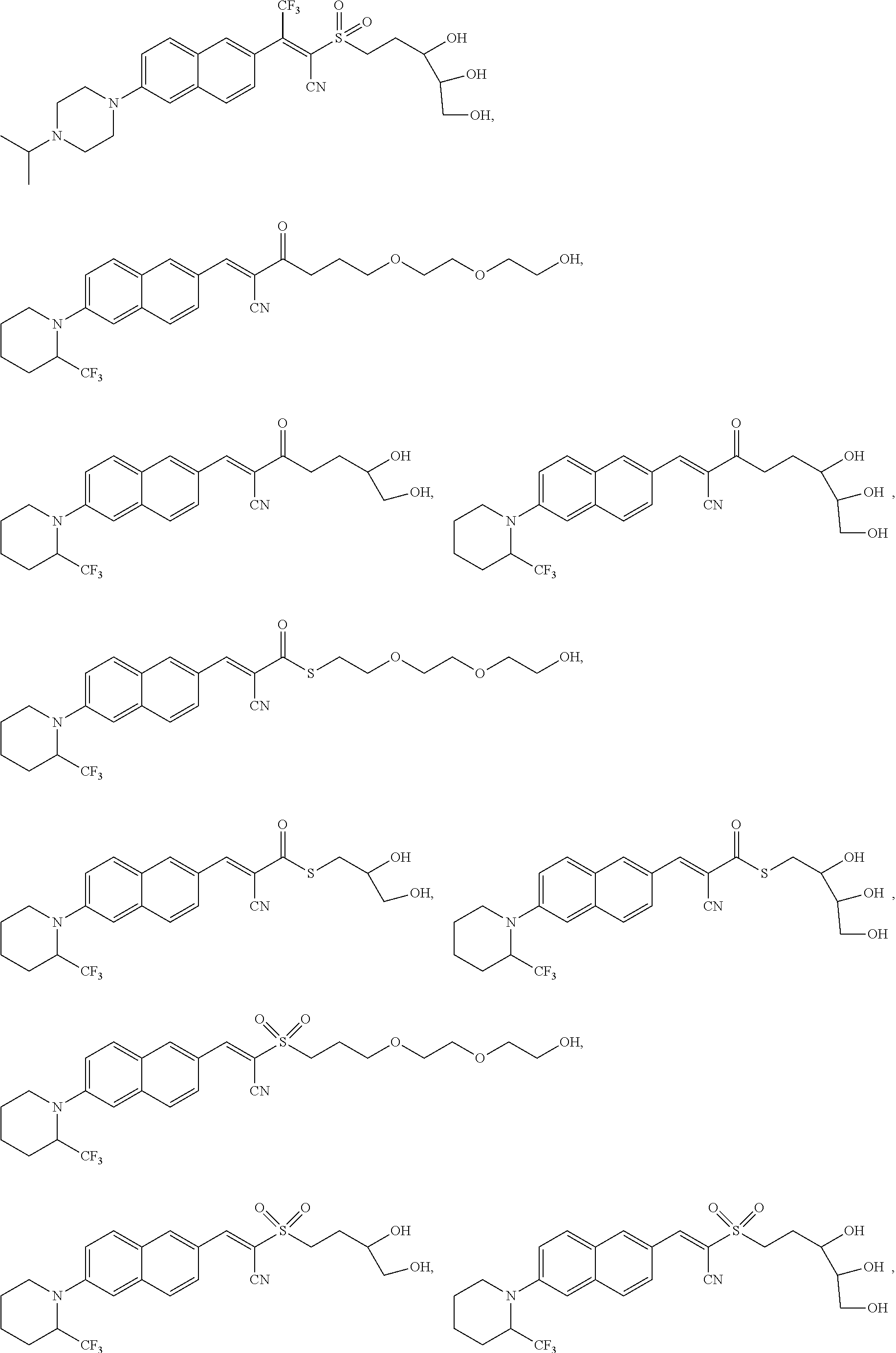 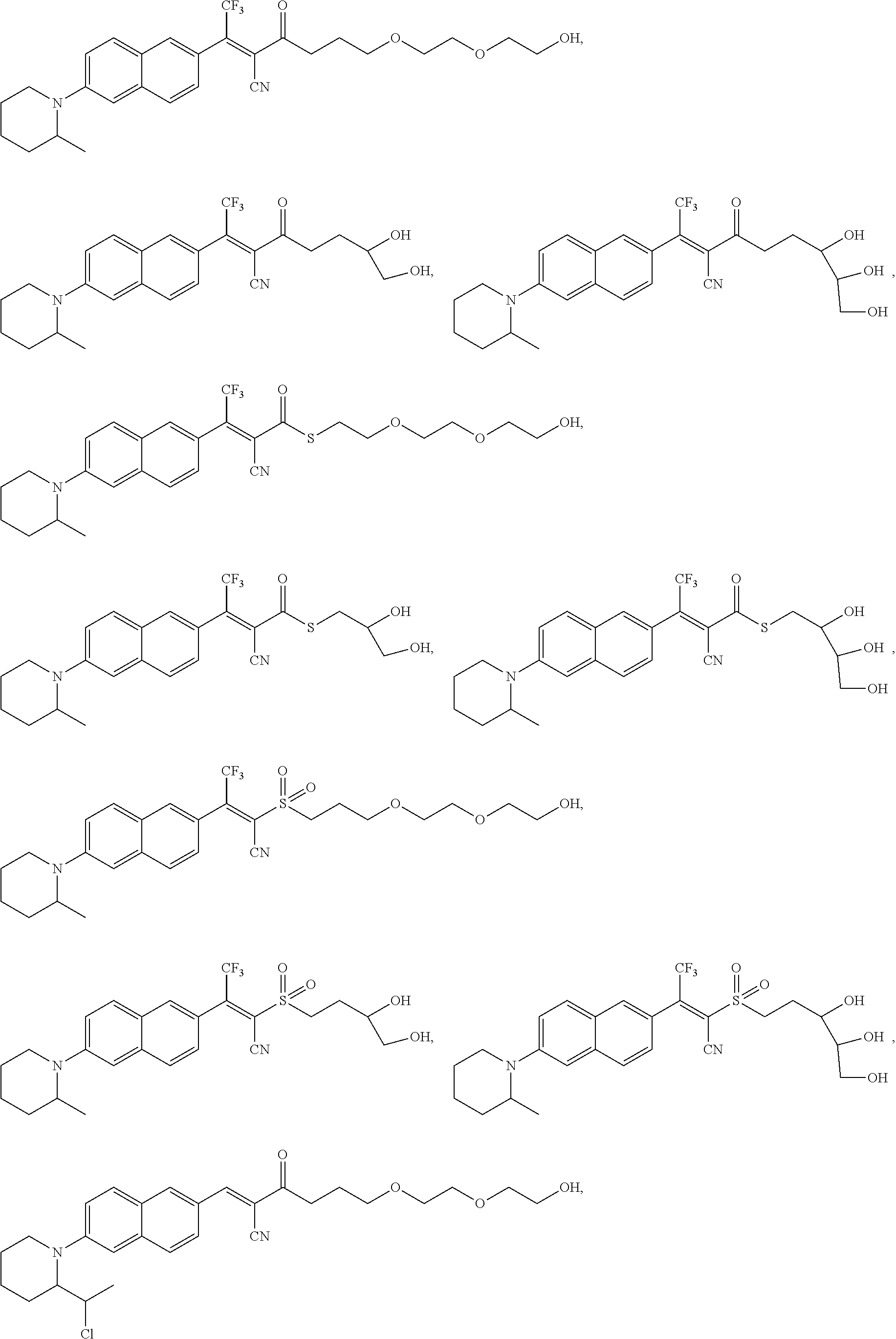 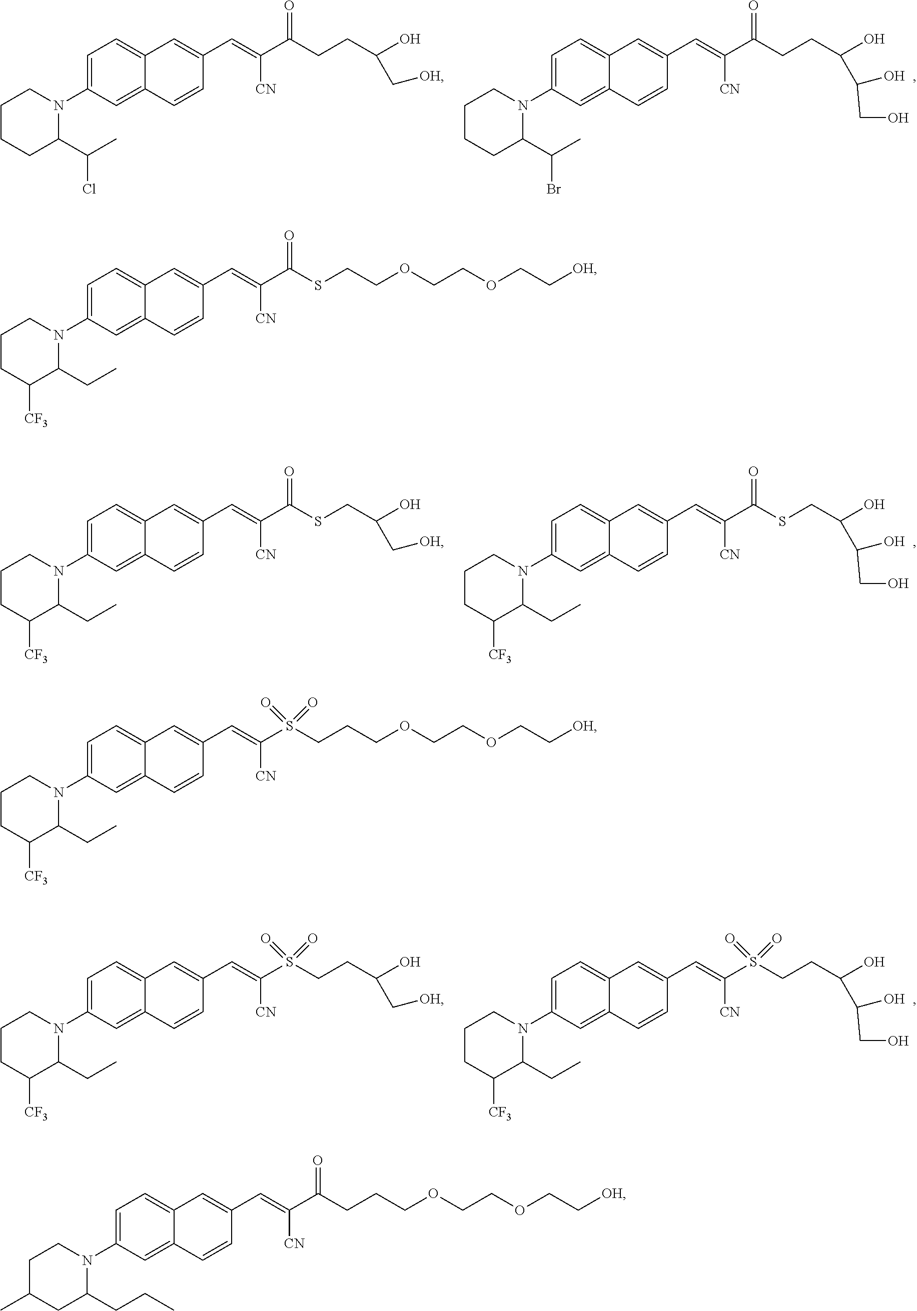 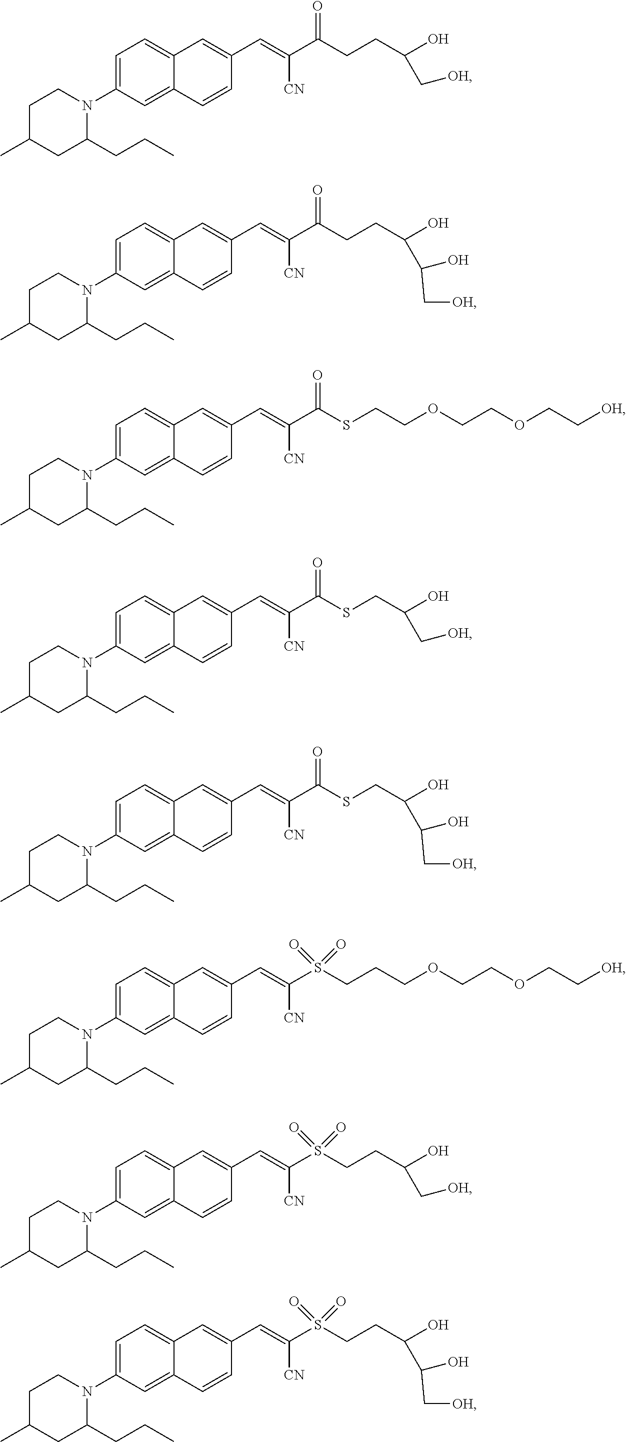 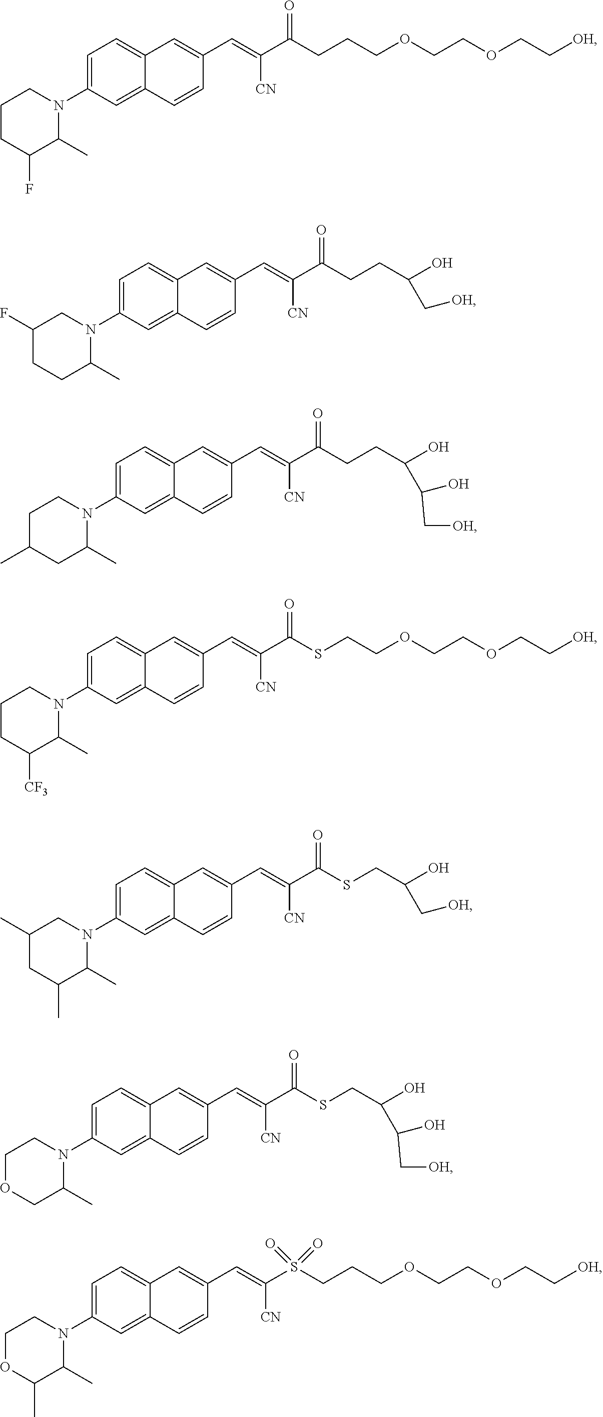 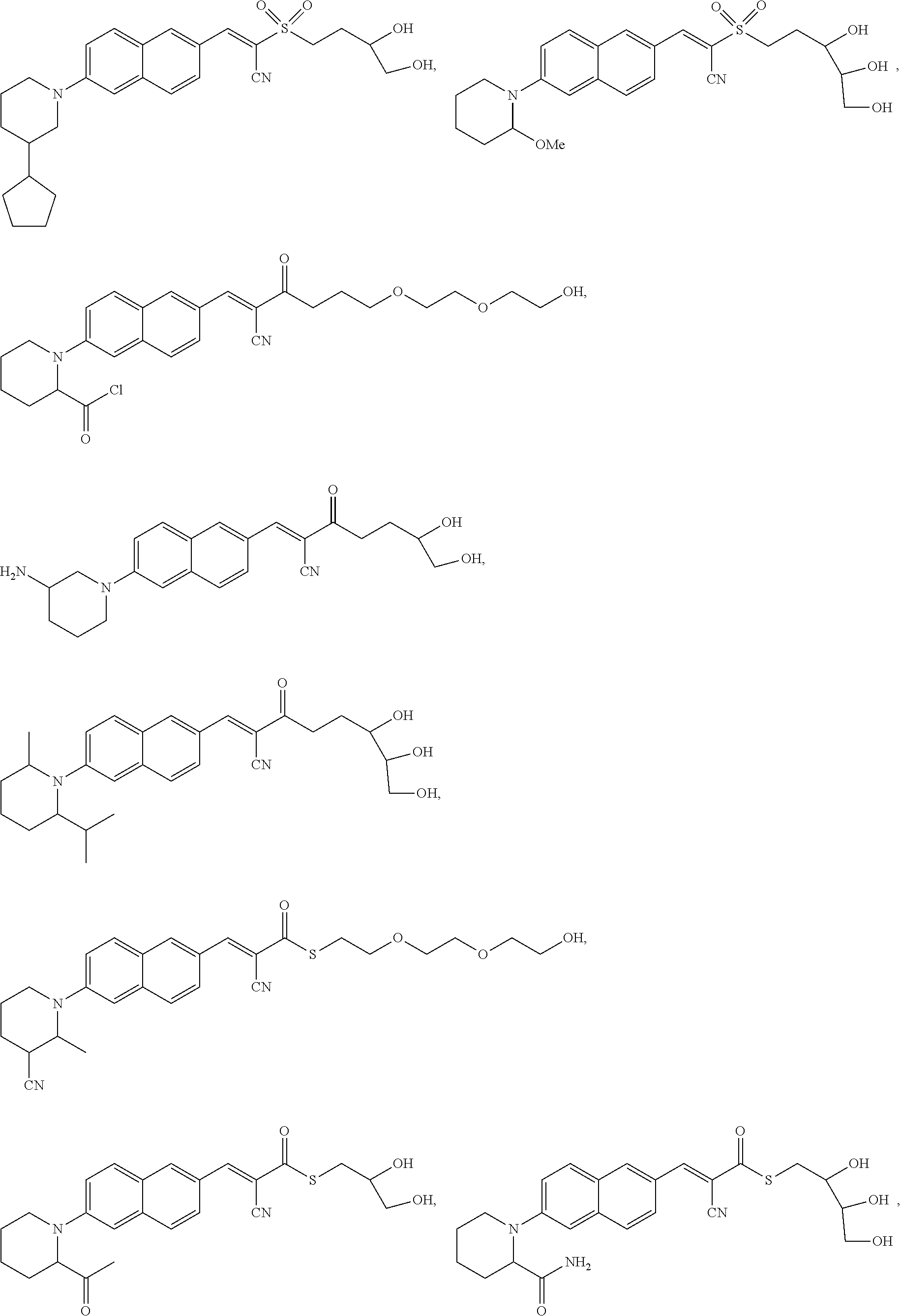 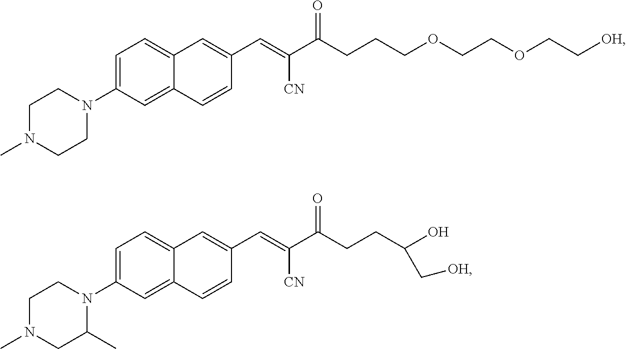 [SMILES: CN1CCN(c2ccc3cc(/C=C(\C#N)C(=O)CCCOCCOCCO)ccc3c2)CC1 ; CC1CN(C)CCN1c1ccc2cc(/C=C(\C#N)C(=O)CCC(O)CO)ccc2c1] 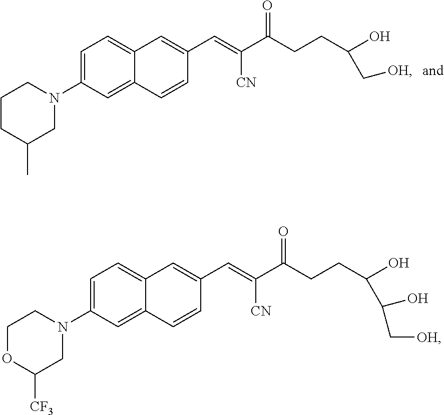 [SMILES: CC1CCCN(c2ccc3cc(/C=C(\C#N)C(=O)CCC(O)CO)ccc3c2)C1 ; N#C/C(=C\c1ccc2cc(N3CCOC(C(F)(F)F)C3)ccc2c1)C(=O)CCC(O)C(O)CO]
or a pharmaceutically acceptable salt, tautomer or prodrug thereof.

27. A pharmaceutical composition comprising a compound of any one of claims 1-26 or a pharmaceutically acceptable salt, tautomer or a prodrug thereof and a pharmaceutically acceptable carrier.

28. A method for determining whether a patient has a neurological disease or disorder, comprising administering to the patient a compound according to any one of claims 1-26, or a pharmaceutically acceptable salt, tautomer or a prodrug thereof, or a pharmaceutical composition according to claim 27.

29. The method of claim 28, wherein the compound is administered intravenously.

30. The method of claim 28, wherein the compound is administered to the eye of the patient.

31. The method of any one of claims 28-30, further comprising detecting the presence or absence of binding of the compound or its parent compound with a detectable target protein.

32. The method of claim 31, wherein the detection comprises activation of a tissue of the patient to be examined by a light thereby producing emission of a detectable signal, and detecting the detectable signal.

33. The method of claim 32, wherein the detectable signal is a fluorescent signal.

34. The method of any one of claims 28-33, wherein the neurological disease or disorder is Alzheimer's disease or traumatic brain injury (TBI).

35. The method of any one of claims 28-33, wherein the neurological disease or disorder is selected from an age-related disease or disorder, a genetic disease or disorder, an injury-related disease or disorder, and a psychiatric disease or disorder.

36. The method of claim 35, wherein the age-related disease or disorder is selected from Parkinson's disease, vascular dementia, and Amyotrophic lateral sclerosis, wherein the genetic disease or disorder is Down syndrome, wherein the injury-related disease or disorder is selected from traumatic brain injury and chronic traumatic encephalopathy, and wherein the psychiatric disease or disorder is selected from schizophrenia and depression.
